(19) United States
(12) Patent Application Publication
(10) Pub. No.: US 2010/0323905 A1
(43) Pub. Date: Dec. 23, 2010

(54) Vhh for the Diagnosis, Prevention and Treatment of Diseases Associated with Protein Aggregates

(75) Inventors: Cornelis Theodorus Verrips, Houten (NL); Silvere Maria Van Der Maarel, Oegstgeest (NL); Peter Verheesen, Gent (BE); David Lutje Hulsik, Nijmegen (NL); Garritjan Boudewijn Van Ommen, Amsterdam (NL)

Correspondence Address: WOLF GREENFIELD & SACKS, P.C., 600 ATLANTIC AVENUE, BOSTON, MA, 02210-2206, US

(73) Assignees: Academisch Ziekenhuis Leiden, Leiden (NL); Universiteit Utrecht Holding B.V., Utrecht (NL)

(21) Appl. No.: 11/992,330

(22) PCT Filed: Sep. 25, 2006
(86) PCT No.: PCT/NL2006/000475

§ 371 Date: Sep. 9, 2010

(30) Foreign Application Priority Data
Sep. 23, 2005 (EP) 055077180.7

(51) Int. Cl. C40B 30/04; C07K 16/18; C07H 21/00; C12N 15/63; C12N 5/22; C12P 21/02; G01N 33/566

(52) U.S. Cl. 506/9; 530/387.1; 536/22.1; 435/320.1; 435/326; 435/69.1; 436/501

(57) ABSTRACT

The present invention provides heavy chain variable domain antibodies (VHH) for preventing and/or dissolving aggregates. VHH of the invention are preferably used in the treatment of human diseases that are associated with the formation of aggregates in the body. The invention further provides, among others, means and methods for selecting and using VHH.

The invention relates to heavy chain variable domain antibodies (VHH) that can be used in the diagnosis, prevention and/or treatment of diseases and disorders that are associated with the undesired formation, build-up and/or presence of proteinaceous aggregates.

The invention in particular relates to heavy chain variable domain antibodies (VHH) that can be used in the diagnosis, prevention and/or treatment of diseases and disorders that are associated with the undesired formation, build-up and/or presence in cells of aggregates of biological materials such as proteins or RNA. Examples of such aggregates and of diseases and disorders associated therewith will become clear from the further description below.

The invention also relates to methods for selecting VHH that can be used in the diagnosis, prevention and/or treatment of such diseases and disorders.

The invention also relates to polypeptides that comprise or essentially consist of such VHH. Some non-limiting examples of such polypeptides will become clear from the further description herein.

The invention also relates to nucleic acids encoding such VHH and polypeptides; to methods for preparing such VHH and polypeptides; to host cells expressing or capable of expressing such VHH or polypeptides; to compositions, and in particular to pharmaceutical compositions, that comprise such VHH, polypeptides, nucleic acids and/or host cells; and to uses of such VHH, polypeptides, nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

There are various diseases and disorders that are associated with, characterized by and/or caused by the undesired formation, build-up or presence of aggregates of biological materials such as proteins or RNA. This may for example, without limitation, be the result of the undesired (over)expression of the genes that encode said biological materials and/or by defects in the biological mechanisms that remove or clear-up such biological materials and/or aggregates. Some non-limiting examples of such diseases and disorders are listed in Table 1.

Thus, it is a general object of the invention to provide compounds and compositions that can be used in the diagnosis, prevention and/or treatment of such diseases and disorders.

It has now been found that heavy chain variable domain antibodies (VHH), which are as further described herein, and polypeptides and compositions comprising the same are preeminently suited for this purpose. In particular, it has been found that such VHH and polypeptides can not only be used to prevent the undesired formation, build-up or further growth of such aggregates, but at least some of them can also be used to remove or reduce the size of such aggregates once they have been formed (removal or reduction in size will be collectively referred to herein as “dissolving” the aggregate). Until this invention it was generally accepted that such an reaction only occurs at the expense of the hydrolysis of ATP [a1].[Haas I G & Wabl M 1983, Nature 306, 387—The aggregates are as further described herein, and may for example be protein aggregates or aggregates of nucleic acids such as RNA. Other examples of such aggregates will be clear to the skilled person based on the disclosure herein.

The aggregates may be present in any organ, part, tissue or cell of a subject in need of treatment, such as a human being. Often, the aggregates will be present in a cell, although the invention in its broadest sense is not limited thereto and also encompasses the use of the VHH and polypeptides described herein to dissolve extracellular aggregates. Again, some non-limiting examples of such aggregates and the organs, tissues or cells in which they may occur will be clear to the skilled person from the further description herein.

The VHH and polypeptides that are used in the invention will depend on the specific aggregate to be dissolved and/or the disease to be treated. In any case, the VHH should be directed against at least one antigenic determinant (e.g. a part of epitope) of the materials (e.g. the protein or nucleic acid) that forms the aggregate, again depending on the specific aggregate to be dissolved. Some particular, but non-limiting, examples of such antigenic determinants will become clear from the further description herein, and for example include specific amino acid repeats or motifs, such as the poly-Ala and poly-Gln repeats referred to below. Thus, in one particular non-limiting embodiment, the VHH and polypeptides of the invention are directed against such antigenic determinants and can be used to dissolve aggregates formed from proteins or other biological materials that contain such antigenic determinants. The invention also provides methods for selecting VHH that are directed against such antigenic determinants and/or that can be used to dissolve specific aggregates and/or to prevent or treat specific diseases. Such methods are as further described herein.

The VHH, polypeptides and compositions of the invention may in particular be used in the prevention, diagnosis and treatment of the diseases and disorders mentioned in Table 1 and/or to dissolve undesired aggregates associated with such diseases and disorders. In particular, the VHH, polypeptides and compositions of the invention may be used in the prevention and treatment of so-called “poly-Gln diseases” (as described herein, with some particular, but non-limiting examples given in Table 1), “poly-Ala diseases” (as described herein, with some particular, but non-limiting examples given in Table 1) and/or “RNA diseases” (as described herein, with some particular, but non-limiting examples given in Table 1), or one of the other aggregation disorders mentioned in Table 1. Again, further examples of such diseases and disorders will be clear to the skilled person based on the further description herein.

The polypeptides used in the present invention may comprise or essentially consist of one or more VHH as described herein. For example, a polypeptide as used in the invention may comprise or essentially consist of one VHH as described herein (a “monovalent” VHH) or may comprise or essentially consist of two or more VHH as described herein (a “multivalent” VHH).

It is also possible to use parts, fragments, analogs, mutants, variants, alleles and/or derivatives (collectively herein “analogs”) of the VHH described herein, and/or to use polypeptides comprising or essentially consisting of one or more of such analogs, as long as these analogs and polypeptides are suitable for the uses envisaged herein, and in particular are capable of dissolving aggregates as described herein. Such analogs and polypeptides will be clear to the skilled person based on the disclosure herein, optionally after some limited experimentation.

According to another non-limiting embodiment, a polypeptide of the invention is a fusion protein that comprises or essentially consists of at least one VHH as described herein and at least one other amino acid sequence (such as a protein or polypeptide), and in particular at least one other amino acid sequence that confers at least one desired property to the VHH and/or to the resulting fusion protein. Such fusion proteins may provide certain advantages compared to the corresponding monovalent VHH. Some non-limiting examples of such amino acid sequences and of such fusion constructs will become clear from the further description herein and/or from the further references cited below.

For example, such an amino acid sequence may form a signal sequence or leader sequence that directs secretion of the VHH from a host cell upon synthesis, as will be clear to the skilled person. Such an amino acid sequence may also form a sequence or signal that allows the VHH to be directed towards and/or to penetrate or enter into specific organs, tissues, cells, or parts or compartments of cells, and/or that allows the VHH to penetrate or cross a biological barrier such as a cell membrane, a cell layer such as a layer of epithelial cells, a tumor including solid tumors, or the blood-brain-barrier. Examples of such amino acid sequences will be clear to the skilled person. Some non-limiting examples are the small peptide vectors (“Pep-trans vectors”) described in WO 03/026700 and in Temsamani et al., Expert Opin. Biol. Ther., 1, 773 (2001); Temsamani and Vidal, Drug Discov. Today, 9, 1012 (004) and Rousselle, J. Pharmacol. Exp. Ther., 296, 124-131 (2001), and the membrane translocator sequence described by Zhao et al., Apoptosis, 8, 631-637 (2003). C-terminal and N-terminal amino acid sequences for intracellular targeting of antibody fragments are for example described by Cardinale et al., Methods, 34, 171 (2004). Other suitable techniques for intracellular targeting involve the expression and/or use of so-called “intrabodies” comprising a VHH as described herein, as for example as described in WO 94/02610, WO 95/22618, U.S. Pat. No. 6,004,940, WO 03/014960, WO 99/07414; WO 05/01690; EP 1 512 696; and in Cattaneo, A. & Biocca, S. (1997) Intracellular Antibodies: Development and Applications. Landes and Springer-Verlag; and in Kontermann, Methods 34, (2004), 163-170, and the further references described therein.

According to a specific, but non-limiting embodiment, a polypeptide of the invention comprises or essentially consists of at least one VHH as described herein and at least one other VHH (i.e. directed against another epitope, antigen, target, protein or polypeptide). Such proteins or polypeptides are also referred to herein as “multispecific” proteins or polypeptides or as “multispecific constructs”, and these may provide certain advantages compared to the corresponding monovalent VHH. Again, some non-limiting examples of such multispecific constructs will become clear from the further description herein and/or from the references cited herein.

For example, such a further VHH may direct the VHH or polypeptide towards specific organs, tissues, cells, or parts or compartments of cells and/or may allows the VHH or polypeptide to penetrate or to enter into the same, and/or may allow the VHH or polypeptide to penetrate or cross a biological barrier such as a cell membrane, a cell layer such as a layer of epithelial cells, a tumor including solid tumors, or the blood-brain-barrier. Examples of such VHH include VHH that are directed towards specific cell-surface proteins, markers or epitopes of the desired organ, tissue or cell (for example cell-surface markers associated with tumor cells), and the single-domain brain targeting antibody fragments described in WO 02/057445.

It is also possible to combine two or more of the above embodiments, for example to provide a trivalent bispecific construct comprising two VHH as described herein and one other VHH, and optionally one or more other amino acid sequences. Further non-limiting examples of such constructs, as well as some constructs that are particularly preferred within the context of the present invention, will become clear from the further description herein.

In the above multivalent and/or multispecific constructs, the one or more VHH and/or other amino acid sequences may be directly linked or linked via one or more linker sequences. Suitable examples of such linkers will be clear to the skilled person (see for example the art cited below), and for example also include all linkers used in the art to link antibody fragments (for example to form ScFv fragments).

For a general description of multivalent and multispecific constructs and their design and preparation, reference is also made to Conrath et al., J. Biol. Chem., Vol. 276, 10. 7846-7350, 2001, as well as to for example WO 96/34103 and WO 99/23221.

It is also possible to use derivatives of the VHH and polypeptides described herein, in which the VHH or polypeptide is linked (e.g. covalently attached) to one or more functional groups. Examples of such functional groups and methods for linking the same to the VHH and polypeptides described herein will be clear to the skilled person, and for example include all functional groups known in the art to modify antibodies or antibody fragments. For example, for diagnostic purposes, the VHH or polypeptide may be linked to a detectable moiety or to a(nother) signal-generating groups or moieties, depending on the intended use of the labelled VHH. Suitable labels and techniques for attaching, using and detecting such groups will be clear to the skilled person, and for example include, but are not limited to, fluorescent labels (such as fluorescein, isothiocyanate, rhodamine, phycoerythrin, phycocyanin, allophycocyanin, o-phthaldehyde, and fluorescamine and fluorescent metals such as 152Eu or others metals from the lanthanide series), phosphorescent labels, chemiluminescent labels or bioluminescent labels (such as luminal, isoluminol, theromatic acridinium ester, imidazole, acridinium salts, oxalate ester, dioxetane or GFP and its analogs), radio-isotopes (such as 3H, 125I, 32P, 35S, 14C, 51Cr, 36Cl, 57Co, 58Co, 59Fe, and 75Se), metals, metals chelates or metallic cations (for example metallic cations such as 99 mTc, 123I, 111In, 131I, 97Ru, 67Cu, 67Ga, and 68Ga or other metals or metallic cations that are particularly suited for use in in vivo, in vitro or in situ diagnosis and imaging, such as (157Gd, 55Mn, 162Dy, 52Cr, and 56Fe), as well as chromophores and enzymes (such as malate dehydrogenase, staphylococcal nuclease, delta-V-steroid isomerase, yeast alcohol dehydrogenase, alpha-glycerophosphate dehydrogenase, triose phosphate isomerase, biotinavidin peroxidase, horseradish peroxidase, alkaline phosphatase, asparaginase, glucose oxidase, β-galactosidase, ribonuclease, urease, catalase, glucose-VI-phosphate dehydrogenase, glucoamylase and acetylcholine esterase). Other suitable labels will be clear to the skilled person, and for example include moieties that can be detected using NMR or ESR spectroscopy. Such labelled VHH and polypeptides of the invention may for example be used for in vitro, in vivo or in situ assays (including immunoassays known per se such as ELISA, RIA, EIA and other “sandwich assays”, etc.) as well as in vivo diagnostic and imaging purposes, depending on the choice of the specific label.

In another aspect, the invention relates to host or host cell that expresses or that is capable of expressing a VHH as described herein and/or a polypeptide as described herein; and/or that contains or expresses a nucleic acid encoding the same. In principle, any suitable host cell or expression system can be used, and examples thereof will be clear to the skilled person, for example from the further references cited herein.

The invention further relates to a product or composition containing or comprising at least one VHH as described herein, at least one polypeptide as described herein, and/or at least one nucleic acid encoding the same, and optionally one or more further components of such compositions known per se, i.e. depending on the intended use of the composition. Such a product or composition may for example be a pharmaceutical composition (as described herein), a veterinary composition or a product or composition for diagnostic use (as also described herein). Some preferred but non-limiting examples of such products or compositions will be clear from the references cited below and/or from the further description herein.

The invention further relates to methods for preparing or generating the VHH, polypeptides, nucleic acids, host cells, products and compositions described herein. Some preferred but non-limiting examples of such methods will become clear from the further description herein.

The invention further relates to applications and uses of the VHH, polypeptides, nucleic acids, host cells, products and compositions described herein, as well as to methods for the prevention and/or treatment for diseases and disorders associated with an undesired formation, build-up or presence of aggregates. Some preferred but non-limiting applications and uses will become clear from the further description herein.

Other aspects, embodiments, advantages and applications of the invention will also become clear from the further description herein below.

The VHH of the invention may be generated in any manner known per se, which will be clear to the skilled person. Generally, this will involve at least one step of selecting VHH that are directed against at least one epitope or antigenic determinant on the protein or material that forms the aggregate, and preferably also at least one further step of selecting (i.e. from the VHH thus selected) VHH that are capable of dissolving the desired aggregate(s). The first selection step can be performed in any manner known per se for selecting VHH or antibodies against a desired antigen, such as the techniques reviewed by Hoogenboom, Nature Biotechnology, 23, 9, 1105-1116 (2005), the so-called SLAM technology (as for example described in the European patent application 0 542 810), the use of transgenic mice expressing human immunoglobulins or the well-known hybridoma techniques (see for example Larrick et al, Biotechnology, Vol. 7, 1989, p. 934). The second step can generally be performed using any suitable in vitro, cell-based or in vivo assay (depending on the specific aggregate) and suitable assays will be clear to the skilled person based on the disclosure herein.

Typically in selections starting with an immune library the number of phages is reduced from 10^{e}7 to 10^{e}4; whereas in selections starting with a non-immunized library the number of phages is reduced from about 10^{e}9 to 10^{e}4. The selection is based on binding of the phage to the antigen of choice. In the subsequent steps, consisting of DNA finger printing of the selected VHH genes, production in E. coli and a lower eukaryote to evaluate the folding properties of the selected VHH in vivo, the number of positive phages is generally reduced from 10e4 to 10e2. The screening on in vivo folding properties of the selected VHHs selects for their functionality in- and outside cells. It has been found that there is a strong correlation between correct folding in vivo and the [re]folding of VHHs in vitro. After this screening typically 20-50 VHHs remain suitable candidates and from these candidate VHHs the nucleotide sequences are determined, which also provide the amino acid sequences. Finally the thus screened positive VHHs are tested under the actual conditions of the disease to be combated. In this invention we describe the expression of a protein causing intracellular aggregation and the simultaneous expressing of the VHHs of which the screening was positive and of which the amino acid sequences are different. The final screening process normally reduces the number of candidate VHHs to less than 20% of the VHHs for which the amino acid sequence have been determined. Although prevention of aggregates is preferred to combat aggregate associated diseases, even more preferred is to screen VHHs on their property to dissolve existing aggregates. This screening can be performed by synthesis of the aggregate forming protein prior to the expression of the VHHs.

Once generated, the VHH (or analogs thereof) and the polypeptides comprising the same may be prepared in any suitable manner known per se, as further described below.

Antibodies and antibody-derivates are among the most preferable tools to study gene products and their functions in in vitro systems and in natural contexts[b1-3] [Bradbury A et al 2003a, Trends in Biotechnol 21, 275-281; Bradbury et al 2003b, Trends in Biotech 21, 312-317; Hust M & Dubel S 2004, Trends in Biotech. 22, 8-14].[1-3]. Yet, traditional antibody generation methods suffer from several limitations including the troublesome production of antibodies against proteins with high interspecies homology, against membrane proteins and the difficult generation of large sets of monoclonal antibody sources in a timely and cost-effective fashion.

Some of these shortcomings can be overcome by using antibody display libraries, where antibodies typically consisting of the variable domains of the heavy and light chains are expressed on the surface of carriers such as by fusion to an endogenous phage coat protein [McCafferty J. et al 1990, Nature 348, 552-554; Smith G P Science 228, 1315-1317]. Both immune [b6][Clackson T et al. 1991, Nature 352, 624-628 and nonimmune [b4] [McCafferty et al 1990, Nature 348, 552-554] repertoires have been successfully used to construct antibody display libraries. However, technical limitations related to the random combination of heavy and light chain V-genes as opposed to the selective combination of in vivo matured IgG molecules, render the majority of display-derived antibodies non-functional or unspecific as most naturally occurring V-gene combinations will be lost [b7] [Marks J D 1992, Biotechnology 10, 779-783].

The combination of antibody display techniques with fragments derived from the variable domains of heavy-chain antibodies (VHH) [b8, 9][Arbabi Ghahroudi M et al. 1997, FEBS Lett. 414, 512-516; v.d. Linden R et al 2000, J. Immunol. Methods 240, 185-195] is an emerging source of naturally occurring antibody binding domains that can be utilized beyond such technical restrictions. Camelidae express in addition to their conventional immune repertoire, an equally common repertoire of heavy-chain antibodies that consist solely of two identical heavy chain molecules. Consequently, the antigen-binding domain of each antibody is encoded by a single gene, rather than the combination of variable domain heavy chain (VH) and light chain (VL) genes. To compensate for the lack of light chains, heavy-chain antibody variable domains have undergone several adaptations, the most prevalent being the expansion of complementarity determining region (CDR) 3 and often the use of so called frame work residues, thereby creating an interface between antigen and VHH comparable to that of conventional antibodies [b10] Desmyter A et al. 1996, Nature Struct Biol 3, 803-811]. Display techniques of nonimmune heavy-chain antibody sources combined with a highly efficient selection and screening strategy allow fast and cost-effective establishment of large panels of high affinity VHH, in particular for disease-related proteins. In the present invention, VHH and VHH-like molecules are referred to as VHH.

VHH-selection from libraries has started with the well-known phage display technology. However, presently many different display technologies are available. Display technologies all couple the VHH to a carrier comprising nucleic acid that encodes at least the antigen binding specificity of the VHH. Display libraries thus all contain VHH associated with carriers that contain said nucleic acid. As a result of this coupling the selection of the VHH results in the selection of the encoding nucleic acid. This nucleic acid can be sequenced or be used to amplify the selected VHH with or without the carrier.

In one aspect the present invention provides a method to isolate specific VHH, such as from non-immune VHH phage display libraries. The VHH are selected in a sequential selection protocol wherein VHH selected in a first round are used as starting material in a second round. The selected subset may be used directly in the subsequent round of selection. Typically, however, the subset is first amplified before initiation of the subsequent selection round. Thus in aspect the invention provides a method for selecting an antigen specific VHH carrier from a display library comprising a plurality of VHH carriers said method comprising at least two successive rounds of antigen binding directed selection of VHH carriers, wherein in one round of selection VHH carriers are selected from said library through contacting VHH carriers with directionally immobilized antigen and wherein in another round of selection, antigen specific VHH carriers are selected by contacting VHH carriers with passively immobilized antigen. Selection using said one round of directionally immobilized antigen and said one round of passively immobilized antigen is especially effective in generating selected VHH with a high affinity for antigen in its natural conformation, in particular in complex with proteins that interact with the antigen. In a preferred embodiment the invention provides said method wherein in one round of selection a subset of VHH carriers is selected from said library through contacting said library with directionally immobilized antigen and wherein in a subsequent round of selection said antigen specific VHH carrier is selected from said subset by contacting said subset or a part thereof with passively immobilized antigen. This order of selection steps results in more and more specific VHH with a high affinity for antigen in its natural conformation, in particular in complex with proteins that interact with the antigen.

The at least two selection rounds are preferably followed by at least one screening round wherein two or more VHH or VHH carriers individually are tested for the property to at least in part prevent aggregation of protein comprising said antigen and/or for the property to at least in part dissolve aggregates comprising protein comprising said antigen in vivo and/or in vitro.

In a selection round of the invention, VHH and/or VHH carriers are selected from a larger collection on the basis of affinity for antigen under the conditions used. Preferably, these conditions are as close as possible to the conditions characteristic for the disease. In a screenings round, functional properties of VHH and/or VHH carriers comprising affinity for said antigen, are scrutinized. The results of the screenings round are typically used to select one or more VHH and/or VHH carriers from the collection entered in the screenings round. Thus a selection round typically selects VHH or VHH-carriers having affinity for the antigen from a larger collection containing VHH and VHH that do not bind to the antigen under the conditions used. A screenings round typically tests VHH or VHH carriers for the property of a protein or aggregate of protein comprising the antigen that the VHH has affinity for. In the present invention the tests entail the at least partial prevention of aggregation of proteins comprising said antigen and/or the property to at least partly dissolve an existing aggregate comprising protein comprising said antigen. Selection and screening rounds are preferably performed sequentially. However, a selection and screening can also be combined. For instance, a screenings round may comprise VHH or VHH carriers that do not bind, or from which it is not known that they bind to said antigen under the conditions used. Vise versa, a selection round may include a test for functionality of VHH and/or VHH carriers.

Directional immobilization of antigen can be achieved in a variety of ways. Directionality is typically achieved through affinity interaction of the antigen with a specific binding member. Said member can be a specific ligand or receptor for the antigen but is typically an antibody or VHH. This type of binding has the advantage that the antigen can be immobilized in its natural conformation. Immobilization of the antigen can be together with proteins that the antigen normally associates and/or forms a complex with however, this is not a requirement. Passive immobilization is typically performed by immobilizing the antigen through non-specific interaction. The term immobilization is used herein to refer to the association of the antigen with a solid phase. Association with a solid phase allows separation of the antigen (and associated VHH or VHH carriers) from the surrounding medium. Non-limiting solid phases are plastic, glass and metal. Metal is typically used in the form of beads. Beads that can be magnetized are often used. Immobilization of the antigen is typically done prior to exposing the antigen to the VHH carriers. However, this is not necessary. For instance, directional immobilization is particularly suited to immobilize the antigen after association with the VHH carriers. In this embodiment, antigen-VHH carrier complex can be separated from the surrounding medium (containing unbound VHH carrier) by contacting said medium with said specific binding member. If the specific binding member is associated with a solid phase, the antigen-VHH complex is immobilized upon binding of the specific binding member to the antigen. Separation of surrounding medium can subsequently proceed as usual. Thus in a preferred embodiment said directionally immobilized antigen is immobilized on a solid surface by means of a specific binding member that is specific for an epitope on said antigen. Such an epitope can also be an “artificially introduced” epitope like an C- or N terminal myc, his, VSV, V5 or an C terminal biotin tag by in vivo biotinylation.

Selection processes entail a selection criterion with which some members of a collection are separated/isolated from other members of the collection. Selection methods of the present invention are based on antigen binding directed selection. This means that members of a collection of VHH carriers (the starting library or subsets resulting from one or more selection rounds) are separated from the other members based on their affinity for the antigen under the conditions used in the selection round. A selection round typically comprises at least one step wherein a collection of VHH carriers is contacted with antigen under chosen binding conditions, and at least one step of separating antigen bound from unbound VHH carriers under chosen washing conditions. By choosing appropriate binding and washing conditions one can select VHH carriers with particular antigen binding characteristics

An antigen is typically a protein. The protein is preferably a protein as occurring in nature, or a part thereof. Antigen can be a protein or a part thereof that is associated with as disease. The antigen can, for instance, be a mutant form of a protein as occurring in nature. Apart from proteins occurring in nature, the antigen can also be a processed form thereof, or be artificial/synthetic. In a preferred embodiment said antigen is an antigen of a protein encoded by a a mammalian, preferably a primate gene. Mammals and particularly priomates are closely related to humans and posses many proteins that function in human cells and that share epitopes with human proteins. Thus, a person skilled in the art can select a VHH or VHH carrier capable of specifically binding to a human protein by selecting with antigen derived from a mammal and preferably a primate. Antigen can of course also be a chimeric of a non-human mammal protein and the corresponding human protein. In a preferred embodiment, said mammalian gene is a primate gene, more preferably a human gene.

A part of a protein, as used herein, typically comprises at least 10 and preferably at least 20 consecutive amino acids. Said protein is preferably a protein encoded by a gene that is associated with a disease in humans. In a preferred embodiment said disease is associated with accumulation of aggregates comprising at least said protein or a mutant thereof. It is preferred that the antigen comprising said protein or parts or derivatives thereof are normally present in said aggregates associated with human disease. Protein that is incorporated into said aggregates can be the protein as encoded by the gene in the genome or be a part of at least 10 and preferably at least 20 consecutive amino acids of said protein. The part, is typically generated by through the action of enzymes. For instance, in the case of β-ameloid, peptidic fragments are generated or over-produced by a deregulated and/or mutated enzyme which results in the incorporation of said peptidic fragments in aggregates. As mentioned above, a method of the invention is particularly suited for selecting VHH carriers that are specific for antigens in their natural conformation. A conformation as occurring in nature includes all shapes, size and alterations that can be found on antigens in nature. Often the antigen is a protein encoded by a gene in the genome of a human or other mammals. However, antigen can also be a processed form of said protein, including but not limited to said antigen comprising one or more posttranslational modifications and/or one or more proteolytic fragments comprising at least 10 and preferably at least 20 consecutive amino acids of said protein. A conformation as occurring in nature also includes mutants occurring in humans/mammals comprising one or more alterations in the amino acid sequence when compared to the protein in healthy individuals. A conformation of the antigen as occurring in nature is preferably the conformation of the antigen that is associated with the formation of aggregates. It is typically the predominant folding form of the antigen in the aggregation area(s). However, folding forms that are intermediates between the unfolded and a completely folded form are also natural conformations according to the present invention.

It has been found that the present a method of the present invention is further suited for selecting VHH carriers specific for antigens that are associated with the inappropriate formation of proteinaceous aggregates in humans. The invention therefore preferably provides a method of the invention wherein said protein is a protein encoded by a gene of table 1. In a preferred embodiment said gene is PABPN1 or IT15.

Aggregation of proteins in proteinaceous aggregates can occur with normal a protein, i.e. which has an amino acid sequence that is identical to an amino acid sequence found in healthy individuals. However, aggregation is typically associated with mutant forms of a protein when compared to the protein in healthy individuals, or with proteins that are processed by mutated enzymes or enzymes of which the expression is deregulated due to a mutation in a regulatory sequence of said enzyme or due to age-related changes in expression. In these cases, a method of the invention is preferably performed using antigen derived from a normal protein (i.e. having an amino acid sequence that is not the mutant form that is associated with aggregation). Thus in a preferred embodiment the invention provides a method wherein said disease is associated with aggregates comprising a mutant of said protein. VHH carriers can be selected for the function of being capable of at least partially inhibiting the formation of aggregates, even when aggregation is associated with a mutant form of said protein. It has been found that at least some of the selected VHH carriers and VHH derived therefrom can at least partially dissolve already formed aggregates. Thus in a particularly preferred embodiment a method of the invention further comprises at least one round of screening wherein said screening comprises contacting selected VHH carriers or VHH derived therefrom with antigen of a protein encoded by a normal mammalian gene under in vivo or in vitro conditions that otherwise stimulate the formation of aggregates and/or in the presence of formed aggregates. In a preferred embodiment said conditions comprise a cell producing antigen in the form that aggregates.

In view of the above the invention in one aspect, provides a method for selecting an antigen specific VHH carrier from a display library comprising a plurality of VHH carriers said method comprising selecting said antigen specific VHH carrier from said display library by means of at least two successive rounds of antigen binding directed selection of VHH carriers, wherein said antigen is an antigen of a protein encoded by a mammalian gene; said gene being associated with accumulation of aggregates in humans; and wherein said antigen is an antigen of a protein encoded by the normal mammalian gene. Preferably said gene is a human gene. In a preferred embodiment of this aspect of the invention said antigen specific VHH carrier is selected by a method described herein above, i.e. a method for selecting an antigen specific VHH carrier from a display library comprising a plurality of VHH carriers said method comprising at least two successive rounds of antigen binding directed selection of VHH carriers, wherein in one round of selection VHH carriers are selected from said library through contacting VHH carriers with directionally immobilized antigen and wherein in another round of selection, antigen specific VHH carriers are selected by contacting VHH carriers with passively immobilized antigen; and preferred embodiments thereof. One particularly preferred embodiment entails that, as mentioned above, the at least two selection rounds are preferably followed by at least one screening round wherein two or more VHH or VHH carriers are tested for the property to at least in part prevent aggregation of proteins comprising said antigen and/or for the property to at least in part dissolve aggregates comprising proteins comprising said antigen.

Diseases that are associated with the formation of proteinaceous aggregates in individuals produce aggregates that consist of more components than just a gene product of a gene that is associated with the formation of said aggregates in said disease. Typically said aggregates comprise other proteins and/or RNA. The product of said gene that is actually incorporated can also be RNA. In these cases, the antigen comprises a protein and/or part thereof, that is also incorporated into said aggregate. In these cases said protein and/or part thereof, is preferably encoded by another gene of table 1.

A screening round of the invention is preferably performed by cloning at least two nucleic acids encoding VHH from at least two selected VHH-carriers each into a VHH-expression vector. For said preferred screening round said expression vectors are introduced into a model cell line that produces aggregates comprising said antigen, said preferred screening round further comprising determining whether expression of said cloned VHH at least in part prevents the formation of said aggregates and/or determining whether said cloned VHH at least in part dissolves said aggregates. In another preferred embodiment, said expression vectors are used to produce the corresponding VHH and it is determined whether one or more of said produced VHH at least in part prevent the formation of said aggregates and/or determined whether one or more of said produced VHH at least in part dissolve said aggregates in an in vitro system for the formation of said aggregates. Preferably, said in vitro system comprises already formed aggregates, preferably naturally formed aggregates. Non-limiting examples of such model cell lines and in vitro systems are well known in the art Said in vitro system are particularly preferred for antigens of proteins that are associated with diseases with extra-cellular aggregates.

Antigens may have immunodominant epitopes. Immunodominant epitopes are epitopes of which the used library has a high number of VHH carriers that are specific for said epitope. Alternatively, the VHH carrier specific for said epitope is easily selected and or amplified between selections. In general immunodominant epitopes are herein defined as epitopes of an antigen that yield a particularly high amount of specific VHH carriers in a method of the invention. It can be desired to select antigen specific VHH carriers that are specific for immunodominant epitopes. However such sites are often also involved in binding other biomolecules and is not available in vivo. In such cases one has to determine first these other molecules, e.g. by immunoprecipitation followed by determination of (part of) the amino acid sequence of the precipitated and subsequently separated proteins. If other antigen specific VHH carriers are desired, the occurrence of immunodominant epitopes on an antigen may reduce the number of antigen specific VHH carriers. In these cases one can mask such immunodominant epitope. One way is to provide antigen in which the immunodominant epitope is mutated such that it no longer functions as an immunodomant epitope. In a preferred embodiment, however, said immunodominant epitope on said antigen is masked prior to contacting VHH with said antigen in a selection round. Masking can of course also be done with epitopes that axe undesired for other reasons then immunodominance. In a preferred embodiment at least one amino acid repeat is at least partially masked. Preferably, said amino acid repeat comprises a poly-Ala stretch or a poly-Gln stretch. Such a stretch comprises at least 4 consecutive Ala or Gln amino acids. In a particularly preferred embodiment said amino acid repeat is masked by a VHH, wherein binding of said VHH is dependent on said repeat but leaves at least one residue of the (extended) repeat free as well sequentially or structurally adjacent non-repeat amino acids involved or even essential for aggregate formation. In such a way aggregation preventing-VHHs or aggregate dissolving VHHs can be selected that are recognize with low affinity the amino acid(s) of the expansion and with high affinity for sequentially or structurally adjacent amino acid.

In yet another preferred embodiment at least one epitope on said antigen is masked with a VHH specific for said antigen, wherein said VHH does not affect aggregation and/or does not dissolve formed aggregate. This embodiment is useful in selection rounds to select VHH (carriers) that bind to different epitopes on said antigen. Masking VHH can, for instance, be derived from previous selection and screening rounds. Use of masked antigen increases the proportion of candidate inhibitor or dissolver VHH in the set of selected VHH specific for said antigen in a method of the invention.

Selected VHH carriers may be used directly. However, typically the nucleic acid encoding at least the antigen specificity of the VHH is isolated and used to produce VHH that is not associated with said carrier. There are a variety of ways in which such VHH may be produced. Thus in a preferred embodiment, a method of the invention further comprises producing said antigen specific VHH. Since a preferred embodiment of the invention is concerned with VHH generated against antigens derived from proteins that are associated with the formation of proteinaceous aggregates, a preferred embodiment of the invention provides a method for producing an antigen specific VHH wherein said antigen is derived from a protein that is associated with the formation of proteinaceous aggregates. Such VHH will further be referred to as aggregation VHH. Thus in a preferred embodiment the invention provides a method of the invention further comprising producing said antigen specific aggregation VHH.

As a method of the invention is particularly suited to select and preferably screen VHH that are capable of at least reducing the formation of aggregates comprising said protein, the invention in a preferred embodiment provides a method of the invention further comprising determining whether a selected aggregation VHH is capable of at least reducing the formation of aggregates comprising said protein. This can be done using a system that promotes the formation of aggregates. Such a system typically, though not necessarily, involves the presence of cells producing the protein that aggregates. In case the protein produces extracellular aggregates one can provide the test VHH to the culture medium of the cells producing the protein. Alternatively, the VHH is produced by cells in the system. This is typically, though not necessarily the same cell as that produce said protein. When the protein produces intracellular aggregates, the VHH can also be provided to the culture medium of the cells producing the protein. This typically requires that the test VHH is taken up by the cells. This can be achieved by linking said VHH with a cell penetrating peptide. Non-limiting examples of such cell penetrating peptides are penetratin, Tat-fragment (48-60), Transportan and amphilic model peptide (for these and additional examples see Lindgren et al; 2000: TiPS Vol 21: pp 99-103).

Another method for introducing said VHHs into a target cell is to construct a bi-head VHH consisting of a VHH recognizing a specific receptor protein on the surface of the target cell and a VHH with the functional property to prevent aggregation or to dissolve existing aggregates (Roovers and van Bergen Henegouwen in preparation).

In a preferred embodiment the invention provides a VHH as specified in table 2, table 5.3, table 10, table 13 or table 14 or a derivative thereof. In a preferred embodiment said VHH further comprises another VHH. Said further VHH preferably comprises the same sequence as said first VHH. In another embodiment said further VHH comprises a VHH that can translocate via the blood brain barrier to the brain. In a preferred embodiment the invention provides a molecule comprising at least two VHH. In a preferred embodiment a first and a second of said at least two VHH comprises the same CDR amino acid sequence preferably a CDR amino acid as depicted in table 2, table 5.3, table 10, table 13 or table 14 or a derivative thereof. In a preferred embodiment a first and a second of said at least two VHH comprises the same amino acid sequence preferably an amino acid as depicted in table 2, table 6.3, table 10, table 13 or table 14 or a derivative thereof. In another embodiment said further VHH comprises a VHH that can translocate via the blood brain barrier to the brain, preferably a VHH according to table 12. The invention provides a heavy chain variable domain antibody (VHH) comprising at least a CDR1, CDR2 or CDR3 sequence as depicted in table 2, table 5.3, table 10, table 13 or table 14. Preferably said VHH comprises a sequence as depicted in table 2, table 5.3, table 10, table 13 or table 14 or a derivative thereof. Preferably said VHH comprises a sequence as depicted in table 2, table 5.3, table 10, table 13 or table 14, comprising a hallmark amino acid residue selected from the amino acids depicted for the corresponding position in table 3, preferably in the combination as depicted in table 5.2. In a preferred embodiment said VHH comprises a sequence as depicted in table 2, table 6.3, table 10, table 13 or table 14, comprising an amino acid residue selected from the amino acids depicted for the corresponding position in table 6 for framework 1, table 7 for framework 2, table 8 for framework 3 and/or table 9 for framework 4. Preferably said amino acid residue of table 3, table 6, table 7, table 8 or table 9 replaces the corresponding amino acid of table 2, table 5.3, table 10, table 13 or table 14. The invention further provides a VHH according to the invention, comprising an amino acid residue depicted for camelid VHH in any of table 6-9. Preferably a VHH of the invention comprises between 1 and 5 amino acid substitutions compared to the sequence as depicted in table 2, table 5.3, table 10, table 13 or table 14. The VHH as described in this paragraph are preferred VHH of the invention and can be modified and used in methods, cells and production and selection methods described herein. The invention further provides nucleic acid encoding said VHH, cells comprising said VHH and vectors and expression vectors as described herein. VHH alone or in tandem may further be provided with additional amino acid sequences as described herein, preferably a is provided with a signal sequence for directing the VHH to a specific location in a cell as described herein. In a preferred embodiment the invention provides a tandem VHH (Bi-head), comprising to VHH of the same specificity and/or a tandem VHH comprising a VHH of the invention joined to a VHH that can translocate via the blood brain barrier to the brain, preferably a VHH of table 12.

As transport over at least the cellular membrane is an additional property; it is preferred in these cases to produce the test VHH in the same cell that produce the intracellular aggregate. In a particularly preferred embodiment, said method further comprises determining whether said VHH is capable of at least decreasing the size of formed aggregates comprising said protein. Decrease in size can be due to dissolvement of the aggregate as a result of the binding of the VHH. Alternatively, it can be the result of attracting dissolving functions to the aggregate, or it can be the result of inhibition of the formation such that dissolving functions that were already present are no longer counteracted by de novo formation, or it can be a combination of the mentioned effects. Non-limiting examples of dissolving functions are proteases, proteasomes and chaperones.

The invention further provides a VHH obtainable by a method of the invention. Preferably, said VHH is specific for a protein encoded by a gene of table 1. In a particularly preferred embodiment said VHH is an aggregation VHH. Especially preferred are VHH specific for proteins involved in skeletal and cardiac muscle disorders: emerin (EMD), nuclear poly(A)-binding protein (PABPN1), tropomyosin-1 (TPM1) and actin (ACTA1).

Emerin (EMD) is a ubiquitously expressed member of the nuclear lamina-associated protein family. Mutations in the EMD gene result in X-linked Emery-Dreyfuss muscular dystrophy (EDMD). This myopathy is characterized by early contractures, progressive muscle weakness and wasting of the humero-peroneal musculature and cardiac conduction defects. Despite progress in understanding the functions of emerin [b11][Bengtsson L & Wilson K L 2004, Curr Opin Cell Biol 16, 73-79] EDMD is not yet understood at the molecular level. Skeletal muscle alpha-actin (ACTA1) forms thin filaments for which mutations are associated with two different muscle diseases [b17] [Nowak K J et al. 1999, Nature Genet. 23, 208-212]: congenital myopathy with excess of thin myofilaments, also known as actin myopathy, [b18][Goebel H H et al. 1997, Neuromuscul. Disord 7, 160-168] and nemaline myopathy. The disease is characterized by structural abnormalities of muscle fibers and variable degrees of muscle weakness. Finally, tropomyosin-1 (TPM1) is the striated muscle isoform of tropomyosin. Tropomyosins exist in different isoforms and associate with actin filaments in myofibrils and stress fibers. Tropomyosin-1 is an important component of muscle thin filaments and missense mutations cause familial hypertrophic cardiomyopathy CMH3 [b19] [Thierfelder L et al 1994, Cell 77, 701-712]

In a particularly preferred embodiment said protein comprises a protein that is in aggregation associated disease is associated with nucleotide expansion of the coding region. In a preferred embodiment said protein comprises nuclear poly(A)binding protein 1 (PABPN1). PABPN1 is associated with oculopharyngeal muscular dystrophy (OPMD, MIM164300). OPMD is a late-onset disease, clinically characterized by slow progressive ptosis, dysphagia and limb girdle weakness [c1][Brais B, Cytogenet. Genome Res. 100, 252-560] OPMD is usually inherited as an autosomal dominant trait and caused by a trinucleotide repeat expansion in the coding region of the nuclear poly(A)-binding protein 1 (PABPN1) gene.[c2] Brais B et al. 1998, Nature Genet. 18, 164-167. The alanine stretch that is encoded by this trinucleotide sequence contains 10 alanines in the non-affected protein, but is expanded to 12-17 alanines in the mutant protein in autosomal dominant OPMD.

PABPN1 is ubiquitously expressed and is involved in poly(A)-tail synthesis and poly(A)—tail length-control [c3] Wahie E 1991, Cell 66, 759-768]. One of the pathological hallmarks of OPMD is the presence of PABPN1-containing fibril-like aggregates in 2-5% of myonuclei in affected muscle [c4-6][Calado A et al. 2000, Human Mol. Genet. 9, 2312-2328; Uyama E et al. 2000, Muscle Nerve 23, 1549-1554; Becher M W et al. 2000, Ann Neurol. 48, 812-815. The roles of the formation of the intranuclear inclusions in the progression of OPMD are poorly understood [c1, 7, 8] [Brais B, Cytogenet. Genome Res. 100, 252-560, Berciano M T et al 2004, Hum. Mol. Genet. 13, 829-838, Davies et al. 2005, Nature Med. 11, 672-677].

Recently, a doxycyline-based treatment for OMPD was proposed based on animal studies with transgenic mice [c8][Davies et al. 2005, Nature Med. 11, 672-677]. These mice develop OPMD-like muscle defects and show intranuclear aggregation of mutant PABPN1. Upon doxycycline treatment, the muscle defects improved and aggregate formation was reduced, suggesting a direct role for aggregate formation in OPMD pathogenesis.

PABPN1 aggregation has also been studied in cellular models using transient expression of wild type and expanded PABPN1. Aggregate formation was inhibited in these cellular models for OPMD by doxycycline, Congo red and over-expressed chaperones[c9-11] Abu-Baker et al 2003, 12, 2609-2623; Bao Y P et al 2002, 277 12263-12669; Bao Y P et al 2004 J. Med. Genet. 41, 47-51]. These studies also resulted in increasing knowledge of the toxicity of the intranuclear inclusions, proteins and nucleic acids included in the formed inclusions, and the dynamic nature of the intranuclear inclusions [c12] Fan X. et al 2001, Hum. Mol. Genet. 10, 2341-2351. For example, it was shown that reduction of aggregate formation leads to increased cell survival.

Despite the effectiveness of some of these aggregate reducing agents, none of them is specific for PABPN1 aggregates. Instead, many of these agents were tested in analogy to other protein aggregation conditions and operate through poorly understood mechanisms. Antigen-specific approaches to reduce aggregation have also been described. By intracellular expression of single-chain Fv (scFv) [c13] Davies et al. 2005, Nature Med. 11, 672-677] and VL VHH [c14] [Colby D W et al. 2004, Proc. Natl. Acad. Sci. 101, 17616-17121] it was possible to inhibit huntingtin exon 1 aggregation in cellular models for Huntingon's disease [13, 14?] Davies et al. 2005, Nature Med. 11, 672-677; Colby D W et al. 2004, Proc. Natl. Acad. Sci. 101, 17616-17121].

Presently a method of the invention was used to isolate a VHH against PABPN1. Using the VHH we could inhibit intranuclear inclusion formation in OPMD cell models. Using a combination of selections for VHH carriers, we obtained different sets of VHH. By intracellular expression of some of these VHH we were able to inhibit aggregation in situ in a dose dependent manner. Experiments with serial expression of mutant PABPN1 and PABPN1-specific VHH prove that even existing aggregates are cleared.

As VHH that are produced intracellularly need to act at particular location in the cell (or outside the cell), VHH of the invention preferably comprise a signal sequence for directing the VHH to a specific location in a cell. There are many different signal sequences. Signal sequences for a particular location in a cell typically share a common structural and/or amino acid sequence motif. In a preferred embodiment said signal sequence directs said VHH to the nucleus, the endoplasmic reticulum and/or the exterior of a cell. Said signal sequence is preferably provided to the VHH. The signal sequence can be provided directly to the VHH, however, typically the signal sequence is provided by expressing a fusion protein comprising the signal sequence and the VHH.

Antibody technology is currently very well developed. Many different manipulation techniques are available to the person skilled in the art. For instance, the antigen specificity of a VHH of the present invention can be transferred to another VHH by grafting the CDR3 sequences. Thus in one embodiment, the invention provides a VHH comprising a CDR3 sequence of a VHH depicted in table 2 or table 5.3 or table 10. In a preferred embodiment, said VHH comprises the CDR1, CDR2 and CDR3 sequence of a VHH depicted in table 2 or table 5.3. In a particularly preferred embodiment, the invention provides a VHH comprising a sequence as depicted in table 2 or table 5.3.

The invention further provides a nucleic acid molecule encoding a VHH of the invention. In another embodiment the invention provides a recombinant and/or isolated cell provided with a nucleic acid encoding a VHH of the invention. In yet another embodiment the invention provides a recombinant and/or isolated cell comprising a VHH of the invention. Preferably, said cell is provided with said VHH. Further provides is a recombinant and/or isolated cell according to the invention, provided with a nucleic acid encoding a VHH of the invention.

Furthermore the invention provides a bi-head VHH that consists of a VHH capable of passing the blood-brain-barrier and a VHH with the functionality to prevent or dissolve extracellular aggregates in the brain.

In yet another embodiment the invention provides an isolated and/or recombinant gene delivery vehicle comprising a nucleic acid of the invention. The invention further provides a method for producing a VHH of the invention, comprising providing a cell with a nucleic acid of the invention and culturing??? said cell to allow production of said VHH.
a) The term ‘antigenic determinant’ refers to the epitope on the antigen recognized by the antigen-binding molecule (such as a VHH or a polypeptide of the invention) and more in particular by the antigen-binding site of said molecule
b) An amino acid sequence (such as a VHH, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
c) The term “specificity” refers to the number of different types of antigens or antigenic determinants to which a particular antigen-binding molecule or antigen-binding protein (such as a VHH or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a VHH or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the VHH and/or polypeptides of the invention) will bind with a dissociation constant (K_{D}) of 10⁻⁵ to 10⁻¹² moles/Liter or less. Any K_{D} value greater than 10⁻⁴ liters/mol is generally considered to indicate non-specific binding. Preferably, a VHH or polypeptide of the invention will bind to the desired antigen with an affinity less than 500 nM, preferably less than 200 nM, more preferably less than 10 nM, such as less than 500 pM;
d) As further described herein, the amino acid sequence and structure of a VHH can be considered—without however being limited thereto—to be comprised of four framework regions or “FR's”, which are referred to in the art and herein as “Framework region 1” or “FR1”; as “Framework region 2” or “FR2”; as “Framework region 3” or “FR3”; and as “Framework region 4” or “FR4”, respectively; which framework regions are interrupted by three complementary determining regions or “CDR's”, which are referred to in the art as “Complementarity Determining Region 1” or “CDR1”; as “Complementarity Determining Region 2” or “CDR2”; and as “Complementarity Determining Region 3” or “CDR3”, respectively;
e) As also further describe herein, the total number of amino acid residues in a VHH can be in the region of 110-120, is preferably 112-115, and is most preferably 113. It should however be noted that parts, fragments, analogs or derivatives (as further described herein) of a VHH are not particularly limited as to their length and/or size, as long as such parts, fragments, analogs or derivatives meet the further requirements outlined herein and are also preferably suitable for the purposes described herein; The amino acid residues of a VHH are numbered according to the general numbering for V_{H} domains given by Kabat et al. (“Sequence of proteins of immunological interest”, US Public Health Services, NIH Bethesda, Md., Publication No. 91), as applied to V_{HH} domains from Camelids in the article of Riechmann and Muyldermans, referred to above (see for example FIG. 2 of said reference). According to this numbering, FR1 of a VHH comprises the amino acid residues at positions 1-30, CDR1 of a VHH comprises the amino acid residues at positions 31-36, FR2 of a VHH comprises the amino acids at positions 36-49, CDR2 of a VHH comprises the amino acid residues at positions 50-65, FR3 of a VHH comprises the amino acid residues at positions 66-94, CDR3 of a VHH comprises the amino acid residues at positions 95-102, and FR4 of a VHH comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.].

Alternative methods for numbering the amino acid residues of V_{H} domains, which methods can also be applied in an analogous manner to V_{HH} domains from Camelids and to VHH, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise; and
g) The Figures, Sequence Listing and the Experimental Part/Examples are only given to further illustrate the invention and should not be interpreted or construed as limiting the scope of the invention and/or of the appended claims in any way, unless explicitly indicated otherwise herein.

For a general description of heavy chain antibodies and the variable domains thereof, reference is inter glia made to the following references, which are mentioned as general background art: WO 94/04678 (=EP 656 946), WO 96/34103 (=EP 0 822 985) and WO 97/49805, all by the Vrije Universiteit Brussel; WO 97/49805 by Vlaams Interuniversitair Instituut voor Biotechnologie; WO 94/25591 (=EP 0 698 097) and WO 00/43507 by Unilever N. V.; WO 01/90190 by the National Research Council of Canada; WO 03/025020 (=EP 1 433 793) by the Institute of Antibodies; WO 04/062551, WO 04/041863, WO 04/041865, WO 04/041862; Hamers-Casterman et al, Nature, Vol. 363, p. 446 (1993); Riechmann and Muyldermans, Journal of Immunological Methods, 231 (1999), p. 25-38; Vu et al., Molecular Immunology, Vol. 34, No. 16-17, p. 1121-1131 (1997); Nguyen et al., EMBO J., Vol. 19, No. 5, 921-930 (2000); Arbabi Ghahroudi et al., FEBS Letters 414 (1997) 521-526; van der Linden et al., J. Immunological Methods, 240 (2000), 185-195; Muyldermans, Reviews in Molecular Biotechnology 74 (2001), 277-302; Nguyen et al., Advances in Immunology; Vol. 79 (2001); 261; as well as some of the further references mentioned herein.

In accordance with the terminology used in the above references, the variable domains present in naturally occurring heavy chain antibodies will also be referred to as “V_{HH domains}”, in order to distinguish them from the heavy chain variable domains that are present in conventional 4-chain antibodies (which will be referred to hereinbelow as “V_{H domains}”) and from the light chain variable domains that are present in conventional 4-chain antibodies (which will be referred to hereinbelow as “V_{L domains}”).

As mentioned in the prior art referred to above, V_{HH} domains have a number of unique structural characteristics and functional properties, which make isolated V_{HH} domains (as well as VHH based thereon, which share these structural characteristics and functional properties with the naturally occurring V_{HH} domains) and proteins containing the same highly advantageous for use as functional antigen-binding domains or proteins. In particular, and without being limited thereto, VHH domains (which have been “designed” by nature to functionally bind to an antigen without the presence of and without any interaction with, a light chain variable domain) and VHH can function as a single, relatively small, functional antigen-binding structural unit, domain or protein. This distinguishes the V_{HH} domains from the V_{H} and V_{L} domains of conventional 4-chain antibodies, which by themselves are generally not suited as antigen-binding proteins or domains, but need to be combined in some form or another to provide a functional antigen-binding unit (as in for example conventional antibody fragments such as Fab fragments; or in ScFv's fragments, which consist of a V_{H} domain covalently linked to a V_{L} domain).

Because of these unique properties, the use of V_{HH} domains and VHH as antigen-binding proteins or antigen-binding domains (i.e. as part of a larger protein or polypeptide) offers a number of significant advantages over the use of conventional V_{H} and V_{L} domains, scFv's or conventional antibody fragments (such as Fab- or F(ab)₂-fragments):
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
V_{HH} domains and VHH can be expressed from a single gene and require no post-translational folding or modifications;
V_{HH} domains and VHH can easily be engineered into multivalent and multispecific formats (as further discussed herein);
V_{HH} domains and VHH are highly soluble and do not have a tendency to aggregate (as with the mouse-derived antigen-binding domains” described by Ward et al., Nature, Vol. 341, 1989, p. 544);
V_{HH} domains and VHH are highly stable to heat, pH, proteases and other denaturing agents or conditions;
V_{HH} domains and VHH are easy and relatively cheap to prepare, even on a scale required for production. For example, V_{HH} domains, VHH and proteins/polypeptides containing the same can be produced using microbial fermentation (e.g. as further described below), and do not require the use of mammalian expression systems, as with for example conventional antibody fragments;
V_{HH} domains and VHH are relatively small compared to conventional 4-chain antibodies and antigen-binding fragments thereof, and therefore show high(er) penetration into tissues (including but not limited to solid tumors) than such conventional 4-chain antibodies and antigen-binding fragments thereof;
V_{HH} domains and VHH can show so-called cavity-binding properties, and can therefore also access targets and epitopes not accessible to conventional 4-chain antibodies and antigen-binding fragments thereof. For example, it has been shown that V_{HH} domains and VHH can inhibit enzymes (see for example WO 97/49805; Transue et al., Proteins: structure, function, genetics, 32: 516-622 (1998; Lauwereys et al., EMBO J., Vol. 17, No. 13, p. 3612-3520). A particular useful property of VHH's is that when intracellularly expressed it folds correctly in the absence of S—S formation between Cys (22) and Cys (92).

As mentioned above, the invention generally relates to VHH directed against, as well as to polypeptides comprising or essentially consisting of one or more of such VHH, that can be used for the prophylactic, therapeutic and/or diagnostic purposes described herein.

As also further described herein, the invention further relates to nucleic acids encoding such VHH and polypeptides, to methods for preparing such VHH and polypeptides, to host cells expressing or capable of expressing such VHH or polypeptides, to compositions comprising such VHH, polypeptides, nucleic acids or host cells, and to uses of such VHH, polypeptides, nucleic acids, host cells or compositions.

Generally, it should be noted that the term VHH as used herein in its broadest sense is not limited to a specific biological source or to a specific method of preparation. For example, as will be discussed in more detail below, the VHH of the invention can generally be obtained: (1) by isolating the Vim domain of a naturally occurring heavy chain antibody; (2) by expression of a nucleotide sequence encoding a naturally occurring V_{HH} domain; (3) by “humanization” (as described herein) of a naturally occurring V_{HH} domain or by expression of a nucleic acid encoding a such humanized V_{HH} domain; (4) by “camelization” (as described herein) of a naturally occurring V_{H} domain from any animal species, and in particular a from species of mammal, such as from a human being, or by expression of a nucleic acid encoding such a camelized V_{H} domain; (5) by “camelisation” of a “domain antibody” or “Dab” as described by Ward et al (supra), or by expression of a nucleic acid encoding such a camelized V_{H} domain; (6) by using synthetic or semi-synthetic techniques for preparing proteins, polypeptides or other amino acid sequences known per se; (7) by preparing a nucleic acid encoding a VHH using techniques for nucleic acid synthesis known per se, followed by expression of the nucleic acid thus obtained; and/or (8) by any combination of one or more of the foregoing. Suitable methods and techniques for performing the foregoing will be clear to the skilled person based on the disclosure herein and for example include the methods and techniques described in more detail herein.

One preferred class of VHH corresponds to the V_{HH} domains of naturally occurring heavy chain antibodies directed against. As further described herein, such V_{HH} sequences can generally be generated or obtained by suitably immunizing a species of Camelid with (i.e. so as to raise an immune response and/or heavy chain antibodies directed against), by obtaining a suitable sample from said Camelid (such as a blood sample, serum sample or sample of B-cells), and by generating V_{HH} sequences directed against starting from said sample, using any suitable technique known per se. Such techniques will be clear to the skilled person and/or are further described herein.

Alternatively, such naturally occurring V_{HH} domains against can be obtained from non-immunized libraries of Camelid V_{HH} sequences, for example by screening such a library against or at least one part, fragment, antigenic determinant or epitope thereof using one or more screening techniques known per se. Such libraries and techniques are for example described in WO 99/37681, WO 01/90190, WO 03/025020 and WO 03/035694. Alternatively, improved synthetic or semi-synthetic libraries derived from naïve V_{HH} libraries may be used, such as V_{HH} libraries obtained from naïve V_{HH} libraries by techniques such as random mutagenesis and/or CDR shuffling, as for example described in WO 00/43507.

Yet another technique for obtaining V_{HH} sequences directed against involves suitably immunizing a transgenic mammal that is capable of expressing heavy chain antibodies (i.e. so as to raise an immune response and/or heavy chain antibodies directed against), obtaining a suitable sample from said transgenic mammal (such as a blood sample, serum sample or sample of B-cells), and then generating V_{HH} sequences directed against starting from said sample, using any suitable technique known per se. For example, for this purpose, the heavy chain antibody-expressing mice and the further methods and techniques described in WO 02/085945 and in WO 04/049794 can be used.

A particularly preferred class of VHH of the invention comprises VHH with an amino acid sequence that corresponds to the amino acid sequence of a naturally occurring V_{HH} domain, but that has been “humanized”, i.e. by replacing one or more amino acid residues in the amino acid sequence of said naturally occurring V_{HH} sequence by one or more of the amino acid residues that occur at the corresponding position(s) in a V_{H} domain from a conventional 4-chain antibody from a human being (e.g. indicated above). This can be performed in a manner known per se, which will be clear to the skilled person, for example on the basis of the further description herein and the prior art on humanization referred to herein. Again, it should be noted that such humanized VHH of the invention can be obtained in any suitable manner known per se (i.e. as indicated under points (1)-(8) above) and thus are not strictly limited to polypeptides that have been obtained using a polypeptide that comprises a naturally occurring V_{HH} domain as a starting material.

Another particularly preferred class of VHH of the invention comprises VHH with an amino acid sequence that corresponds to the amino acid sequence of a naturally occurring V_{I}/domain, but that has been “camelized”, i.e. by replacing one or more amino acid residues in the amino acid sequence of a naturally occurring V_{H} domain from a conventional 4-chain antibody by one or more of the amino acid residues that occur at the corresponding position(s) in a V_{HH} domain of a heavy chain antibody. This can be performed in a manner known per se, which will be clear to the skilled person, for example on the basis of the further description herein. Reference is also made to WO 94/04678. Such “camelizing” substitutions are preferably inserted at amino acid positions that form and/or are present at the V_{H}-V_{L}, interface, and/or at the so-called Camelidae hallmark residues, as defined herein (see for example also WO 94/04678). Preferably, the V_{H} sequence that is used as a starting material or starting point for generating or designing the camelized VHH is preferably a V_{H} sequence from a mammal, more preferably the Vu sequence of a human being, such as a V_{H}3 sequence. However, it should be noted that such camelized VHH of the invention can be obtained in any suitable manner known per se (i.e. as indicated under points (1)-(8) above) and thus are not strictly limited to polypeptides that have been obtained using a polypeptide that comprises a naturally occurring V_{H} domain as a starting material.

For example, again as further described herein, both “humanization” and “camelization” can be performed by providing a nucleotide sequence that encodes a naturally occurring V_{HH} domain or V_{H} domain, respectively, and then changing, in a manner known per se, one or more codons in said nucleotide sequence in such a way that the new nucleotide sequence encodes a “humanized” or “camelized” VHH of the invention, respectively. This nucleic acid can then be expressed in a manner known per se, so as to provide the desired VHH of the invention. Alternatively, based on the amino acid sequence of a naturally occurring V_{HH} domain or V_{H} domain, respectively, the amino acid sequence of the desired humanized or camelized VHH of the invention, respectively, can be designed and then synthesized de novo using techniques for peptide synthesis known per se. Also, based on the amino acid sequence or nucleotide sequence of a naturally occurring V_{HH} domain or V_{H} domain, respectively, a nucleotide sequence encoding the desired humanized or camelized VHH of the invention, respectively, can be designed and then synthesized de novo using techniques for nucleic acid synthesis known per se, after which the nucleic acid thus obtained can be expressed in a manner known per se, so as to provide the desired VHH of the invention.

Other suitable ways and techniques for obtaining the VHH of the invention and/or nucleic acids encoding the same, starting from naturally occurring V_{H} sequences or preferably V_{HH} sequences, will be clear from the skilled person, and may for example comprise combining one or more parts of one or more naturally occurring V_{H} sequences (such as one or more FR sequences and/or CDR sequences), one or more parts of one or more naturally occurring V_{HH} sequences (such as one or more FR sequences or CDR sequences), and/or one or more synthetic or semi-synthetic sequences, in a suitable manner, so as to provide a VHH of the invention or a nucleotide sequence or nucleic acid encoding the same.

### EXAMPLE 1

### Materials and Methods

### Nonimmune VHH Library

For VHH selections a large llama-derived nonimmune VHH library was used (Hermans et al., in preparation) which was kindly provided for this study by Unilever Research Vlaardingen, The Netherlands. This library with a clonal diversity of 5×10⁹ was constructed with RNA extracted from peripheral blood lymphocytes that were collected from the blood of 8 non-immunized llama's. The phage display library was generated essentially as described before [20][WO 99/376811].

### Production and Purification of Recombinant Proteins and VHH

cDNA encoding amino acids 1-179 of emerin and full-length actin and tropomyosin-1 cDNA's were PCR amplified from a total human muscle cDNA preparation, with primers EMERINFBAM: 5′-CGCGGATCCATGGACAACTACGCAGATCTT-3′, EMERINFBAM: 5′-CCGCCCTCGAGGTCCAGGGAGCTCCTGGAGGC-3′, ACT1: 5′-CGCGGATCCTGCGACGAAGACGAGACCACC-3′, ACT2: 5′-CGCAAGCTTGGAAGCATTTGCGGTGGACGAT-3′ and TP1: 5′-CGCGGATCCGACGCCATCAAGAAGAAGATG-3′, TP2: 5′-CGCAAGCTTGCATGGAAGTCATATCGTTGAG-3′, respectively, in 35 cycles of 95° C. for 1 min, 62° C. for 1 min and 72° C. for 2 min, following an initial denaturation step of 5 min at 95° C. All sets of primers introduced a 5′-BamHI and a 3′-HindIII or XhoI restriction site that allowed directed in-frame cloning in pET28a expression vector (EMD Biosciences, Novagen). In a similar way an expression construct for full-length PABPN1 was prepared. Expression constructs were sequence verified (LGTC, The Netherlands). Recombinant antigens carrying a T7•Tag and a His•Tag, were produced in E. coli BL-21(DE3)-RIL cells (Stratagems), according to standard protocols and were subsequently purified by IMAC according to the instructions of the manufacturer (Clontech). Purified recombinant antigens were dialyzed against PBS at 4° C. For downstream applications, all antigen concentrations were adjusted to 10 μg/ml. Single-batch antigens were used throughout the selection and screening process.

VHH were purified from periplasmic fractions of TG1 E. coli cells carrying the phagemids of interest. An overnight culture of a single clone was used to inoculate a new culture in 1:100 dilution. Bacteria were grown to an OD₆₀₀ of 0.5-0.7 whereafter VHH production was induced with the addition of IPTG (ICN) to a final concentration of 1 mM, for 4-5 hours. Bacterial pellets were resuspended in 1/50 or 1/25 of the initial culture volume of 1 mM EDTA-1M NaCl in PBS pH7.4, by gentle rotation at 4° C. for 1-2 hours. Hexahistidine-tagged VHH were purified from the supernatants by IMAC as above.

Selection of VHH Het is logischer om nu eerst verder to gaan met wat op pag 70 en bovenaan pag 71 staat van de PDF.Immers het gaat bier omde beschrijving van de uitgangsmaterialen.

Antigen presentation during selections with the phage library was performed in successive and alternating modes of passive antigen adsorption and antigen capturing with an anti-tag monoclonal antibody. For antigen capturing, polystyrene 96-well plates (Maxisorp, NUNC Denmark) were coated for twelve hours at 4° C. with 100 μl of 10 μg/ml anti-T7 tag monoclonal antibody (Novagen). Plates were rinsed three times with PBS, then blocked for 30 minutes at room temperature with 4% skimmed milk in PBS, rinsed once again with PBS and 100 μl of the antigen of interest in a concentration of 10 μg/ml, was added in 0.1% BSA in PBS. Plates were thereafter incubated for 2 hours at room temperature, with vigorous agitation at 1000 rpm on an ELISA shaker. For passive adsorption of the antigen (direct coating) 100 μl of 10 μg/ml protein of interest was used. Following three washes with PBS, 100 μl of the phage library mix consisting of approximately 10¹¹ phage and 20% normal mouse serum (Sigma-Aldrich, The Netherlands) in 2% skimmed milk in PBS and was added to each well and incubated for 2 hours as previously. Plates were washed 15 times with PBST (0.05% Tween-20) while every fifth wash they were placed on an elisa shaker at 1000 prm, for 10 minutes and finally rinsed 3 times with PBS. In all cases, bound phage were eluted with 100 μl of 100 mM solution of triethylamine, during a 10 minutes incubation and were subsequently neutralized with 50 μl 1M Tris pH7.5. For epitope-masking selections [[Verheesen P et al 2006 in press] against PABPN1, 10 μg/ml VHH3F5 was coated to polystyrene plates and PABPN1 was captured as described before. Capturing with antigen specific antibody fragments blocks off antigenic sites and favors selection against other epitopes on the same antigen. Half of the eluted phage was used to infect mid-log phase E. coli TG1. Dilutions from each infection mix were used to calculate the outputs after each round of selection and enrichments were calculated by division of the round 2 output/input ratios by the round 1 output/input ratios. Phage were prepared from E. coli as described [[Frenken L G et al 2000, J. Biotechnol 78, 11-21].

### SDS-PAGE and Western Blot Analysis

Proteins were separated by SDS-PAGE followed by Coomassie Brilliant Blue staining or transfer to PVDF Western blotting membranes (Roche Diagnostics, Almere, The Netherlands) using the Mini-PROTEAN 8 system for gel electrophoresis and the Mini Trans-Blot Cell for blotting of the proteins (Bio-Rad Laboratories, Hercules, Calif., USA). After protein transfer, membranes were blocked overnight in 4-5% skimmed milk in PBS at 4° C. or incubated two times in pure methanol and then allowed to dry (Liu B et al. 2002 J. Mol. Biol. 315, 1063-1073]. A dilution of phage (typically to 10⁷ cfu/ml) or VHH (typically 50 nM-1 μM) were incubated in 2-5% skimmed milk in PBS for 1-2 h at RT or overnight at 4° C. c-Myc tagged VHH were detected by anti-c-Myc monoclonal antibody (kindly provided by P. W. Hermans, Biotechnology Application Centre BV, Bussum, The Netherlands) and 5,000-fold diluted anti-mouse horseradish peroxidase (HRP) conjugate (Jackson ImmunoResearch, West Grove, USA). For phage detection a 10,000-fold dilution of a HRP-conjugated monoclonal antibody which binds to the phage coat protein was used (Amersham Biosciences, Uppsala, Sweden).

### Evaluation of the Selection Process

Polyclonal phage from each round of selection were used to monitor the progress of the selection by 1D-gel electrophoresis and Western blotting before isolating and characterizing individual clones. Dilutions of phage (1:1000) in 5% skimmed milk in PBS were tested for binding to their associated antigens with blotted recombinant proteins onto PVDF membranes, utilizing the above mentioned systems and following the protocol as described by Liu et al. [24][Liu B et al. 2002 3. Mol. Biol. 315, 1063-1073] [.

### Screening of Positive Clones by Phage ELISA

Overnight cultures of single colonies from second selection rounds, grown in a U-bottom 96-well plate (NUNC, Denmark) at 37° C., in 2×TY medium containing 100 μg/ml ampicillin and 2% glucose, were used to inoculate new cultures with 0.1% glucose. Bacteria were grown to an O₆₀₀ of 0.5 and then a mixture of VCSM13 helper phage, kanamycin and ampicillin was added to the cultures to final concentrations of 10⁹ CFU/ml helper phage, 25 μg/ml kanamycin and 100 μg/ml ampicillin. Bacteria were further grown overnight with shaking at 220 rpm at 37° C.

For ELISA, Maxisorp plates (NUNC, Denmark) were coated with 100 μl of 10 μg/ml of each antigen of interest, first for 30 minutes at room temperature shaking at 1000 rpm and then overnight standing at 4° C. The following day the plates were blocked for 1 hour with 5% skimmed milk in PBS and 50 μl of phage containing supernatants was added to the wells. After two hours of incubation at room temperature at 1000 rpm, the plates were rinsed three times with PBST (0.05% Tween-20) and three times with PBS. To detect the antigen phage-antibody interaction, a 1:10,000 dilution in 5% milk-PBS solution of anti-M13 monoclonal antibody conjugated to horseradish peroxidase (Amersham Pharmacia) was added to the wells, and incubated for one hour. Following three washes with PBS, the reacting complex was visualized by adding 100 μl of OPD solution containing 3.7 mM o-phenylenediamine (ICN Biomedicals), 50 mM Na₂HPO₄, 25 mM citric acid and 0.03% H₂O₂. The enzymatic reaction was stopped by adding H₂SO₄ to a final concentration of 300 mM. Colour intensities were quantified by measuring the OD₄₉₀ in an ELISA plate reader (BioTek).

### Screening of Clones by DNA Fingerprinting

To generate DNA fingerprints, PCR reactions were performed with primers M13Rev: 5′-CAGGAAACAGCTATGAC-3′ and MPE25: 5′-TTTCTGTATGGGGTITTTTGCTA-3′ using as a template 1 μl of the glycerol stocks contained in a panel of 96 individual colonies originating from the second round of selection. A PCR Master mix consisting of 1× SuperTaq buffer (HT Biotechnology, The Netherlands), 1.25 mM dNTPs, 12 pM of each primer and 0.5 Up er reaction of Silverstar polymerase (Eurogentec) was dispensed into 20 μl aliquots in a 96-well PCR plate. The amplification was performed in 35 cycles of 94° C., 1′; 55° C. 1′ and 72° C., 1.5′ following an initial denaturation step for 10′ at 94° C. After the PCR reaction, 5 μl of the amplified products were digested in a total volume of 25 μl with HinfI and analyzed on a 3% (w/v) agarose gel in TBE buffer. Clones that showed binding to the recombinant antigens by ELISA and yielded different restriction patterns with fingerprint analysis were sequenced using either of the primers M13Rev or MPE25 (LGTC, The Netherlands).

Screening of VHH Clones with Different Finger Prints by Expression of these Clones in E. coli and S. cerevisiae.

As proper folding is an essential property of the VHHs to combat the aggregation diseases, we tested the folding of these VHHs in E. coli and in S. cerevisiae. Cloning of VHHs in these cells have been described in the literature [[Frenken L G et al. 2000, J. Biotechnol. 78, 11-20]]. Evaluation of the amount of soluble VHHs in the periplasmic space of E. coli and of soluble VHHs in the culture medium of S. cerevisiae as function of the biomass of these cultures provide a good indication of proper folding. The screening criterium is simply the yield of VHH divided by the biomass, compare e.g. [Tomassen Y E et al 2002, Enzyme and Micobiol. Technology 30, 273-278]. Normally only one clone out of 4 will pass set criterium.

### Immunoprecipitation

Immunoprecipitation is another step in the screening protocol. The step is introduced to ensure that the conformation of the antigen related to the disease is really recognized by the candidate VHH.

HeLa cells were cultured according to standard protocols. Cells were harvested by scraping in 1×NP-40 buffer (50 mM Tris-HCl pH8, 150 mM NaCl, 1% (v/v)NP-40, 1× complete protease inhibitors (Roche)). The cell suspension was sonicated until clarity. VHH3F5 was added (final concentration 100 nM) and incubated overnight at 4° C. with head-over-head rotation. Protein A sepharose was added to 10% (v/v) and incubated for 1 h at 4° C. with head-over-head rotation. The resin was washed 3 times with 1×NP-40 buffer and once with 50 mM Tris-HCl pH8. Bound proteins were eluted with 100 mM glycine pH2.5 by head-over-head rotation for 5 min, and neutralized with 1M Tris.

### Immunofluorescence Microscopy

For immunocytochemistry, control fibroblasts and LMNA^{-/-} fibroblasts (kindly provided by Dr. B. van Engelen, Nijmegen, Netherlands; [25] [Muchir A et al. 2003, Exp. Cell. Res. 291, 352-362]) were grown on coverslips in F12 medium supplemented with 10% FCS and penicillin/streptomycin to prevent bacterial growth. The cells were rinsed once with PBS and fixed with 10% v/v formalin (J. T. Baker) for 10 minutes at RT. Permeabilization of cells was performed with 0.1% Triton in PBS for 10 minutes at RT. After blocking with 50 mM glycine in PBS for 10 minutes at RT and 1% BSA in PBS for 30 minutes at RT, primary antibodies were diluted in 1% BSA in PBS to 0.05-1 μM when VHH were used and incubated for 2 h at RT. Bound VHH were detected with a serial combination of anti-VSV monoclonal antibody (kindly provided by Dr J. Franssen, Nijmegen, Netherlands) or anti-c-Myc monoclonal antibody and Alexa Fluor 488 goat anti-mouse IgG (Molecular Probes, Eugene, Oreg., USA) all incubated in 1% BSA in PBS for 1 h at RT. Rabbit anti-Lamin A polyclonal antibodies (Cell Signaling Technology, Beverly, Mass., USA) were diluted 1:35 in 1% BSA in PBS and incubated for 2 h at RT. Rabbit antibodies were detected with Alexa Fluor 594 goat anti-rabbit IgG (Molecular Probes). Labelling of F-actin was performed with Alexa Fluor 568 phalloidin according to the provided protocol (Molecular Probes, Invitrogen). Muscle thin cryosections, 6-8 μM, were cut on a cryotome (Shandon, USA), subsequently melted on SuperFrost Plus (Menzel Glazer) glass slides and sections were fixed by drying to the air. After two rinses with PBS, the sections were blocked with 1% BSA in PBS for at least 30 minutes and antibodies were incubated under the same conditions as described for immunocytochemistry.

### Results

### Defining the Optimal Selection Strategy

Full-length actin, tropomyosin-1, PABPN1 and the intranuclear domain of emerin were cloned in pET28a expression vectors. Recombinant proteins were produced at high levels upon induction in E. coli BL21 cells. Actin appeared in the insoluble protein fraction while tropomyosin-1, PABPN1 and emerin were soluble. All were affinity purified by means of their hexahistidine tags and yields up to 20 mg per litre culture were obtained.

For the VHH selections a large nonimmune library was used (Hermans et al., in preparation). In order to identify optimal conditions for selection, three modes of antigen presentation were evaluated. Firstly, two identical rounds of selection for recombinant emerin utilizing passive adsorption of the antigen on a polystyrene surface according to the biopanning procedure [23] [Marks J D et al. 1991, J. Mol. Biol. 222, 581-597] resulted in isolation of one monoclonal antibody fragment for which we could not prove binding to the native human protein. Secondly, two identical rounds of selection by capturing the T7-tagged antigen with monoclonal antibody against the T7-tag, resulted in enrichment for binders to the capture antibody whereas no enrichment for the antigen could be observed, despite competition with excess of irrelevant mouse IgG during phage incubation. In our third strategy, we therefore also investigated combinations of antigen immobilization in successive rounds of selection. For the first round, tagged antigen was captured with the mouse monoclonal antibody against the T7-tag. Phage output numbers up to 2×10⁴ were obtained and phage pools were prepared from these sublibraries. To further increase the probability of picking antigen-specific clones and to reduce the chance of obtaining antibody fragments recognizing the capture antibody, a second round of selection was performed with passive adsorption of the antigen on a polystyrene surface. Comparatively, we evaluated the reverse order of first passive adsorption followed by antigen capturing. Although with both orders of antigen immobilization increasing numbers of phage were retrieved in consecutive selection rounds, a more diverse set of VHH was obtained when the antigen was antibody-captured in the first round of selection followed by a second round of passive absorption, as illustrated by the sequences of the selected antibody fragments (Table 10). For both orders of antigen presentation, all clones with showed binding to the recombinant antigen and were genetically different based on their DNA fingerprint, were sequenced. Clones that were applicable for the detection of emerin in human muscle tissue sections (clones EME7E, EME2G and VHH14) were solely obtained by the selections with capturing of the antigen in the first round of selection. Therefore, selections for tropomyosin-1, actin, and PABPN1 were performed with this order of antigen immobilization, as well. Enrichments were determined after two rounds of selection as defined by the increase in proportion of input phage eluted from the antigen in successive rounds of selection. The calculated enrichments pointed towards successful selections and so the outputs of the selections were evaluated further.

### Monitoring the Selection Process

Whole phage particles displaying antibody fragments can be successfully used as primary antibodies for antigen detection on Western blots [24, 26][Liu B et al. 2002, J. Mol. Biol. 315, 1063-1073; Nissim A et al. 1994 EMBO J. 13, 692-698]. We explored the use of whole phage selection outputs as primary antibody source to monitor the selection progress. By Western blotting of recombinant antigens and incubation with polyclonal phage antibodies from successive rounds of selection an increase in signal intensity at the correct molecular weight of the recombinant emerin, tropomyosin-1 and actin was observed as selections proceeded (FIG. 1, panels a-c). After two rounds of selection emerin and actin could be specifically detected with polyclonal phage antibodies (FIG. 1, panels a and c). The monitoring for the tropomyosin-1 selections showed that only one round of selection was sufficient to enrich for tropomyosin-1 binders as polyclonal phage antibodies from the first round of selection already showed a signal in Western blotting experiments (FIG. 1, panel b). For PABPN1, the selection progress was monitored on HeLa cells, exemplifying that even endogenous protein present in a complex cell extract can be used (FIG. 1, panel d). The monitoring process can also reveal unsuccessful selections or the selection for (immunodominant) impurities. Therefore, immediate monitoring of the specificity of polyclonal phage antibodies can effectively prevent the time-consuming monoclonal antibody fragment characterization following selections.

### Polyclonal Phage Antibodies for Detection of Endogenous Antigen

As polyclonal phage could be applied successfully to monitor selection progress with Western blotted recombinant antigen we sought to investigate their performance for binding endogenous human protein. To this end, polyclonal phage antibodies were used directly to evaluate binding to their associated antigens in a HeLa cell extract (FIGS. 1d and 2). Endogenous tropomyosin-1, actin and PABPN1 could be detected in a HeLa cell extract with the polyclonal phage outputs from the second round selections (FIG. 1, panel d; FIG. 2, panels b and c). This demonstrates that the polyclonal phage antibodies contain a high proportion of phage-bound VHH that is suitable for Western blotting and able to bind the human protein. Polyclonal phage outputs from the selections against emerin did not give a specific signal (FIG. 2, panel a).

To determine the detection limits of polyclonal phage antibodies for endogenous tropomyosin-1 in muscle, serial 5-fold dilutions of a human muscle homogenate were blotted onto PVDF membrane in parallel with serial dilutions of the recombinant tropomyosin-1 in known concentration. The second round polyclonal phage antibodies from the selection against tropomyosin-1 were titrated and 10⁴ TU/ml phage was used to incubate the membrane. FIG. 3 shows that the signal intensity for tropomyosin-1 decreased with decreasing amounts of human muscle protein and demonstrates that polyclonal phage antibodies could be used for the detection of less than 50 ng of endogenous tropomyosin-1 in a human muscle homogenate.

### Screening for Monoclonal Antibody Fragments

To attain monoclonal sources of the selected antibody fragments, 96 randomly selected clones per antigen were screened for binding to their respective targets. Screenings were performed both by identification of individual antibody fragment binding capacities and by determination of genetic diversity. Culture supernatants from overnight inductions containing antibody fragments were tested in ELISA using directly coated recombinant antigen, thus resembling the second round of selection. In parallel, the genetic diversity of the selected clones was determined with single-colony PCR and restriction pattern analysis for the frequent cutting enzyme HinfI. For all four antigens 50-80% positive clones were identified (Table 11) while a high degree of genetic variability in the antigen-specific clones was observed for the emerin and actin selections. The cDNA of VHH that were positive in ELISA and showed different restriction patterns were sequenced revealing that 22, 4 and 1 different VHH were selected for emerin, actin and tropomyosin-1, respectively (Table 11).

Detection of Endogenous Proteins with Monoclonal Phage and Purified Antibody Fragments

Phage produced from individual clones that were identified by ELISA and fingerprint analyses were tested for their ability to detect Western blotted endogenous antigen. For all four antigens, monoclonal phage-VHH could be identified that specifically bound to the endogenous antigens in a HeLa cell extract on Western blot (FIG. 1, panel d; FIG. 2, panels a, b, c). It is noteworthy that for tropomyosin-1, actin and PABPN1 signal intensities for the monoclonal phage incubations were comparable to the signals obtained with the polyclonal phage antibodies. Since identical phage dilutions were used, this confirms the high proportion of antigen recognizing phage present in the polyclonal phage pool. For emerin, where polyclonal phage antibodies could not detect endogenous antigen, monoclonal phage clones could be identified that clearly and specifically bound emerin in the HeLa cell extract (FIG. 2, panel a).

Next, we investigated the performance of purified antibody fragments to detect the endogenous antigens in cell extracts and tissue homogenates by Western blotting. Soluble antibody fragments from the phage clones identified by the screenings were purified from periplasmic preparations of E. coli inductions. Yields up to 2 mg/l culture volume were obtained and purified antibody fragments were used to probe Western blotted HeLa cell extracts and tissue homogenates. For all four antigens the selected VHH could recognize the endogenous antigens in a HeLa cell extract (FIG. 4, panels a-c). The VHH anti-tropomyosin-1 and anti-actin successfully detected the endogenous antigens in muscle as well (FIG. 4, panel d).

### Immunofluorescence Microscopy

Immunofluorescence is another step in the screening process to increase the probability that from the originally selected VHHs or VHH carriers those or obtained that recognize the antigen in its disease related tissue or cell.

The applicability of our antibody fragments for immunofluorescence microscopy was investigated using primary fibroblast cultures. Application of the VHH for tropomyosin-1 and actin in fluorescent immunocytochemistry demonstrated that the anti-TPM1 VHH (G4) localized in thin filaments (FIG. 5, panel □) while the different anti-ACTA1 VHH (D7, B5, B8) marked different parts of the cytoskeleton, though, not always associated with the F-actin network of the fibroblasts' stress fibers (FIG. 5, panels q, u and y). With the anti-emerin and anti-PABPN1 VHH, the localization of emerin and PABPN1 was studied in normal primary fibroblasts and in primary fibroblasts derived from a patient with a homozygous nonsense Y259X mutation in the LMNA gene which causes complete absence of lamins A and C. PABPN1 was localized in nuclear speckles and the nucleoplasm in both control fibroblasts and patient cells (FIG. 5, panels i and m) as was shown previously for other pre-mRNA splicing factors [27][Vecerova J 2004, Mol. Biol. Cell 15, 4904-4910]. As lamins are crucial for the formation of the nuclear lamina, the nuclear integrity is lost in these cells and emerin is mislocalized into the endoplasmatic reticulum (ER) [25] [Muchir A. et al. 2003, Exp. Cell Res. 291, 352-362]. Staining of emerin with VHH EME7E and co-staining for lamins A and C showed that our selected antibody fragment recognizes emerin in the nuclear membrane in control cells (FIG. 5, panel a) while the expected mislocalization to the ER was observed in the patient cells (FIG. 5, panel e). Consistent with this staining pattern is the complete absence of signal for the anti-lamin polyclonal antibodies in the patient cells while showing normal immunoreactivity at the nuclear membrane of control fibroblasts (FIG. 5, panels g, o and c, k, respectively).

Finally, we investigated the applicability of the VHH for immunohistochemistry. In thin cryosections from muscle, the antigens were successfully detected with VHH specific for actin, tropomyosin-1 and PABPN1 (FIG. 6) and emerin (FIG. 7). The typical patterns for actin and tropomyosin were obtained when the respective VHH (anti-actin VHHA2 and anti-tropomyosin-1 VHHG4) were used in transverse and longitudinal sections, respectively (FIG. 6, panels a and d). For PABPN1 a nuclear labeling was obtained (FIG. 5, panel g). Emerin was specifically detected in the nuclear rim of muscle nuclei as seen in thin cross sections and its specificity and applicability as diagnostic marker was further confirmed by the absence of emerin immunoreactivity in the muscle of a EDMD patient (FIG. 7).

### Discussion

Current genomics- and proteomics-based high-throughput technologies create a great demand for functional characterization of proteins and their modifications. Conventional monoclonal antibody generation by the hybridoma technique is expensive and time-consuming and is unlikely to keep up with the current pace of gene identification techniques. The use of antibody phage display of nonimmune repertoires provides a cost-effective, flexible and fast alternative to generate large panels of antibody fragments [28] [Bradbury A R & Marks J D 2004, J. Immunol. Methods 290, 29-49]. However, the use of conventional antibody repertoires in combination with phage display suffers from important drawbacks related to the uncontrolled combination of VH and VL genes that renders the majority of combinations non-functional [c7] [Marks et al. 1992, Biotechnology 10, 779-783]. Unique sources of functional antigen binding domains that are encoded by single genes have become available by the combination of Camelid single-domain antibody fragments with phage display [c8, 9] Arbabi Gharhoudi M et al. 1997, FEBS Lett. 414, 521-526; v. d. Linden R et al. 2000, J. Immunol. Methods. 240, 185-195].

To provide a solution for the increasing demand for immunological reagents, we developed a fast, reliable and controllable protocol for the selection of antibody fragments from a nonimmune llama VHH library. We selected single-domain antibody fragments for four antigens that represent different subcellular structures and play a role in diverse muscle disorders. The isolated clones are a source of monoclonal antibody fragments amenable to engineering and can be applied to various immunological techniques.

Changes in the epitope accessibility or changes in the antigen itself, caused by its immobilization [29] [Smith A D & Wilson J E 1986, J. Immunol. Methods 94, 31-35], can lead to selection of antibody fragments that will not recognize the native antigen. By capturing the antigen by means of a tag sequence, the antigen is presented “in solution” and it is likely to appear with more epitopes accessible for phage binding in comparison with the use of antigen-specific antibodies or non-directional immobilization. Moreover, antigen immobilization by capturing acts as an affinity purification step and therefore decreases the probability to select for impurities that are occasionally present in antigen preparations. Immobilization of the antigen in both rounds of selection solely with the anti-T7 antibody resulted in enrichment for the capture agent, even when excess of irrelevant monoclonal antibody was used for competition. By comparative evaluation we have defined a specific order of antigen immobilization to be followed during selection with antigen capturing to occur in the first round and selection according to the biopanning protocol in the second round. While with this order diverse antibody fragments could be obtained that were capable to detect the endogenous antigen in different applications, the reverse order of antigen presentation failed to yield functional antibody fragments.

Additionally, our studies show that the antigen configuration is equally important to its presentation. Although full-length tropomyosin-1, actin, PABPN1 and the intranuclear domain of emerin were used for selections with the aim to select VHH against different parts or conformational domains of each antigen the diversity in the selection outputs was very different. While several antibody fragments were isolated for emerin and actin, it is likely the presence of an immunodominant region in tropomyosin-1 that resulted in the isolation of a single VHH clone as confirmed by monoclonal phage ELISA, DNA fingerprinting and subsequent sequence analysis of the positive clones. The marginal diversity in this case was expected during the evaluation of the selections since polyclonal phage from the first selection round could already immunoreact with the endogenous tropomyosin-1 of human muscle homogenates on phage Western blots (data not shown). Possibly, through epitope-masking [21, 22] [Ditzel H J, 1995, J. Immunol. 154, 893-906; Sanna P P et al. 1995, Proc. Natl. Acad. Sci. 92, 6439-6443]by using this predominant VHH as capture agent for new selections, different antibody fragments may be obtained for tropomyosin-1.

While emerin and PABPN1 are encoded by single genes, both actin and tropomyosin-1 belong to gene families, comprising 6 and 4 highly homologous members, respectively. After two rounds of selection monoclonal VHH for emerin and PABPN1 recognize a single protein of expected molecular weight. The selections for actin and tropomyosin-1 yielded VHH that could recognize multiple homologues or isoforms as evidenced by Western blotting (FIG. 4) or immunofluorescence microscopy (FIG. 5). Tropomyosins are ubiquitous proteins of 35 to 46 kDa associated with the actin filaments of myofibrils and stress fibers. In vertebrates, at least 4 known tropomyosin genes code for diverse isoforms that axe expressed in a tissue-specific manner and regulated by an alternative splicing mechanism Lees-Millar J P & Helfman D M 1991, Bioessays 13, 429-437]. Our Western blot analyses with polyclonal and monoclonal phage show that in human muscle homogenates as well as in HeLa cell extracts different isoforms are detected. This observation is in line with our assumption that the presence of a highly conserved immunodominant domain in tropomyosin-1 resulted in the selection of a single antibody fragment with high affinity for all tromomysin isoforms.

In the case of actin, since all actin isoforms have the same molecular weight of 45 kDa, it is not possible to discriminate them by Western blotting (see also FIGS. 2 and 4). However, the higher degree of VHH diversity observed after the selection process seems to be reflected in the different cytoskeletal substructures recognized by the individual antibody fragments in immunofluorescence microscopy experiments. Although we have not determined the exact conformational epitopes that mark the discrete components of the cytoskeleton by the various anti-actin VHH (FIG. 6), it is evident that the diverse antibody fragments exhibit different properties as immunoprobes.

While selection is very rapid and can be done for multiple antigens in parallel, screening for individual antibody clones that perform well in the intended application remains relatively labor intensive. As a solution to a more time-effective screening approach that can involve large numbers of antigens, and to prevent labor-intensive follow-up of unsuccessful selections, we developed a ‘real-time’ monitoring system for phage display selections. We demonstrated that in parallel with the selection procedure, the progress in terms of positive binders could be effectively evaluated by Western blotting of recombinant antigen and using total phage pools from each round of selection for antigen detection. These binder-monitoring experiments can also reveal the co-selection for immunodominant impurities that directly associate with the recombinant antigen.

Although polyclonal phage antibodies can be used efficiently to monitor the selection progress (FIG. 1), it is possible that the endogenous antigen cannot be detected, neither in a cell extract nor in a tissue homogenate, as was seen for the polyclonal phage antibodies from the selections for emerin. As the monoclonal phage effectively recognizes emerin in a HeLa cell extract (FIG. 2) we investigated if emerin could be detected with serial dilutions of polyclonal phage antibodies. With increasing phage concentration, the background increases to such an extent that it probably masks specific signals so that emerin could not be detected (data not shown). Indeed, for tropomyosin-1 (FIG. 3) and actin (data not shown), polyclonal phage antibodies specifically recognize the endogenous antigens in a human muscle homogenate. Therefore, for some antigens already after one or two rounds of selection with a nonimmune library, polyclonal phage antibodies can already be used to detect the endogenous antigen, which is in this sense comparable to a conventional polyclonal antiserum.

Using fibroblast cultures we have demonstrated the value of our VHH for target validation studies as we could confirm the mislocalisation of emerin in patient-derived fibroblast cultures carrying a nonsense Y259X mutation in the LMNA gene. Although the EMD gene is unaffected in these patients, as a secondary effect of the lamins A and C absence, emerin is dispersed throughout the encloplasmatic reticulum. We could also demonstrate the diagnostic applicability of the isolated antibody fragment by showing the loss of emerin in muscle cell nuclei of an EDMD patient as a consequence of a mutation in the EMD gene.

In conclusion, the synergy of phage display techniques and the optimized selection methods for heavy-chain antibody fragments from non-immune libraries holds great promise for future large-scale target validation in a cost-effective way. As this procedure does not require time-consuming immunization protocols, and parallel selections for many antigens is amenable to automation, large panels of antibody fragments can rapidly be obtained. Moreover, the flexibility in selection strategy, including the choice of epitope, the mode of selection (e.g. biopanning, capturing, or epitope-masking), renders these antibody fragments and their genetically modified derivatives useful tools for proteomics to correlate function and pathology to genomic alterations, both in biology and medicine.

### EXAMPLE 2

### Komt Niet Terug in PDF File

### Vind Ik Uitstekend

### Material and Methods

### Antibody Selections

The human cDNA sequence of PABPN1 was cloned into the prokaryotic expression vector pET28a (Novagen). Recombinant protein was produced in BL21(DE3)-RIL E. coli (Stratagene). The protein was purified by means of the attached His-tag using TALON (BD Biosciences). Two rounds of selection were performed with a large (5*10⁹) non-immune llama single-domain antibody fragment library (kindly provided by Unilever Research Vlaardingen, The Netherlands), using standard procedures.[15] [Verheesen P. et al 2003, Biochim. Biophys. Acta 1624, 21-28] Briefly, with differences as described: monoclonal antibody against the T7-tag (Novagen) (10 μg/ml in PBS (137 mM NaCl, 2.7mM KCl, 8 mM Na₂HPO₄, 1.5 mM KH₂PO₄)) was coated to maxisorp 96-well plates (Nunc). After blocking with 4% skimmed milk in PBS (4% MPBS) the purified T7-tagged. PABPN1 (10 μg/ml in 0.1% MPBS) was captured. 10¹¹ phages (in 2% MPBS, 1% bovine serum albumin (BSA), 10% normal mouse serum (NMS)) were added and incubated for 90 min. at room temperature (RT). After extensive washing with 0.05% Tween-20 in PBS (PBST) and PBS, bound phages were eluted with 100 mM Triethylamine (TEA) for 10 min. at RT. Phages were prepared for the second selection round as described. Bound phages were eluted with high pH. These phages were multiplied and used for a second selection round. This second selection round was performed similar to round 1 except that PABPN1 (10 μg/ml in PBS) was directly coated to maxisorp plates and 10⁹ phages were used. After the second selection round, single colonies were picked in 96-well plates and c-myc-tagged VHH (VHH-myc) were produced by overnight induction in 96-well plates. Culture supernatants containing VHH-myc were tested with ELISA for binding to directly coated PABPN1 (10 μg/ml in PBS). VBH-myc were detected with mouse anti-c-myc antibodies (kind gift from P. W. Hermans, Biotechnology Application Centre, The Netherlands) and anti-mouse peroxidase-conjugated antibodies (Jackson). Single-colony PCR was performed as described, PCR fragments were cut with HinfI (New England Biolabs) and analyzed on 2% agarose gels.

### Epitope Mapping

The following PABPN1 domains were PCR-amplified from the full-length PABPN1 cDNA and cloned in a derivate of GST-fusion vector pGEX-3× (Amersham): Oligomerization domain 264-306□ (OD₍₂₆₄₋₃₀₆₎); oligomerization domain 155-294(OD₍₁₅₅₋₂₉₄₎): amino acids 173-244 [Fan X et al. 2001, Hum. Mol. Genet. 10, 2341-2351]that contain most of the RNP-domain that stretches from amino acids 161-257[c16] [Tavanez J P. Et al. 2005, RNA 11, 752-762 (RNP₍₁₇₃₋₂₄₄₎), and amino acids 271-291 that contain a cluster of methylated arginines(AP₍₂₇₁₋₂₉₎). Deletion constructs DN10, DN49, DN92, DN113, an N-terminal protein fragment encoding amino acids 1-125, and point mutation constructs V126S, M129A, E131A, A133S, K135A, L136S, V143A (WB: 040315) were used for fine epitope mapping and were described before. □ [Kerwitz Y et al. 2003, EMBO J. 22, 3705-3714]] (kindly provided by Uwe Kuhn, Martin-Luther-University Halle, Germany) and supplied ready-to-use as purified proteins for Western blotting.

### Affinity Measurements

Association and dissociation constants (kₒₙ and k_{off}, respectively) for the binding of 3F5 to PABPN1 were measured using a Biacore3000 (Biacore) and covalently coupled recombinant PABPN1 and 3F5 to a CM5 sensor chip. Affinities were calculated using Biacore evaluation software.

### Cell Culture and Immunofluorescent Labeling

HeLa and COS-1 cells were cultured according to standard protocols. Cells were grown on coverslips for 24 h, washed with PBS and fixed with 4% formaldehyde in PBS for 15 min. at RT. Triton X-100 was added to a final concentration of 0.1% and cells were permeabilized for 15 min. at RT. Cells were blocked with 100 mM glycine in PBS and 1% BSA in PBS both for 15 min. at RT and incubated with 3F5 (1 μg/ml in 1% BSA/PBS) for 90 min. at RT. VHH were detected with anti-c-myc monoclonal antibody and Alexa Fluor 488-labeled anti-mouse antibody (Molecular Probes) in 1% BSA/PBS, each for 1 h. Cells were incubated with 0.2 μg/ml DAPI (Roche) together with the last antibody incubation to visualize nuclei. 6 μm cross-sections from a control human muscle, biopsy were air-dried for 30 min, fixed and labeled with 3F5 as described for cultured cells.

### Transfections and Quantification of Aggregates

mPABPN1-ala17 was cloned into an eukaryotic expression vector (pSG8)^{[19]} adjacent to the C-terminus of the vesicular stomatitis virus glycoprotein-tag (VSV-tag). The VSV-tag allows specific immunological detection of the transfected mutant protein. The cDNA encoding 3F5 was cloned into an eukaryotic expression vector (modified pSG8) in fusion with the SV40 T-antigen nuclear localization signal (NLS) and the green fluorescent protein (GFP).

As a model for PABPN1 aggregate formation, COS-1 and HeLa cells were transfected with plasmid encoding VSV-tagged mutant PABPN1 with 17 alanines (mPABPN1-ala17) using FuGENE 6 (Roche, Indianapolis, USA). Cells were fixed and permeabilized 24 h and 48 h post-transfection as above. The transfected. PABPN1 was detected with mouse anti-VSV antibody (clone P5D4, Roche), followed by incubation with anti-mouse Cy3-conjugated goat antibody (Jackson, West Grove, USA). Cell nuclei were stained with DAPI (Roche, Mannheim, Germany).

Intrabody constructs with VHH in fusion with the SV40 T-antigen nuclear localization signal (NLS) and green fluorescent protein (GFP) were co-transfected and serially transfected in 0.5:1, 1:1, 2:1 and 4:1 intrabody:mPABPN1-ala17 ratios. mPABPN1-ala17 was visualized with anti-VSV antibody as described above and the intrabody was readily visible by virtue of the fusion with GFP. An unrelated intrabody and NLS-GFP were transfected together with mPABPN1-ala17 in 1:1 ratio as controls. For the serial transfections, 3F5, control intrabody and NLS-GFP alone were serially transfected in cells that already expressed mPABPN1-ala17 for 24 h. 48 h after this second transfection, mPABPN1-ala17 was visualized as described before. Three independent experiments were performed for assaying aggregate prevention and gene dosage effects. From the co-transfection and serial transfection experiments at least 200 and 100 transfected cell nuclei were scored for the presence of intranuclear aggregation, respectively.

With the SPSS package the dose dependence could be described by loglogistic regression according to the formula b1+ln(1+b2*exp(b3*[ag]) in which b1, b2 and b3 are the parameters to be estimated and [ag] the ratio aggregated/non aggregated cells in co-transfection.

### Cell Proliferation Assay

Possible toxic effects of the transient expression of our mPABPN1 and intrabody constructs were investigated with a MTT (3-[4,5-dimethylthiazol-2-yl]-2,5-diphenyl-tetrazolium bromide) (Sigma-Aldrich) cell proliferation assay that discriminates between dead and living cells based on their metabolic activity. MTT was dissolved in culture medium in 1 mg/ml concentration

### Western Blotting

Cytosolic and nuclear fractions of HeLa cells were prepared as described,[20] loaded on 12% SDS-polyacrylamide (SDS-PAGE) gels and transferred to PVDF Western blotting membranes (Roche). Membranes were incubated with 3F5 (1 μg/ml in 2% MPBS) overnight at 4° C., followed by incubation with anti-c-myc and anti-mouse peroxidase-conjugated antibodies. Co-transfected cells were trypsinized and lysed in Laemni-buffer. Lysates were loaded on 12% SDS-PAGE gels, transferred to PVDF Western blotting membranes and incubated with anti-VSV, anti-GFP (Roche) and anti-actin (ICN) antibodies.

### Results

### Antibody Fragment Selections

To isolate antibody fragments against PABPN1 two phage selection rounds were successively employed against E. coli-produced and affinity purified full-length human PABPN1. Previously, we demonstrated that a combination of capturing the antigen in the first round (by its T7 tag) followed by direct immobilization in the second round, yields the most divers set of binders (Verheesen, P. et al, in preparation). To ensure that different epitopes were recognized by the selected antibody fragments we performed epitope-masking selections in which antibody fragments obtained in earlier selection rounds were used to capture PABPN1. The enriched sub-libraries were screened for monoclonal antibody fragments with specificity for PABPN1. From the different selections a total of 6 different antibody fragments were identified with affinities for PABPN1 between 5 and 57 nM (data not shown).

### Epitope Mapping

Three selected antibody fragments were further characterized by epitope mapping with Western blotting using a panel of truncated recombinant PABPN1 proteins. All mapped to epitopes within the aminoterminal 155 amino-acids. One of the VHH, coded 3F5, obviously showed the strongest signals on Western blot. As this antibody fragment also exhibited the highest affinity (5 nM) for both the recombinant produced PABPN1 and native PABPN1 we continued with this antibody fragment for a more detailed epitope mapping. From these epitope mappings (FIG. 8) we conclude that the epitope for 3F5 is situated between amino acids 113 and 133, which overlaps with most of the part of PABPN1 that was predicted to form an amphiphatic □-helical or coiled-coil domain (amino acids L119-Q147) Kerwitz Y et al. 2003, EMBO J. 22, 3705-3714] Using a series of different point mutants of PABPN1, we could demonstrate that residues at position 126, 129 and 131 are essential for binding of the antibody fragment

### Detection of Endogenous PABPN1

3F5 was subsequently used for Western blotting with cytosolic and nuclear fractions from HeLa cells. A single band with an expected molecular weight of 50 kDa was specifically detected in the nuclear protein fraction. Next, in two independent cell lines, HeLa and COS-1 cells, we observed an expected predominant nuclear staining with denser fluorescent signal in a speckle-like pattern by immunofluorescence microscopy Krause et al. 1994, Exp. Cell. Res. 214, 75-82]. In addition to cultured cells, cryosections of control human muscle were stained with 3F5 as well. Nuclear localization with accumulation in a speckle-like pattern was observed, indicating successful detection of PABPN1 in muscle.

### Cell Model for PABPN1 Aggregation

To discriminate between endogenous and over-expressed mutant PABPN1 we transfected mPABPN1-ala17 in fusion with the vesicular stomatitis virus glycoprotein (VSV) tag in COS-1 and HeLa cells. Intranuclear aggregation of PABPN1 was observed. Incubation of these cells with fluorescent oligo(dT), anti-HSP70 and anti-ubiquitin antibodies showed that poly(A)-RNA, HSP70 and ubiquitin were present in the aggregates (data not shown), as has been reported previously [4, 9].[Calado A et al. 2000, Hum. Mol. Genet. 9, 2321-2328; Abu-Baker A 2003, Hum Mol. Genet. 12, 2609-2623]

We questioned whether transfected mPABPN1-ala17 was localized differently compared to endogenous PABPN1. To this end, HeLa cells were transfected with mPABPN1-ala17 and the transfected mPABPN1-ala17 was visualized by incubation with anti-VSV antibodies while total PABPN1 was visualized with 3F5. No differences in localization of both signals were observed [FIG. 11].

### Prevention of Aggregation by Intrabody 3F5

The consequences of intracellular expression of 3F5 were investigated in cellular PABPN1 aggregation models. Intrabody 3F5 was transfected in fusion with a nuclear localization signal (NLS) and green fluorescent protein (GFP) (3F5-NLS-GFP) in COS-1 and HeLa cells. The GFP signal was exclusively observed in the nucleus indicating both a successful expression of intrabody and its targeting to the nucleus.

Subsequently, 3F5-NLS-GFP was co-transfected with mPABPN1-ala17 in different ratios. In a dose-dependent manner in which mPABPN1-ala17 was kept constant and increasing concentrations of 3F5-NLS-GFP, the intrabody could completely prevent aggregation The expression levels of mutant PABPN1 and intrabody 3F5 or NLS-GFP control were analyzed by Western blotting. This showed that expression of the intrabody did not affect the expression levels of its antigen mPABPN1-ala17 [FIG. 12].

To explore eventual cytotoxic effects of the intracellular expression of 3F5, cells were single transfected with the intrabody construct and analyzed at different time points by Western blotting. A gradual increase in intrabody expression was observed in time without a detectable effect on endogenous PABPN1 levels. Cells that were single transfected with the intrabody construct were also microscopically investigated. PABPN1 was labelled with one of the selected antibody fragments that recognize a distinct epitope from the binding place of 3F5. The PABPN1 localization in intrabody expressing cells was indistinguishable from its localization in non-transfected cells. With a cell proliferation assay, the metabolic activity in intrabody-transfected cells was compared to non-transfected cells. No difference in metabolic activity was observed between these cells. Therefore, intracellular expression of 3F5 did not cause any detectable cytotoxic effects based on analysis of endogenous antigen levels and localization, and cell viability.

### Clearing of Existing Aggregates

To investigate whether pre-existing aggregates can be cleared with our intrabody we performed serial transfections with mPABPN1-ala17 and intrabody 3F5 in COS-1 and HeLa cells. Twenty-four hours after transfection, 38(±4) % of HeLa cells and 33(±8) % of COS-1 cells showed intranuclear aggregates. Intrabody 3F5, control intrabody or NLS-GFP control were serially transfected 24 hrs after transfection of mPABPN1-ala17. An increase in the percentage of cells showing aggregation in time was observed for the control intrabody and NLS-GFP control (140(±5) % and 116(±5) % respectively in HeLa cells). Similar increases in aggregate formation were observed in COS-1 cells transfected with either control intrabody or NLS-GFP control (126(±16) % and 135(±14) %, respectively). In contrast, a significant decrease in the number of cells with intranuclear aggregates was observed for the serial transfections with 3F5. In a 1:1 ratio of intrabody:mPABPN1-ala17 a reduction to 70(±4) % (p<0.05) was observed for HeLa cells while a reduction to 89(±10) % was observed in COS-1 cells [FIG. 14]

### Discussion

By a combination of antigen capturing, panning and epitope masking, we have selected various antibody fragments against PABPN1. Among these is an antibody fragment that may have potential in the treatment of OPMD as we demonstrate that it can reduce aggregate formation or clear already existing aggregates in a cell model for OPMD.

To date, the specific development of muscle defects in OPMD remains unclear. Aggregates of mutant PABPN1 are present in post-mitotic long living myonuclei of OPMD patients [4-6][Calado A et al. 2000, Hum Mol. Genet. 9, 2321-2328; Uyama E et al. 2000, Muscle Nerve 23, 1549-1554; Becher M W et al. 2000 Ann. Neurol. 48, 812-815], which may indicate a relationship between the differentiation state of the cell and the appearance of detectable inclusions [1]Brais B, 2003, Cytogenet. Genome Res. 100, 252-260]. It was shown that mutant PABPN1 aggregates contain high concentrations of poly(A)-RNA and it was suggested that poly(A)-RNA entrapment in aggregates may play a role in OPMD pathogenesis [4][Calado A et al. 2000, Hum Mol. Genet. 9, 2321-2328]. The muscle-specific phenotype may be further explained by sequestration of ski-interacting protein (SKIP) in the aggregates, as it is known that PABPN1 and SKIP synergistically activate MyoD [22][Kim Y J et al. 2001, Hum Mol. Genet. 10, 1129-1139]. Although the exact pathological mechanism underlying OPMD is only partly understood, cellular and animal models studies of OPMD are consistent with the view that the aggregation process, and more specifically early oligomeric mutant proteins, are toxic Brais B, 2003, Cytogenet. Genome Res. 100, 252-260; Calado A et al. 2000, Hum MeI. Genet. 9, 2321-2328 Abu-Baker A 2003, Hum Mol. Genet. 12, 2609-2623]].

Currently, over-expression of mPABPN1 in COS-1 and HeLa cells are the only cellular reporter systems that have been demonstrated to show mPABPN1-aggregation that leads to cell death [Bao Y P 2004, J. Med. Genet. 41, 47-51]. Inhibition of aggregate formation with chaperones, doxycycline and Congo red has been described Bao Y P 2004, J. Med. Genet. 41, 47-51]. However, these chaperones and chemicals are not specific for PABPN1 aggregates but rather recognize a large number of misfolded or aggregated proteins and may thus have undesired side-effects. In contrast, antibodies that specifically bind their target can be used for specific intervention. We have therefore selected a PABPN1-specific monoclonal antibody fragment for which we show that we are able to prevent aggregate formation by mutant PABPN1 in a dose-dependent manner. Intracellular expression of this antibody fragment did not yield any detectable detrimental side effects as assayed by normal antigen levels, localization or cell viability. The observation that endogenous and transient PABPN1 protein levels in transfected cells are normal, indicates that reduction of aggregate formation by the intrabody is a direct effect of the intrabody on the structure and not the level of the mutant protein. This concerns a very specific interaction, as other VHH against distinct epitopes on PABPN1 were not able to prevent aggregate formation (data not shown). Potential oligomerization domains have been identified in PABPN1 that were shown to play a role in aggregation [16] D. Binding of 3F5 to these or yet unidentified regions may prevent aggregation by shielding off these regions for other interactions similar as was shown with deletion constructs [16][Korwitz Y et al 2003, EMBO J. 22, 3705-3714].

In serial transfections of mPABPN1 and 3F5 we showed that the intrabody cannot only prevent aggregation, but can also clear already existing aggregates. Three independent transfection experiments in two different cell lines showed a significant reduction of aggregate-containing cells when a surplus of 3F5 was expressed in cells in which aggregates were already formed (p<0.05).

In conclusion, here we have shown that single-domain antibody fragments from Camelidae can function as intrabodies and highly selective block or revert pathological processes. Further studies will aim for efficient delivery of the single domain antibody fragments in cells of affected OPMD tissue to evaluate whether also in affected tissue, 3F5 will have preventive and curative properties. An intriguing question will be whether prevention and clearing of aggregates will result in the restoration of homeostasis of affected muscle.

### EXAMPLE 3

Capturing/Panning Selections

Single-domain antibody fragments were selected against PABPN1 in two rounds of selection. Full-length PABPN1 was captured in the first round of selection by means of its T7-tag (Novagen) with monoclonal antibodies. The second selection round was performed with direct coating of the full-length PABPN1. This combination and specific order of antigen immobilization was shown to yield the most diverse set of antibody fragments (Verheesen, P., Roussis, A., et al., in preparation). The selections were monitored with polyclonal phage on endogenous PABPN1 from HeLa cell lysate (Verheesen, P., Roussis, A., et al., in preparation). Specific enrichment for PABPN1 was observed (Figure in “Fast, reliable and controllable selection of phage display-derived antibody fragments from a Camelid nonimmune library”, Verheesen, P., Roussis, A., et al., in preparation). The selected antibody fragments were assessed for genetic diversity by fingerprint analyses. Binding to the selection antigen, E. coli-produced full-length PABPN1, was analyzed with ELISA. Genetically different clones that bound PABPN1 were sequenced [FIG. 9 gives a dendrogram based on amino acid sequence comparison of the selected and screened VHHa that recognized PABPN1]. Finally three different antibody fragments were selected for further in situ studies.

### Selections by Epitope-Masking

Previously selected antibody fragment 3F5 (see above) was used to capture native PABPN1 that was purified from bovine calf thymus (kindly provided by Dr Antje Ostareck-Lederer, Martin-Luther-University Halle-Wittenberg, Halle, Germany). Capturing with antigen specific antibody fragments blocks off antigenic sites and favors selection against other epitopes on the same antigen. This was shown to be of particular interest when a library is biased (I have to search for appropriate references when needed: Sanna 1995?, Ditzel 1995?), e.g. when an immune response is raised against other epitopes than aimed for in the selection process. Obviously, this is of less importance when a naïve library is used. Nevertheless, the presence of immuno-dominant epitopes in an antigen will influence antibody selection under all circumstances. By blocking off immuno-dominant epitopes, selection against less “immunogenic” epitopes may be favored. Consequently, this may result in selection of lower-affinity antibody fragments. The second selection round was performed with direct coating of the antigen {FIG. 10}

These antibody fragment selections were evaluated differently compared to the capturing/panning selections (above). An increase in proportion of phage-VHH eluted from the antigen in successive rounds of selection pointed towards successful selections. 288 clones were tested for genetic diversity with fingerprint analyses. 36 clones were sequenced based on the fingerprint analyses. The 20 different VHH on the protein level were produced, purified and tested in immuno-cytochemistry (Figure in General Introduction, thesis, Verheesen, P.□/The sequences of these VHH and the sequences of the VHH in were used for categorization [FIG. 9]. The screening step by immuno-cytochemistry is of particular interest as we aimed at isolating antibody fragments that bind PABPN1 when it is in complex with other proteins in the cell. When used as an intrabody, these VHH supposedly cause less adverse side effects. Again, three unique VHH were identified.

TABLE 12
VHH against PABPN1 selected by epitope-masking against native
PABPN1.

| | | |
|---|---|---|
| 08 | BIAcore affinity measurements: | 57 nM |
| 18 | BIAcore affinity measurements: | 7 nM |
| 29 | BIAcore affinity measurements: | 11 nM |

In an ELISA, the selection setup was mimicked. 3F5 was coated, native PABPN1 was captured, and 3A9, 3E9, 08, 18, 29 binding was investigated. For VHH 29, binding to a complementary epitope was confirmed. This antibody fragment is also very suitable for immunocytochemistry. The prevention of PABPN1 aggregation with the different VHH was tested in the cell model for OPMD. Interestingly, antibody fragment 29 is incapable to prevent aggregation. The antibody fragments from the capturing/panning selections, probably binding the same epitope, are capable to prevent aggregation. Please note that PABPN1 naturally oligomerizes. Maybe, this also happened during the selection process. Nevertheless, antibody fragments, e.g. 29, were selected that bind an other epitope than 3F5.

### EXAMPLE 4

### In Vivo Screening on Prevention of Aggregation

a. Cell Culture and Immunofluorescent Labeling

HeLa and COS-1 cells were cultured according to standard protocols. Cells were grown on coverslips for 24 h, washed with PBS and fixed with 4% formaldehyde in PBS for 15 min. at RT. Triton X-100 was added to a final concentration of 0.1% and cells were permeabilized for 15 min. at RT. Cells were blocked with 100 mM glycine in PBS and 1% BSA in PBS both for 15 min. at RT and incubated with 3F5 (1 μg/ml in 1% BSA/PBS) for 90 min. at RT. VHH were detected with anti-c-myc monoclonal antibody and Alexa Fluor 488-labeled anti-mouse antibody (Molecular Probes) in 1% BSA/PBS, each for 1 h. Cells were incubated with 0.2 μg/ml DAPI (Roche) together with the last antibody incubation to visualize nuclei. 6 μm cross-sections from a control human muscle biopsy were air-dried for 30 min., fixed and labeled with 3F5 as described for cultured cells.

b. Screening of Selected VHH Fragments on Prevention of Aggregation in Vivo

mPABPN1-ala17 was cloned into an eukaryotic expression vector (pSG8) adjacent to the C-terminus of the vesicular stomatitis virus glycoprotein-tag (VSV-tag). The VSV-tag allows specific immunological detection of the transfected mutant protein. The cDNA encoding the a selection of the VHH fragments given in, notably clones 08, 18, 29, 3A9, 3E9 and 3F5 were cloned into an eukaryotic expression vector (modified pSG8) in fusion with the SV40 T-antigen nuclear localization signal (NLS) and the green fluorescent protein (GFP). [FIG. 10]

As a model for PABPN1 aggregate formation, COS-1 and HeLa cells were transfected with plasmid encoding VSV-tagged mutant PABPN1 with 17 alanines (mPABPN1-ala17) using FuGENE 6 (Roche, Indianapolis, USA). Cells were fixed and permeabilized 24 h and 48 h post-transfection as above. The transfected. PABPN1 was detected with mouse anti-VSV antibody (clone P5D4, Roche), followed by incubation with anti-mouse Cy3-conjugated goat antibody (Jackson, West Grove, USA). Cell nuclei were stained with DAPI (Roche, Mannheim, Germany).

Intrabody constructs with VHH fragments 08, 18, 29, 3A9, 3E9 and 3F5 in fusion with the SV40 T-antigen nuclear localization signal (NLS) and green fluorescent protein (GFP) were co-transfected and serially transfected in 0.5:1, 1:1, 2:1 and 4:1 intrabody:mPABPN1-ala17 ratios. mPABPN1-ala17 was visualized with anti-VSV antibody as described above and the intrabody was readily visible by virtue of the fusion with GFP. An unrelated intrabody and NLS-GFP were transfected together with mPABPN1-ala17 in 1:1 ratio as controls. Three independent transfections with VHHs fragments were performed and the results of this in vivo screening are given in below

From the results depicted in FIGS. 9 and 10 it is clear that neither from biochemical characterization, nor from bio-informatic methods like amino acid homology searches the functioning of VHH fragments in vivo can be predicted. Whereas fragments 29 and 3A9 and 3F5 are quite homologous in amino acid sequence (see table 2), 3A9 and 3F5 show prevention of aggregate formation whether fragment 29 do not provide any prevention against aggregation. 3F5 proved to be the best candidate and is therefore used in further studies.

Concluding the in vivo screening is an essential step in the selection of VHH fragments with the desired properties.

c. Cell Proliferation Assay

Possible toxic effects of the transient expression of our mPABPN1 and intrabody constructs were investigated with a MTT (3-[4,5-dimethylthiazol-2-yl]-2,5-diphenyl-tetrazolium bromide) (Sigma-Aldrich) cell proliferation assay that discriminates between dead and living cells based on their metabolic activity. MTT was dissolved in culture medium in 1 mg/ml concentration

d. Western Blotting

Cytosolic and nuclear fractions of HeLa cells were prepared as described,[20] loaded on 12% SDS-polyacrylamide (SDS-PAGE) gels and transferred to PVDF Western blotting membranes (Roche). Membranes were incubated with 3F5 (1 μg/ml in 2% MPBS) overnight at 4° C., followed by incubation with anti-c-myc and anti-mouse peroxidase-conjugated antibodies. Co-transfected cells were trypsinized and lysed in Laemni-buffer. Lysates were loaded on 12% SDS-PAGE gels, transferred to PVDF Western blotting membranes and incubated with anti-VSV, anti-GFP (Roche) and anti-actin (ICN) antibodies.

e. Cellular Localization of Transfected VHH Fragments

The consequences of intracellular expression of 3F5 were investigated in cellular PABPN1 aggregation models. Intrabody 3F5 was transfected in fusion with a nuclear localization signal (NLS) and green fluorescent protein (GFP) (3F5-NLS-GFP) in COS-1 and HeLa cells. The GFP signal was exclusively observed in the nucleus indicating both a successful expression of intrabody and its targeting to the nucleus.

f. Determination of Dose Effects

Subsequently, 3F5-NLS-GFP was co-transfected with mPABPN1-ala17 in different ratios. In a dose-dependent manner in which mPABPN1-ala17 was kept constant and increasing concentrations of 8F5-NLS-GFP, the intrabody could completely prevent aggregation. The expression levels of mutant PABPN1 and intrabody 3F5 or NLS-GFP control were analyzed by Western blotting. This showed that expression of the intrabody did not affect the expression levels of its antigen mPABPN1-ala17.

To explore eventual cytotoxic effects of the intracellular expression of 3F5, cells were single transfected with the intrabody construct and analyzed at different time points by Western blotting. A gradual increase in intrabody expression was observed in time without a detectable effect on endogenous PABPN1 levels. Cells that were single transfected with the intrabody construct were also microscopically investigated. PABPN1 was labelled with one of the selected antibody fragments that recognizes a distinct epitope from the binding place of 3F5. The PABPN1 localization in intrabody expressing cells was indistinguishable from its localization in non-transfected cells. With a cell proliferation assay, the metabolic activity in intrabody-transfected cells was compared to non-transfected cells. No difference in metabolic activity was observed between these cells. Therefore, intracellular expression of 3F5 did not cause any detectable cytotoxic effects based on analysis of endogenous antigen levels and localization, and cell viability.

### EXAMPLE 5

### Clearing of Existing Aggregates

To investigate whether pre-existing aggregates can be cleared with our intrabody we performed serial transfections with mPABPN1-ala17 and intrabody 3F5 in COS-1 and HeLa cells. Twenty-four hours after transfection, 38(±4) % of HeLa cells and 33(±8) % of COS-1 cells showed intranuclear aggregates. Intrabody 3F5, control intrabody or NLS-GFP control were serially transfected 24 hrs after transfection of mPABPN1-ala17. An increase in the percentage of cells showing aggregation in time was observed for the control intrabody and NLS-GFP control (140(±5) % and 116(±5) % respectively in HeLa cells). Similar increases in aggregate formation were observed in COS-1 cells transfected with either control intrabody or NLS-GFP control (126(±16) % and 135(±14) %, respectively). In contrast, a significant decrease in the number of cells with intranuclear aggregates was observed for the serial transfections with 3F5. In a 1:1 ratio of intrabody:mPABPN1-ala17 a reduction to 70(±4) % (p<0.05) was observed for HeLa cells while a reduction to 89(±10) % was observed in COS-1 cells.

This demonstrates clearly that existing aggregates can be dissolved by VHH fragments.

Further Evaluation of Aggregate Clearing in Time

The potency to clear or dissolve already present PABPN1 aggregates was studied with serial expression of mutant PABPN1 (PABPN1-ala17) and intrabody 3F5. The illustrates expected phenomena to occur in such experiments. Aggregates are being formed in time in singly transfected cells with mPABPN1 (solid line). Immediate co-expression of intrabody 3F5 with mPABPN1 results in decreased aggregation (dotted line with big dots). When expressing the intrabody after expressing mPABPN1, an inhibitory effect is expected (dotted line with small dots). When even less aggregation would be observed, this could indicate solubilization of already present aggregates (gray area).

Indeed, a reduction of aggregation was observed by serial expression of intrabody 3F5. The main points of discussion in the experiments below are:
Only cells that show visible expression of mPABPN1 and intrabody were scored for the presence of aggregates
The mPABPN1 expression is always on. So, when the intrabody expression starts, mPABPN1 expression is already ongoing for 24 h and continues. The intrabody has to catch up with this expression.
There will be cells with high mPABPN1 and low intrabody levels, and otherwise.

The shows the relative aggregation that is observed in another serial transfection experiment. The aggregation at the moment of intrabody or control transfections was set to 100%. This figure shows the effect of different amount of the PABPN1 specific intrabody 24 h after serial transfection. Note this figure refers 24 h whereas refers to the effect of intrabody serial expression 48 h after serial transfection.

### EXAMPLE 6

### Selection and In Vitro and In Situ Screening of VHH that Prevent and/or Dissolve Amyloid-b Aggregates

A specific aspect of the present invention is that screenings are preformed to select VHH that prevent or even dissolve aggregates both in vivo and in vitro. Example 4 gives the in vivo screening on an mimic of an aggregate related to the neuromuscular disease OPMD. This example deals with the selection and in vitro and in situ screening of VHH that recognize and prevent aggregation of amyloid-β and the dissolvement of this aggregate.

Using the same methods as described in example 1, an number of VHH domains were selected that recognized amyloid b-42, a protein fragment present in aggregates in the brain that are related to Alzheimer's disease. The selected VHH-carriers were characterized and the DNA encoding the VHH domains was analyzed with restriction enzymes and their nucleotide sequence has been determined, similar to the procedures described in example 1.

The results of the nucleotide sequence analysis is given in table 5.1

It should be stressed that the VHH coding sequence started in all cases with the amino acid sequence QVQ or AVQ or QVK. This may be real but it may also be that this is due to the primer used for the cloning of the DNA encoding the domain.

Another important factor is that the first CDR start—according to Kabat numbering directly after the sequence: C(22)-A(T/K)-A-S-G-R(S)-T-F(R)-S(T/P) CDR 2 starts directly after the well conserved sequence K-E(Q)-R-E-F(L) [which sequence contain one of the Hallmarks of VHH's as described earlier in this patent application] followed by V(I/L)-A.

CDR 3 Starts Directly after C(92)-A(Y)-S/A(T)

Based on the DNA sequencing 6 individual VHH domains have been screened on biochemical properties, cross reactivity to Amyloid-β-40 and Western blots and finally and most importantly in on an in situ immunohistochemical methods. For the latter Human brain cryosections of patients with Dutch type of hereditary cerebral hemorrhage with amyloidosis)HCHWAD, MIM 609065) were used. All 6 individual domains showed staining compatible with vascular amyloid deposition, indicating recognition of Amyloid-b-42 in its natural context by the VHH domains.

Table 5-2 summarizes some of the results obtained.

From the domains listed in table 5.2, 7B and 8B showed the strongest immunohistoreactivity. Indications are obtained that these domains may prevent and/or dissolve these depositions.

1 10 20 30 40
EVQ LQA SGG G LVQ AGG SLR L SCA ASG FKI T HYT MGW FRQ A
41 50 a 60 70 80
PGK ERE FVS R ITW GGD NTF Y SNS VKG RFT I SRD NAK NTV YL
81 a bc 90 100 abc d e 110 120
QMN SLK PED T ADY YCA AGS T STA TPL RVD Y WGK GTQ VTV S S
1 10 20 30 40
EVQ LQA SGG G LVQ AGG SLR L SCS ASV RTF S IYT MGW FRQ A
41 50 a 60 70 80
PGK ERE FVA G INR SGD VTK Y ADF VKG RFS I SRD HAK NMV YL
81 90 100 a b c d ef g h 113 120
QMN SLK PED T ALY YCA ATW A YDT VGA LTS G YNF WGQ GTQ V TVS S

Preferred amino acid sequences of VHH domains which can translocate via Blood Brain Barrier to the brain.

The underlined amino acids represent the CDRs. Substitution of one or two amino acids of the CDR's by the amino acid indicated in my previous table may have also the property to pass the Blood Brain Barrier as well.

Substitutions of the Frame work residues to improve the functional and biophysical properties of the VHH domains are desired. However the substitutions should be restricted to those mentioned to amino acid at any position as given in the Entropy Variability table.

### EXAMPLE 7

### Beta-Amyloid

To obtain beta amyloid specific (Aβ1-40 and Aβ1-42) VHHs, selections were performed against Aβ1-40 or Aβ1-42 from a non-immune llama-derived heavy chain phage display library. This yielded 5 fragments (3A; 8B; 1B; 11G and 4D) (FIG. 16) that were tested for their reactivity for Aβ1-40 and Aβ1-42 by surface plasmon resonance (SPR) analysis and immunohistochemistry on brain cryosections of controls, patients with Alzheimers disease (AD), Down syndrome (DS) or vascular dementia (HCHWA-D). The SPR analysis shows a specific binding of the VHHs for their antigen Aβ 1-40 and Aβ 1-42 (FIG. 17).

Immunohistochemical analysis provides evidence for specific reactivity for beta amyloid deposition, most notably the angiopathy, but also, to some extend reactivity for parenchymal deposits (FIGS. 18 and 19).

In order to increase the avidity, VHH can be cloned in tandem to yield so-called biheads. The competence of the homologous 8B-8B bihead to detect immobilized amyloid Aβ 1-42 in vitro and amyloid deposits in the frontal cortex in vivo was subsequently tested by SPR analysis and on cryosections of affected brain by immunohistochemistry.

The avidity of the binding of the homologous 8B-8B bihead is shown in FIG. 20. The immunostaining evidently shows that the bihead contains increased capacity to detect these amyloid deposits in the brain (FIG. 21).

To aim at transmigration of the antibody fragments across the blood-brain barrier (BBB), two bifunctional biheads were created in which the anti-amyloid property of 3A or 8B was fused to the capacity of VHH FC5 [1] to cross the BBB. The immunoreactivity towards Aβ1-42 of the heterologous FC5-3A and FC5-8B biheads is comparable with the previous SPR analysis (data not shown). Furthermore, the capacity of the heterologous FC5-3A and FC5-8B biheads to detect amyloid deposits in the brain was subsequently tested on cryosections of affected brain by immunohistochemistry. These results clearly show that fusing 3A or 8B to FC5 does not interfere with amyloid recognition (FIG. 22).

### REFERENCE LIST TO EXAMPLE 7

1. Muruganandam A, Tanha J, Narang S, Stanimirovic D: Selection of phage-displayed llama single-domain antibodies that transmigrate across human blood-brain barrier endothelium. FASEB J 2002, 16: 240-242.

### EXAMPLE 8

### NMR Results

NMR has been selected as the technique of choice to investigate the characteristics at atomic level of the complex between the selected VHH's and β-amyloid (βA).

At the moment five different VHH's have been selected (two against βA 1-42 and three against βA 1-40. A first screening has been considered necessary in order to establish which one of the selected VHH's could be the most promising for the final goal of the project specifically regarding the affinity with its antigen.

Two different VHH's have been successfully labeled with ¹⁵N, a necessary step for the NMR study.

Chemical shift perturbation is a NMR technique which allows identifying the residues on the VHH surface affected during the complex formation with the βA.

Since chemical shift are very sensitive to variations in the local electronic environment, small changes in. ¹HN and ¹⁵N shift of the ¹⁵N labeled protein (VHH) on the [¹H-¹⁵N] HSQC spectra could be observed upon titration with no labeled βA. Shift are shown only by residues involved in the binding interface and they can be used to map the interface of the complex.

Via the NMR titration experiment information can be obtained concerning the strength of the protein-peptide interaction. In fact, according to the strength of the complex, different phenomena can be observed on HSQC spectra during the NMR titration: for complex involved in a weak binding, corresponding to a fast exchange regime in the NMR time scale, the (VHH) amide signal of the residues involved in the complex will shift upon interaction with the peptide and each position will be determined by the average position of the bound and the free form. In case of tight binding, two different signals will be present in the HSQC spectra for the free and for the bound form.

In the experiment here presented, 0.1 mM of ¹⁵N VHH-8B and VHH-3A have been titrated separately with a stock solution of 1 mM βA 1-42 (peptide against which the have been selected) at 303K in 20 mM phosphate buffer pH 7.

Superimposing VHH-8B HSQC spectra (FIG. 23) in the free form (black) and in the bound form with 0.8 (blue) and >1 equivalent (red) of βA 1-42, it is possible to locate the amide groups affected by the binding (depicted in FIG. 24 with a red circle). The observed changes in the chemical shifts indicate that free and bound VHH-8B conformations are in rapid exchange. Since no HSQC assignment of VHH-8B is yet available, it is not possible to identify the area affected upon βA 1-42 binding. However, from a comparison with a published work on a different VHH in which they claim that the most significant shift are in the CDRS region (Ferrat G. et all. 2002), it is possible to expect that also in this case the area involved in the biding could be the same.

In conclusion the binding between VHH-8B and βA 1-42 seems to be specific although weak.

On the other hand, different results were shown during a similar titration of VHH-3a with βA 1-42. During the titration (FIG. 24), few peaks were disappearing and new peaks appearing (peaks are depicted with red circle in FIG. 24), as in a slow exchange regime. Furthermore, the increased dispersion of the VHH peaks upon addition of the βA 1-42 seems to indicate a contribution of the βA to the VHH folding. In this case the binding between VHH 3A and βA 1-42 seems to be tight although since the small concentration of the sample a new experiment is required before any distinct conclusion. Interestingly, the two VHHs 8B and 3A, with only a different residues in position 15, 16 and 18 (FIG. 25), faraway from the CDR's, seem to have two different binding modalities with βA.

### EXAMPLE 9

### Construction of Bi-Functional VHHs for Non-Invasive Imaging and Dissolvement of Amyloid Fibrils in the Brain

Imaging of brain disorders is for patients and economically of large importance. However non-invasive imaging is only possible with labels that are linked to molecules that pass the Blood-Brain-Barrier and recognize the amyloid fibrils in the brain.

In this example several constructs that fulfil these requirements are described using knowledge for the construction of such a bi-head as is described often in the literature.

The bi-functional VHH molecule can have the following architectures:

Architecture for bi-functional bi-heads to dissolve amyloid fibrils:

### LEADER SEQUENCE-[FR1-CDR1-FR2-CDR2-FR3-CDR3-FR4]_{BBB}-LINKER-[FR1-CDR1-FR2-CDR2-FR3-CDR3-FR4]_{AMYLOID,β-42}

### LEADER SEQUENCE-[FR1-CDR1-FR2-CDR2-FR3-CDR3-FR4]_{AMYLOID-β42}-LINKER-[FR1-CDR1-FR2-CDR2-FR3-CDR3-FR4] _{BBB}

Architecture for bi-functional hi-heads that can be used for non-invasive imaging

### LEADER SEQUENCE-[FR1-CDR1-FR2-CDR2-FR3-CDR3-FR4]_{BBB}-LINKER-[FR1-CDR1-FR2-CDR2-FR3-CDR3-FR4]_{AMYLOID-β-42}-EXTENSION

### LEADER SEQUENCE-FR1-CDR1-FR2-CDR2-FR3-CDR3-FR4]_{AMYLOID-β-42}-LINKER-[FR1-CDR1-FR2-CDR2-FR3-CDR3-FR4]_{BBB}-EXTENSION

Amino acid sequences for VHH recognizing amyloid-β-42 are given in Table 5.3, whereas non-limiting examples of the amino acid sequences for VHH that recognize proteins on the endothelial cells of the Blood-Brain-Barrier (BBB), which interaction ensures translocation of the bi-functional VHH to the brain is given in Table 12

Leader sequences are necessary to produce the bi-functional VHH extracellularly. The host cell can be a bacteria, a lower eukaryote or a mammalian cell. All these host require different leader sequences well known in the art.

C-terminal extensions are preferred to ensure that the functionality of the bi-functional VHH is not impaired by the labeling, essential for non invasive imaging. Non-limiting examples of such extensions are:

His-His-His-His-His-His
His-His-Ala/Gly/Ser-Ala/Gly/Ser-Met-Ala/Gly/Ser-
Ala/Gly/Ser-His-His
Ala/Gly/Ser-Met-Ala/Gly/Ser
Ala/Gly/Ser/Cys-Ala/Gly/Ser
Cys-Ala/Gly/Ser

### BRIEF DESCRIPTION OF THE DRAWINGS

FIG. 1

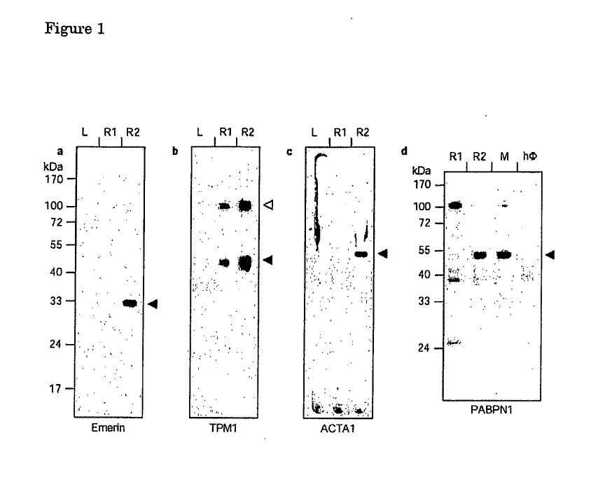

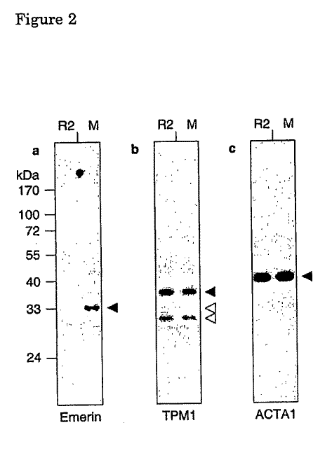

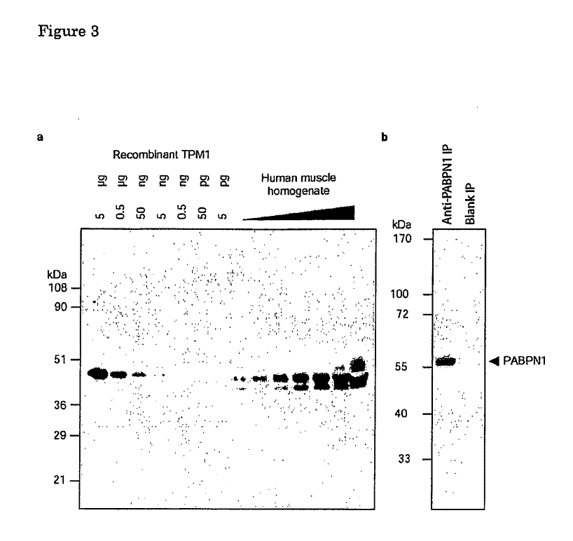

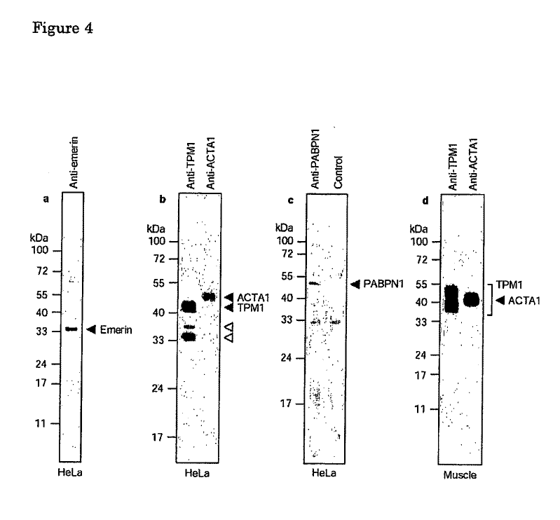

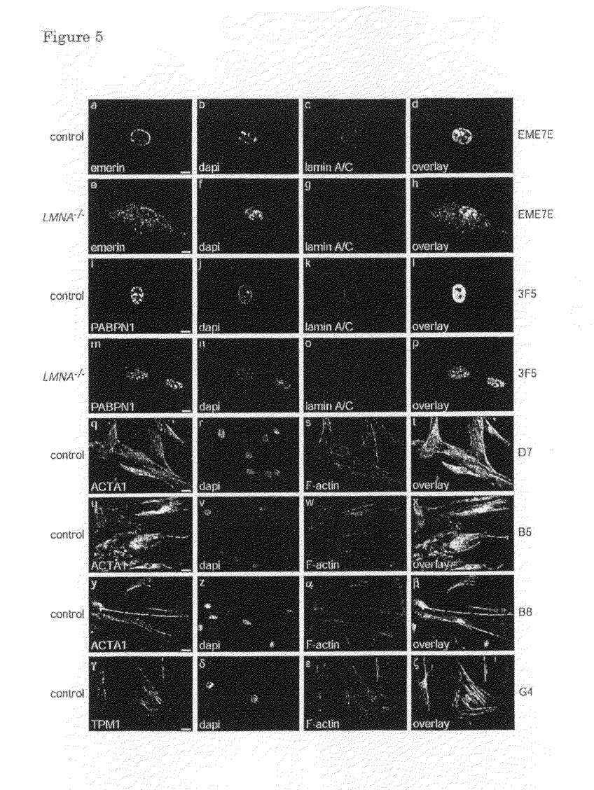

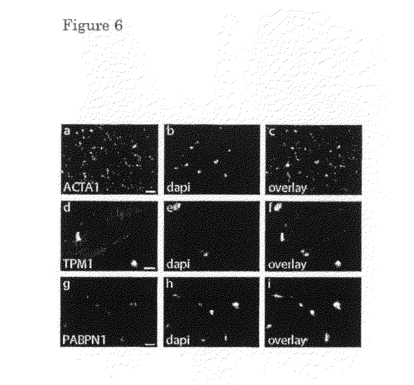

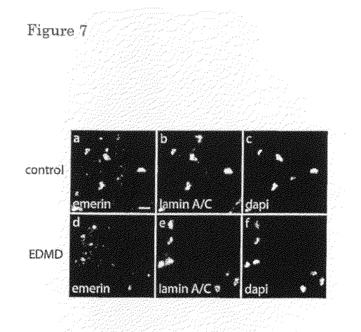

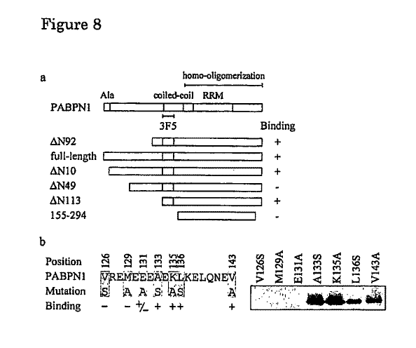

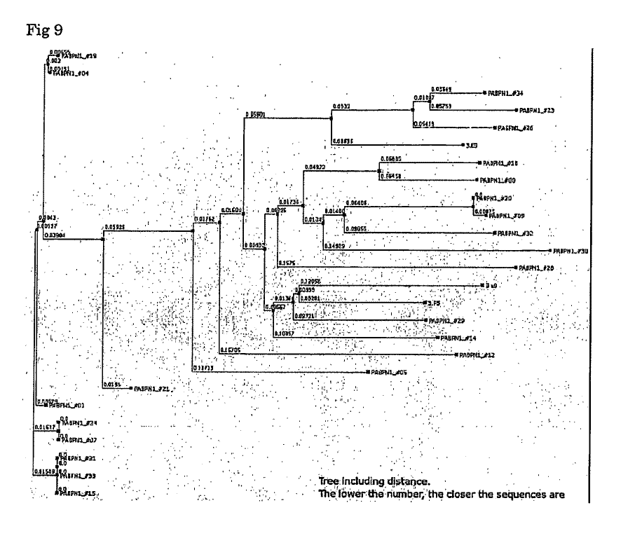

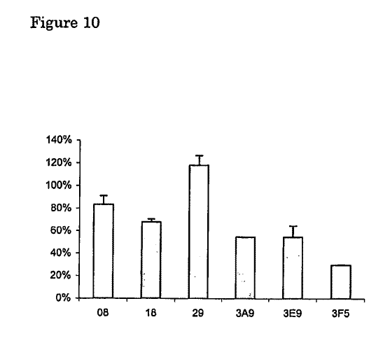

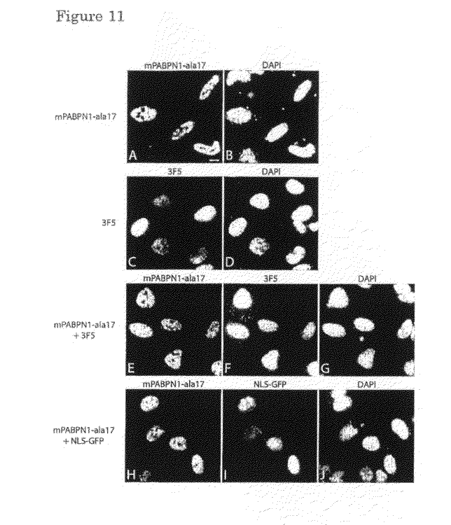

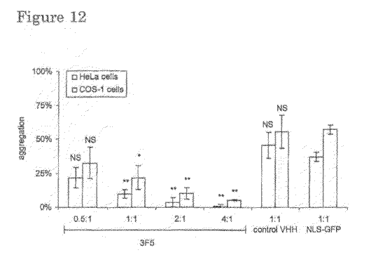

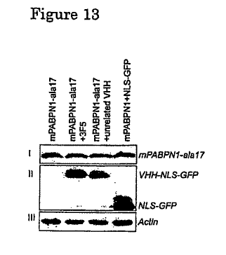

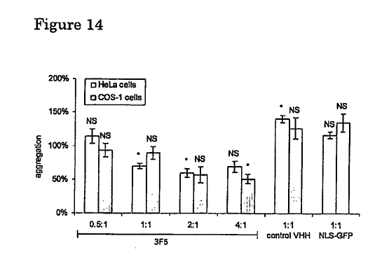

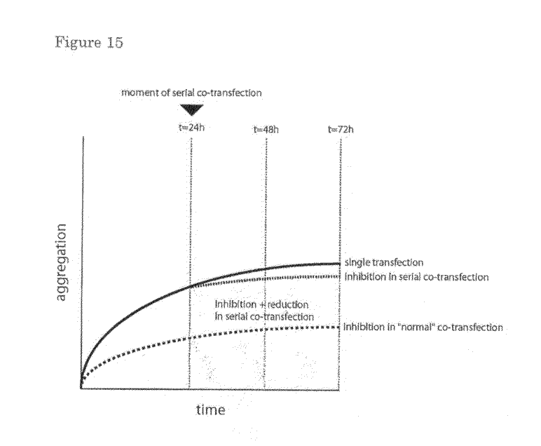

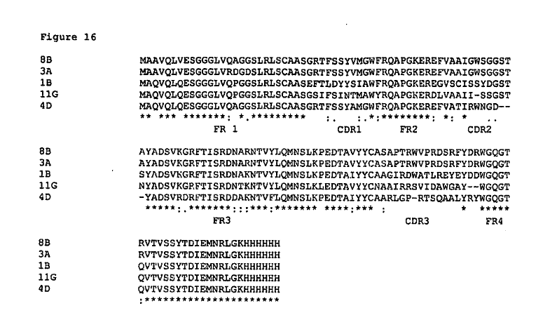

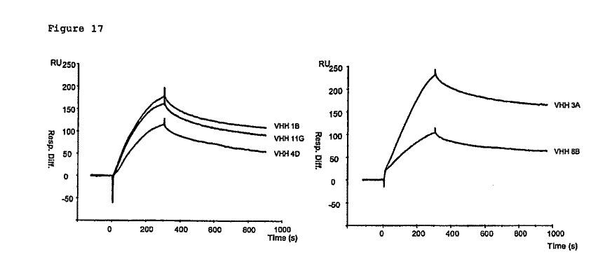

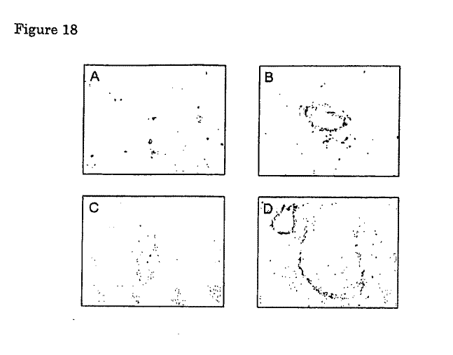

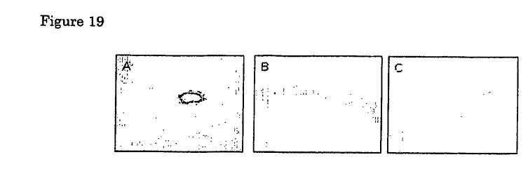

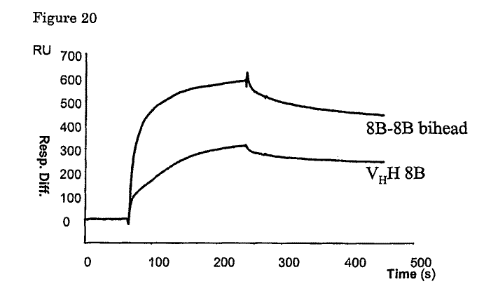

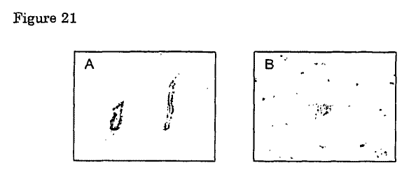

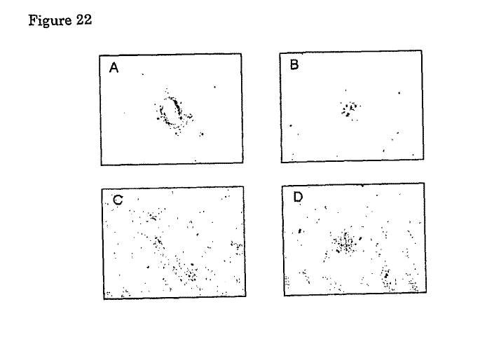

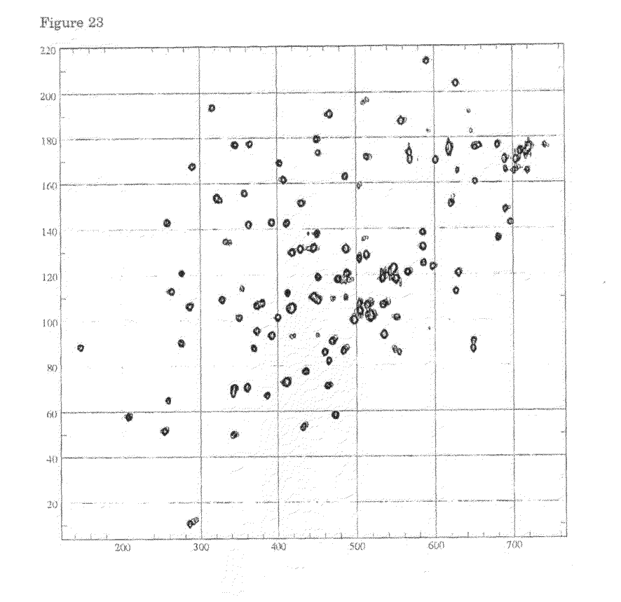

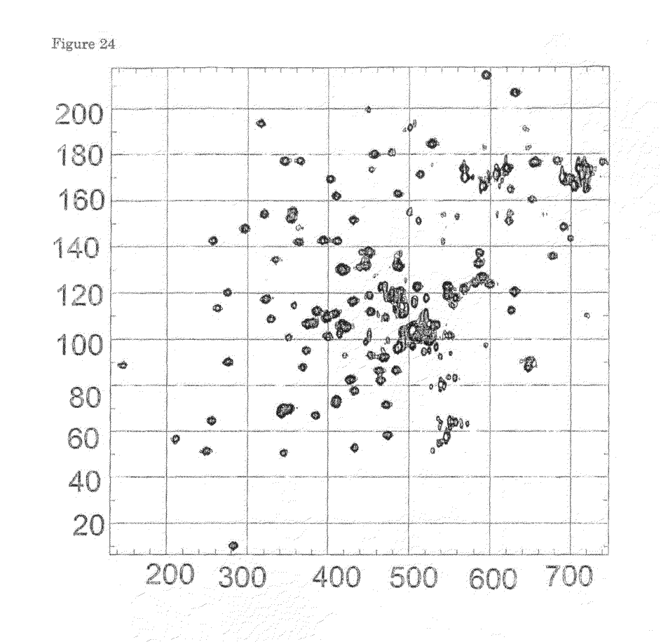

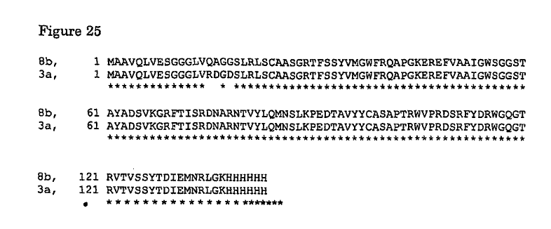

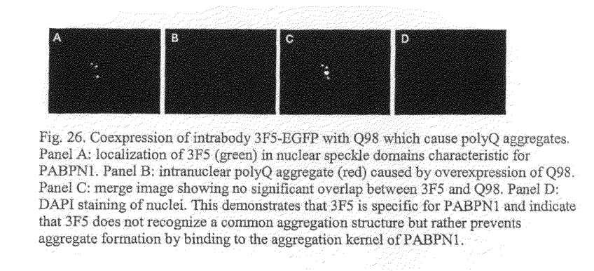

Monitoring of the selection progress with polyclonal phage antibodies and Western blotted recombinant antigens. Polyclonal phage antibodies from successive rounds of selection show an increased specificity for the target antigens. (a) After two rounds of selection emerin and (c) actin are specifically detected. (b)Tropomyosin-1 is already detected after one of round of selection. (d) In the case of PABPN1, HeLa cell extracts were used for selection monitoring. Incubation with helper phage without antibody fusion (hΦ) was included to reveal background bands resulting from the complex protein sample. L: Polyclonal phage antibodies from the input library; R1: Polyclonal phage antibodies after 1 round of selection; R2: Polyclonal phage antibodies after 2 rounds of selection. M: monoclonal phage. Arrowheads point to the target antigens, the open arrowhead (panel b) points to a multimer of the recombinant tropomyosin-1.

FIG. 2

Detection of endogenous antigens in a HeLa cell extract with polyclonal and monoclonal phage antibodies. (a) Emerin is detected with monoclonal phage (EME7E). (b) Polyclonal phage antibodies from the second round selection and monoclonal phage (G4) for tropomyosin-1 specifically bind tropomyosin-1. Multiple isoforms of tropomyosin-1 are visualized (open arrowheads). (c) Actin is detected with polyclonal phage antibodies from the second round selection and monoclonal phage (B8). R2: Polyclonal phage antibodies after 2 rounds of selection; M: monoclonal phage. Arrowheads point to the endogenous antigens.

FIG. 3

(a) Detection and quantification of tropomyosin-1 in tissue homogenates with polyclonal phage antibodies. Decreasing 10-fold dilutions of the recombinant antigen (left) are compared to 5-fold increasing amounts of human muscle homogenate (right). Less than 5 ng of recombinant tropomyosin-1 can be detected and less than 50 ng of endogenous antigen can be detected in the human muscle homogenate. (b) Immunoprecipitation (IP) of PABPN1 with anti-PABPN1 VHH (3F5), from HeLa extracts. In the blank IP lane no VHH was used. Arrowhead points to the endogenous PABPN1.

FIG. 4

Detection of endogenous antigens in complex protein samples with purified antibody fragments. (a, b, c) The isolated VHH for emerin, actin,tropomyosin-1 and PABPN1 bind their targets on Western blotted HeLa cell extract. In the PABPN1 panel (c), a combination of the secondary (anti-c-myc) and the tertiary (HRP-conjugated anti-Mouse IgG) antibodies used for VHH detection was applied in order to define background bands. (d) VHH specific for tropomyosin-1 and actin bind their targets in a muscle homogenate. Multiple isoforms of tropomyosin-1 are visualized (open arrowheads). Arrowheads point to the endogenous antigens.

FIG. 5

Application of VHH in immunofluorescence microscopy. Selected VHH were used as immunoprobes on human fibroblasts. VHH were applied at concentrations of 50 nM-500 nM. For the anti-emerin and anti-PABPN1 VHH control fibroblasts (a-d) and (i-1) respectively were compared to fibroblasts derived from a patient with a homozygous nonsense Y259X mutation in the LMNA gene causing complete absence of lamins A and C (e-h and m-p, respectively). Fibroblasts were incubated with VHH anti-emerin (EME7E) and anti-PABPN1 (3F5) (green channel) (a,e and i,m, respectively) and counterstained with DAN (blue chanel) (b,f and j,n respectively). Lamins A and C were detected with a polyclonal antibody against lamins A/C (red channel) (c,g and k,o). Dd,h,i,p are overlay images. Notice the absence of lamin in the red channel for the patient cells (g and o) and the dispersed staining for emerin apart from the nuclear lamina (e), while PABPN1 still localizes in the nuclear speckles (m).

Staining of control fibroblasts' cytoskeleton with three different anti-actin VHH (q, u, y) and with the anti-tropomyosin-1 VHH (y). Cells were counterstained with phalloidin (red channel) (s, w, α, ε) that marks F-actin in stress fibers and with DAPI (blue channel) (r, v, z, δ). Notice that different anti-actin VHH react with different structures of the cytoskeleton (q, u, y) while the anti-tropomyosin-1 VHH co-localizes with F-actin (ƒ). Overlays are shown (t, x, β, ƒ). Bars represent 5 μm (a, l, i, m) and 10 μm (q, u, y, γ).

FIG. 6

Application of VHH in immunohistochemistry. Selected VHH were used as immunoprobes on 7 μm cryosections from healthy human muscle. (a-c) Transverse sections of human muscle biopsies were incubated with anti-actin VHH (A2). (d-f) Longitudinal section with the anti-tropomyosin-1 VHH (G4). (g-i) Anti-PABPN1 μM (3F5) was used on transverse sections. Nuclei were stained with DAPI (panels b, e, h). Overlays are shown (c, f, i). Bars represent 10 μm.

FIG. 7

Emerin is absent in myonuclei of EDMD patients. (a-c) Control human muscle cryosections were incubated with (a) VHH anti-emerin (EME7E), (b) polyclonal antibody against lamins A/C and (c) dapi for nuclear staining. Emerin co-localized with lamins A/C in the nuclear membrane. (d-f) Muscle cryosections from an EDMD patient were incubated with (d) VHH anti-emerin (EME7e), (b) polyclonal antibody against lamins A/C and (c) dapi, revealing complete absence of emerin. Lamins A/C were detected in the nuclear membrane. Bar=10 μm.

FIG. 8

Epitope mapping of 3F5. (a) N-terminal deletions (□N10, □N49, □N92, □N113) and amino acids 155-294 of PABPN1 were Western blotted and incubated with 3F5. The N-terminal deletions with the exception of □N49 were recognized by 3F5. Although the □N49 protein was inconsistently not recognized by 3F5, the results with the other constructs pointed towards amino acids 113-155 to contain the epitope for 3F5. Ala: polyalanine stretch; coiled-coil: predicted coiled-coil domain; RRM: RNA-binding domain (b) Binding of 3F5 to different purified mutant proteins (V126S, M129A, E131A, A133S, K135A, L1365, V143A). 3F5 showed normal binding to point mutated proteins with substitutions between amino acids 135 and 143. In contrast, the V126S and M129A mutant proteins were not recognized by 3F5. In two independent experiments, reduced binding to the E131A mutant protein was observed. These results indicate that amino acids 126, 129 and 131 are involved in the epitope.

FIG. 9

Dendrogram based on amino acid sequence homology of VHHs recognizing PABPN1, that all pass the screening criteria applied [DNA fingerprinting, production in E. coli [not shown], Immunoprecipitation [FIG. 3], Immunofluorescent of endogenous PABPN1 [FIG. 6], and production in S. cerevisiae [data not shown]. The amino acid sequences were obtained via determination of the DNA sequences of the VHHs genes passing the screening tests. The Dendrogram depicts the distance [=amino acid variation of the selected and screened anti-PABPN1 VHHs] between the amino acid sequences according to standard procedures.

FIG. 10

Prevention of mutant PABPN1-ala17 aggregation in a cell model for OPMD. Different VHHs were co-expressed with mPABPN1. VHH 08, VHH 18 and VHH 29 were found using epitoop masking, using VHH 3F5 to mask the epitope recognized by VHH 3F5. VHH 29 binds definitely a different epitope, and proofed to be not able to prevent or dissolve PAPBN1 aggregates. VHH 3A and 3E9 recognize the same epitope but clearly are less efficient in prevention and dissolvement of mPABPN1 aggregates

FIG. 11

Prevention of mPABPN1-ala17 aggregation in HeLa cells. [a,b]: HeLa cells were transfected with VSV-tagged mPABPN1. Anti-VSV antibodies were used for mPABPN1 detection, intranuclear aggregates were observed 48 h after transfection. DAPI was used to visualize cell nuclei. [c,d]: HeLa cells were transfected with 3F5 in fusion with an NLS-sequence and GFP. The intrabody was produced and localized in cell nuclei 24 h after transfection. [e,f]: mPABPN1 and 3F5 were cotransfected in HeLa cells. mPABPN1 and the intrabody were detected in cell nuclei of co-tranfected cells. Decreased aggregation was observed with co-expression of 3F5 compared to control intrabody or NLS-GFP 48 h after transfection [FIG. 12]. [h,j] mPABPN1 and NLS-GFP were cotransfected in HeLa cells, mPABPN1 and NLS-GFP were detected in the cell nuclei. Intranuclear aqggregates were observed 48 h after transfection. Bar=10 μm

FIG. 12

Prevention of mPABPN1-ala17 aggregation in situ by 3F5 intrabody expression. mPABPN1 was co-transfected with 3F5 in different intrabody: mPABPN1 ratios. HeLa cells that co-express 3F5 show nuclear aggregation to only 10(+/−3) % at 1:1 ratio. 37(+/−) % of HeLa cells contain intranuclear aggregates with co-expression of NLS-GFP (1:1 ratio). A dose-dependent inhibitory effect of 3F5 co-transfection was observed. *p<0.05, **p<0.01, NS (not significant) p>0.05

FIG. 13

Protein levels of mPaBPN1-ala17 and 3F5 intrabody in single transfected and cotransfected cells. Cell lysates were analyzed by Western blotting [COS-1 cells 48 h after transfection).(I), mPABPN1 and (II) transfected intrabodies of control (NLS-GFP) were expressed at comparable levels. (III) The actin content was analyzed to investigated whether equal amounts of cells were analyzed.

FIG. 14

Dissolving existing mPABPN1-ala17 aggregates in situ demonstrated by serial transfection of m PABPN1 and 3F5. 3F5 was serially transfected in different intrabody: mPABPN1 ratios 24 h after transfection with mPABPN1. HeLa and

COS-1 cells transfected with mPABPN1 contained. 38 (+/−4) % and 33 (+/−8) % intranuclear aggregates 24 h post transfection, respectively. The aggregation at the moment of serial transfection was set to 100%. Double transfected cells were scored for the presence of intranuclear aggregates by microscopy 48 h after serial transfection. A dose-dependent decrease in the number of cells with intranuclear aggregates was observed. *p<0.05, NS (not significant p>0.05.

FIG. 16

Dose response of dissolving existing mPABPN1 aggregation according to methods given in FIG. 14 in HeLa and COS cells.

FIG. 16. Aligned amino acid sequence of VHHs selected against ε-amyloid. The frameworks 1-4 (FR) are depicted in blue; the CDR's 1-3 (Complementary Determining Region) are depicted in red.

FIG. 17. Composite sensorgrams illustrating binding of VHHs interacting with Aβ 1-40 and Aβ 1-42.

FIG. 18. Down syndrome, frontal cortex. Immunostaining of amyloid plaques (A) with anti-AβI-42 VHH 8B. HCHWA-D, frontal cortex. Immunostaining of arteriolar CAA with anti-Aβ1-42 VHH 8B (B) and VHH 3A (C). Alzheimers disease, frontal cortex. Immunostaining of amyloid deposition in menigeal artery with anti-Aβ1-42 VHH 3A (D).

FIG. 19. HCHWA-D, frontal cortex. Immunostaining of arteriolar CAA with anti-Aβ1-40 VHH 11G (A), 4D (B) and 1B (C). FIG. 8. Sensorgram illustrating binding of the homologous 8B-8B bihead (10 μg/ml) and VHH 8B (10 μg/ml) to immobilized. Aβ 1-42.

FIG. 20. Sensorgram illustrating binding of the homologous 8B-8B bihead (10 μg/ml) and VHH 8B (10 μg/ml) to immobilized Aβ 1-42.

FIG. 21. HCHWA-D, frontal cortex. Immunostaining of arteriolar CAA with homologous bihead 8B-8B (A). Down syndrome, frontal cortex. Immunostaining of amyloid plaque with homologous bihead 8B-8B (B).

FIG. 22. HCHWA-D, frontal cortex. Immunostaining of arteriolar CAA with bihead FC5-8B (A). Down syndrome, frontal cortex. Immunostaining of amyloid plaque with bihead FC5-8B (B). HCHWA-D, frontal cortex. Immunostaining of arteriolar CAA with bihead FC5-3A (C). Down syndrome, frontal cortex. Immunostaining of amyloid plaque with bihead FC5-3A (D).

FIG. 23. Overlay of 15N-1H HSQC spectra of 15N VHH-8B in the free form (black) and in the complex with bA 1-42 (0.8 and >1 equivalent, respectively in blue and red.¹.

FIG. 24. Overlay of 15N-1H HSQC spectra of 15N VHH-3A in the free form (black) and in the complex with bA 1-42>1 equivalent (red

FIG. 25. Comparison of the amino acid sequence between VHH 8B and VHH 3A.

FIG. 26. Coexpression of intrabody 3F6-EGFP with Q98.

TABLE 1

| | | |
|---|---|---|
| Gene | disease | OMIM |

Poly Gln diseases

| | | |
|---|---|---|
| IT15 | Huntington's disease (HD) | 143100 |
| AR | Kennedy's disease or spinal and bulbar | 313200 |
| | muscular atrophy | |
| ATXN1 | Spinocerebellar ataxia type 1c (SCA-1) | 164400 |
| ATXN2 | Spinocerebellar ataxia type 2 (SCA-2) | 183090 |
| ATXN3 | Machado-Joseph disease (MJD) or SCA-3 | 109150 |
| CACNA1A | Spinocerebellar ataxia type 6 | 183086 |
| ATXN7 | Spinocerebellar ataxia type 7 | 164500 |
| DRPLA | Dentatorubral-pallidoluysian atrophy | 125370 |
| TBP | Spinocerebellar ataxia type 17 (SCA-17) | 607136 |

Poly Ala diseases

| | | |
|---|---|---|
| HOXD13 | synpolydactyly type II | 186000 |
| RUNX2 | Cleidocranial dysplasia | 119600 |
| PABPN1 | oculopharyngeal muscular dystrophy | 164300 |
| ZIC2 | holoprosencephaly | |
| HOXA13 | hand foot genital syndrome | 140000 |
| FOXL2 | blepharophimosis, ptosis and epicanthus | 110100 |
| | inversus | |
| SOX3 | Mental retardation, X-linked with isolated | 300123 |
| | Growth hormone deficiency | |
| ARX | Infantile spasm syndrome, X-linked; | 308350 |
| | Partington syndrome; lissencephaly with | 309510 |
| | ambiguous genitalia, X-linked; mental | 300215 |
| | retardation X-linked 36 and 54 | 300430 |
| | | 300419 |
| PMX2B | Congenital central hypoventilation | 209880 |
| (PHOX2B) | syndrome/Ondine curse | |

RNA aggregation with commonalities to protein aggregation

| | | |
|---|---|---|
| DMPK | Myotonic dystrophy type 1 | 160900 |
| ZNF9 | Myotonic dystrophy type 2 | 602668 |

Aggregation disorders (partially overlapping with upper tables; from
Stefani M., Biochim Biophys Acta. 2004 Dec 24; 1739(1): 5-25)

| | |
|---|---|
| Alzheimer's disease | Aβ peptides (1-40, 1- |
| | 41, 1-42, 1-43); Tau |
| Spongiform encephalopathies | Prion protein (full- |
| | length or fragments) |
| Parkinson's disease | α-synuclein |
| | (wild type or mutant) |
| Fronto-temporal dementias | Tau (wild type or |
| | mutant) |
| Familial Danish dementia | ADan peptide |
| Familial British dementia | ABri peptide |
| Hereditary cerebral haemorrhage | Cystatin C |
| with amyloidoses | (minus a 10-residue fragment); |
| | Aβ peptides |
| Amyotrophic lateral sclerosis | Superoxide |
| | dismutase (wild type or mutant) |
| Dentatorubro-pallido-Luysian atrophy | Atrophin 1 |
| | (polyQ expansion) |
| Huntington disease | Huntingtin (polyQ |
| | expansion) |
| Cerebellar ataxias Ataxins | (polyQ |
| | expansion) |
| Kennedy disease Androgen receptor | (polyQ expansion) |
| Spino cerebellar ataxia 17 | (polyQ |
| TATA box-binding protein | expansion) |
| Primary systemic amyloidosis | Ig light chains (full- |
| | length or fragments) |
| Secondary systemic amyloidosis | Serum amyloid |
| | A (fragments) |
| Familial Mediterranean fever | Serum amyloid A |
| | (fragments) |
| Senile systemic amyloidosis | Transthyretin (wild- |
| | type or fragments thereof) |
| Familial amyloidotic polyneuropathy I | Transthyretin |
| | (over 45 variants or |
| | fragments thereof) |
| Hemodialysis-related amyloidosis | β2-microglobulin |
| Familial amyloid polyneuropathy III | Apolipoprotein A-1 |
| | (fragments) |
| Finnish hereditary systemic amyloidosis | Gelsolin (fragments |
| | of the mutant protein) |
| Type II diabetes | Pro-islet amyloid |
| | polypeptide (fragments) |
| Medullary carcinoma of the thyroid | Procalcitonin (full- |
| | length or fragment) |
| Atrial amyloidosis | Atrial natriuretic |
| | factor |
| Lysozyme systemic amyloidosis | Lysozyme |
| | (full-length, mutant) |
| Insulin-related amyloid | Insulin (full-length) |
| Fibrinogen α-chain amyloidosis | Fibrinogen (α-chain |
| | variants and fragments) |

TABLE 2

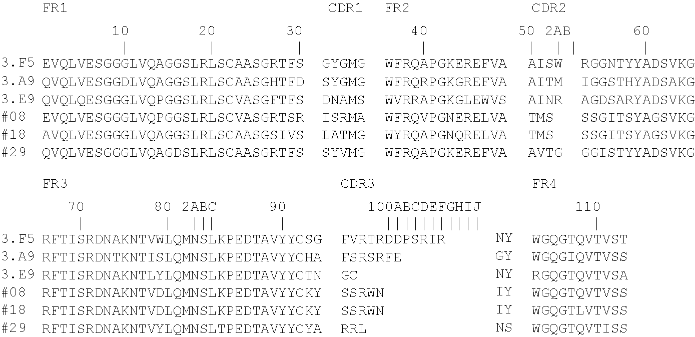

TABLE 3
Hallmark Residues in VHH

| Position | Human V_{H}3 | Hallmark Residues |
|---|---|---|
| 11 | L, V; predominantly L | L, M, S, V,W; preferably L |
| 37 | V, I, F; usually V | F⁽¹⁾, Y, H, I, L or V, preferably F⁽¹⁾ or Y |
| 44⁽⁸⁾ | G | G⁽²⁾, E⁽³⁾, A, D, Q, R, S, L; |
| | | preferably G⁽²⁾, E⁽³⁾ or Q; |
| | | most preferably G⁽²⁾ or E⁽³⁾. |
| 45⁽⁸⁾ | L | L⁽²⁾, R⁽³⁾, C, I, L, P, Q, V; preferably L⁽²⁾ or R⁽³⁾ |
| 47⁽⁸⁾ | W, Y | W⁽²⁾, L⁽¹⁾ or F⁽¹⁾, A, G, I, M, R, S, V or Y; preferably |
| | | W⁽²⁾, L⁽¹⁾, F⁽¹⁾ or R |
| 83 | R or K; usually R | R, K⁽⁵⁾, N, E⁽⁵⁾, G, I, M, Q or T; preferably K or R; |
| | | most preferably K |
| 84 | A, T, D; predominantly A | P⁽⁵⁾, A, L, R, S, T, D, V; preferably P |
| 103 | W | W⁽⁴⁾, P⁽⁶⁾, R⁽⁶⁾, S; preferably W |
| 104 | G | G or D; preferably G |
| 108 | L, M or T; predominantly L | Q, L⁽⁷⁾ or R; preferably Q or L⁽⁷⁾ |

Notes:
⁽¹⁾In particular, but not exclusively, in combination with KERE or KQRE at positions 43-46.
⁽²⁾Usually as GLEW at positions 44-47.
⁽³⁾Usually as KERE or KQRE at positions 43-46, e.g. as KEREL, KEREF, KQREL, KQREF or KEREG at positions 43-47. Alternatively, also sequences such as TERE (for example TEREL), KECE (for example KECEL or KECER), RERE (for example REREG), QERE (for example QEREG), KGRE (for example KGREG), KDRE (for example KDREV) are possible. Some other possible, but less preferred sequences include for example DECKL and NVCEL.
⁽⁴⁾With both GLEW at positions 44-47 and KERE or KQRE at positions 43-46.
⁽⁵⁾Often as KP or EP at positions 83-84 of naturally occurring V_{HH} domains.
⁽⁶⁾In particular, but not exclusively, in combination with GLEW at positions 44-47.
⁽⁷⁾With the proviso that when positions 44-47 are GLEW, position 108 is always Q.
⁽⁸⁾The GLEW group also contains GLEW-like sequences at positions 44-47, such as for example GVEW, EPEW, GLER, DQEW, DLEW, GIEW, ELEW, GPEW, EWLP, GPER, GLER and ELEW.

TABLE 5.1
Some preferred but non-limiting combinations of Hallmark Residues
in naturally occurring VHH. For humanization of these combinations,
reference is made to the specification.

| | 11 | 37 | 44 | 45 | 47 | 83 | 84 | 103 | 104 | 108 |
|---|---|---|---|---|---|---|---|---|---|---|
| DP-47 (human) | M | V | G | L | W | R | A | W | G | L |
| “KERE” group | L | F | E | R | L | K | P | W | G | Q |
| | L | F | E | R | F | E | P | W | G | Q |
| | L | F | E | R | F | K | P | W | G | Q |
| | L | Y | Q | R | L | K | P | W | G | Q |
| | L | F | L | R | V | K | P | Q | G | Q |
| | L | F | Q | R | L | K | P | W | G | Q |
| | L | F | E | R | F | K | P | W | G | Q |
| “GLEW” group | L | V | G | L | W | K | S | W | G | Q |
| | M | V | G | L | W | K | P | R | G | Q |

TABLE 5.2

| | | Cross | | |
|---|---|---|---|---|
| Antibody | | reactivity to | | Immunohistochemistry |
| fragment | ELISA | A-beta-40 | Western blot | on HCHWAD sections |
| 2F | ++ | In progress | + | ++ |
| 3A | + | In progress | ++ | ++ |
| 6B | + | — | + | ++/− |
| 7B | ++ | — | ++ | +++ |
| 8B | + | In progress | ++ | +++/− |
| 11D | + | — | ++ | + |

TABLE 5.3

| | |
|---|---|
| | 1 10 20 30 40 |
| 1B | Q V Q L Q E S G G G L V Q A G G S L R L S C A A S G R T F S S Y V M G W F R Q A |
| 1D | Q V Q L Q D S G G G L V Q A G G S L R L S C A A S G R T F S N Y A M G W F R Q A |
| 2A | Q V Q L Q D S G G G L V Q A G G S L R L S C A A S G R T F S S Y V M G W F R Q A |
| 2B | Q V K L E E S G G G L V Q P G G S L R L S C A A S G R T R T I R A M A W F R Q A |
| 2F | Q V Q L Q E S G G G L V Q A G G S L R L S C A A S G R T F S S Y V M G W F R Q A |
| 3A | A V Q L V E S G G G L V R D G G S L R L S C A A S G R T F S S Y V M G W F R Q A |
| 3H | Q V Q L Q E S G G G L V Q A G G S L R L S C T A S G R T F P S W A M A W F R Q A |
| 4C | Q V Q L Q E S G G G L V Q A G G S L R L S C A A S G R T F S R Y A M G W F R Q A |
| 8F | Q V Q L Q D S G G G L V Q A G G S L R L S C A A S G R T F S S L V M G W F R Q A |
| 11D | A V Q L V E S G G G L V Q A G G S L R L S C A A S Q R T F S S Y V M G W F R Q A |
| | 41 50 2a 60 70 |
| 1B | P G K E R E F V A A I G W S G G S T A Y A D S V K G R F T I S R D D A R N T V Y |
| 1D | P E K E R E F V A T I N G S G R S T Y Y A D S V K G R F T I S R D N A K N T V Y |
| 2A | P G K E R E F V A A I G W S G G S T A Y A D S V K G R F T I S R D N A R N T V Y |
| 2B | L G K Q R E F I A R I T V G G T N Y A D S V K D R F T I S R D N A K N T V Y |
| 2F | P G K E R E F V A A I G W S G G S T A Y A D S V K G R F T I S R D N A R N T V Y |
| 3A | P G K E R E F V A A I G W S G G S T A Y A D S V K G R F T I S R D N A R N T V Y |
| 3H | P G K E R E F L A A S W S G G N T A Y A N S V K G R F T I S R D N A K N T V Y |
| 4C | P G K E R E F V A R I R W S G G S T Y Y A D S V K G R F T I S R D N A K N T V Y |
| 8F | S G K E R E F V A A I G W S G G S T A Y A D S V K G R F T I S R D N A R N T V Y |
| 11D | P G K E R E F V A A I G W S G G S T A Y A D S V K G R F T I S R D N A R N T V Y |
| | 50 2a b c 90 103 110 |
| 1A | L Q M N S L K P E D T A V Y Y C A S A P T R W V P R D S R F Y D R W G Q G T Q V T V S S |
| 1D | L Q M N S L K P E D T A V Y Y C A S K L Y G S G T P R D T G D K Y S N W G Q G T Q V T V S S |
| 2A | L Q M N S L K P E D T A V Y Y C A S A P T R W V P R D S R F Y D R W G Q G T Q V T V S S |
| 2B | L Q M N S L K P E D T A V Y Y C Y S K T W G G R N Y W G Q G T Q V T V P S |
| 2F | L Q M N S L K P E D T A V Y Y C A S A P T R W V P R D S R F Y D R W G Q G T Q V T V S S |
| 3A | L Q M N S L K P E D T A V Y Y C A S A P T R W V P R D S R F Y D R W G Q G T R V T V S S |
| 3H | L Q M N S L K P E D T A V Y Y C A T G T T R T V L A A S Y D Y W G Q G T Q V T V S S |
| 4C | L Q M N S L K P E D T A V Y Y C A A G G T S Q L S F D Y W G Q G T Q V T V S S |
| 8F | L Q M N S L K P E D T A V Y Y C A S A P T R W V P R D S R F Y D R W G Q G T Q V T V S S |
| 11D | L Q M N S L K P E D T A V Y Y C A S A P T R W V P R D S R F Y D R W G Q G T R V T V S S |

TABLE 6
Non-limiting examples of amino acid residues in FR1 (for the
footnotes, see the footnotes to Table 2)

| | Amino acid residue(s): | | V_{HH} | V_{HH} |
|---|---|---|---|---|
| Pos. | Human V_{H}3 | Camelid V_{HH}'s | Ent. | Var. |
| 1 | E, Q | Q, A, E | — | — |
| 2 | V | V | 0.2 | 1 |
| 3 | Q | Q, K | 0.3 | 2 |
| 4 | L | L | 0.1 | 1 |
| 5 | V, L | Q, E, L, V | 0.8 | 3 |
| 6 | E | E, D, Q, A | 0.8 | 4 |
| 7 | S, T | S, F | 0.3 | 2 |
| 8 | G, R | G | 0.1 | 1 |
| 9 | G | G | 0 | 1 |
| 10 | G, V | G, D, R | 0.3 | 2 |
| 11 | Hallmark residue: L, M, S, V, W; preferably L | | 0.8 | 2 |
| 12 | V, I | V, A | 0.2 | 2 |
| 13 | Q, K, R | Q, E, K, P, R | 0.4 | 4 |
| 14 | P | A, Q, A, G, P, S, T, V | 1 | 5 |
| 15 | G | G | 0 | 1 |
| 16 | G, R | G, A, E, D | 0.4 | 3 |
| 17 | S | S, F | 0.5 | 2 |
| 18 | L | L, V | 0.1 | 1 |
| 19 | R, K | R, K, L, N, S, T | 0.6 | 4 |
| 20 | L | L, F, I, V | 0.5 | 4 |
| 21 | S | S, A, F, T | 0.2 | 3 |
| 22 | C | C | 0 | 1 |
| 23 | A, T | A, D, E, P, S, T, V | 1.3 | 5 |
| 24 | A | A, I, L, S, T, V | 1 | 6 |
| 25 | S | S, A, F, P, T | 0.5 | 5 |
| 26 | G | G, A, D, E, R, S, T, V | 0.7 | 7 |
| 27 | F | S, F, R, L, P, G, N, | 2.3 | 13 |
| 28 | T | N, T, E, D, S, I, R, A, G, R, F, Y | 1.7 | 11 |
| 29 | F, V | F, L, D, S, I, G, V, A | 1.9 | 11 |
| 30 | S, D, G | N, S, E, G, A, D, M, T | 1.8 | 11 |

TABLE 7
Non-limiting examples of amino acid residues in FR2 (for the
footnotes, see the footnotes to Table 3)

| | Amino acid residue(s): | | V_{HH} | V_{HH} |
|---|---|---|---|---|
| Pos. | Human V_{H}3 | Camelid V_{HH}'s | Ent. | Var. |
| 36 | W | W | 0.1 | 1 |
| 37 | Hallmark residue: F⁽¹⁾, H, I, L, Y or V, | | 1.1 | 6 |
| | preferably F⁽¹⁾ or Y | | | |
| 38 | R | R | 0.2 | 1 |
| 39 | Q | Q, H, P, R | 0.3 | 2 |
| 40 | A | A, F, G, L, P, T, V | 0.9 | 7 |
| 41 | P, S, T | P, A, L, S | 0.4 | 3 |
| 42 | G | G, E | 0.2 | 2 |
| 43 | K | K, D, E, N, Q, R, T, V | 0.7 | 6 |
| 44 | Hallmark residue: G⁽²⁾, E⁽³⁾, A, D, Q, R, | | 1.3 | 5 |
| | S, L; preferably G⁽²⁾, E⁽³⁾ or | | | |
| | Q; most preferably G⁽²⁾ or E⁽³⁾. | | | |
| 45 | Hallmark residue: L⁽²⁾, R⁽³⁾, C, I, L, P, | | 0.6 | 4 |
| | Q, V; preferably L⁽²⁾ or R⁽³⁾ | | | |
| 46 | E, V | E, D, K, Q, V | 0.4 | 2 |
| 47 | Hallmark residue W⁽²⁾, L⁽¹⁾ or F⁽¹⁾, A, G, I, | | 1.9 | 9 |
| | M, R, S, V or Y; preferably W⁽²⁾, L⁽¹⁾, F⁽¹⁾ or R | | | |
| 48 | V | V, I, L | 0.4 | 3 |
| 49 | S, A, G | A, S, G, T, V | 0.8 | 3 |

TABLE 8
Non-limiting examples of amino acid residues in FR3 (for the
footnotes, see the footnotes to Table 3)

| | Amino acid residue(s): | | V_{HH} | V_{HH} |
|---|---|---|---|---|
| Pos. | Human V_{H}3 | Camelid V_{HH}'s | Ent. | Var. |
| 66 | R | R | 0.1 | 1 |
| 67 | F | F, L, V | 0.1 | 1 |
| 68 | T | T, A, N, S | 0.5 | 4 |
| 69 | I | I, L, M, V | 0.4 | 4 |
| 70 | S | S, A, F, T | 0.3 | 4 |
| 71 | R | R, G, H, I, L, K, Q, S, T, W | 1.2 | 8 |
| 72 | D, E | D, E, G, N, V | 0.5 | 4 |
| 73 | N, D, G | N, A, D, F, I, K, L, R, S, T, V, Y | 1.2 | 9 |
| 74 | A, S | A, D, G, N, P, S, T, V | 1 | 7 |
| 75 | K | K, A, E, K, L, N, Q, R | 0.9 | 6 |
| 76 | N, S | N, D, K, R, S, T, Y | 0.9 | 6 |
| 77 | S, T, I | T, A, E, I, M, P, S | 0.8 | 5 |
| 78 | L, A | V, L, A, F, G, I, M | 1.2 | 5 |
| 79 | Y, H | Y, A, D, F, H, N, S, T | 1 | 7 |
| 80 | L | L, F, V | 0.1 | 1 |
| 81 | Q | Q, E, I, L, R, T | 0.6 | 5 |
| 82 | M | M, I, L, V | 0.2 | 2 |
| 82a | N, G | N, D, G, H, S, T | 0.8 | 4 |
| 82b | S | S, N, D, G, R, T | 1 | 6 |
| 82c | L | L, P, V | 0.1 | 2 |
| 83 | Hallmark residue: R, K⁽⁵⁾, N, E⁽⁵⁾, G, I, M, | | 0.9 | 7 |
| | Q or T; preferably K or R; most preferably K | | | |
| 84 | Hallmark residue: P⁽⁵⁾, A, D, L, R, | | 0.7 | 6 |
| | S, T, V; preferably P | | | |
| 85 | E, G | E, D, G, Q | 0.5 | 3 |
| 86 | D | D | 0 | 1 |
| 87 | T, M | T, A, S | 0.2 | 3 |
| 88 | A | A, G, S | 0.3 | 2 |
| 89 | V, L | V, A, D, I, L, M, N, R, T | 1.4 | 6 |
| 90 | Y | Y, F | 0 | 1 |
| 91 | Y, H | Y, D, F, H, L, S, T, V | 0.6 | 4 |
| 92 | C | C | 0 | 1 |
| 93 | A, K, T | A, N, G, H, K, N, R, S, T, V, Y | 1.4 | 10 |
| 94 | K, R, T | A, V, C, F, G, I, K, L, R, S or T | 1.6 | 9 |

TABLE 9
Non-limiting examples of amino acid residues in FR4 (for the
footnotes, see the footnotes to Table 3)

| | | |
|---|---|---|
| Amino acid residue(s): | V_{HH} | V_{HH} |

| | | | | |
|---|---|---|---|---|
| Pos. | Human V_{H}3 | Camelid V_{HH}'s | Ent. | Var. |

| | | | |
|---|---|---|---|
| 103 | Hallmark residue: W⁽⁴⁾, | 0.4 | 2 |
| | P⁽⁶⁾, R⁽⁶⁾, | | |
| | S; preferably W | | |
| 104 | Hallmark residue: G or D; | 0.1 | 1 |
| | preferably G | | |

| | | | | |
|---|---|---|---|---|
| 105 | Q, R | Q, E, K, P, R | 0.6 | 4 |
| 106 | G | G | 0.1 | 1 |
| 107 | T | T, A, I | 0.3 | 2 |

| | | | |
|---|---|---|---|
| 108 | Hallmark residue: Q, L⁽⁷⁾ or R; | 0.4 | 3 |
| | preferably Q or L⁽⁷⁾ | | |

| | | | | |
|---|---|---|---|---|
| 109 | V | V | 0.1 | 1 |
| 110 | T | T, I, A | 0.2 | 1 |
| 111 | V | V, A, I | 0.3 | 2 |
| 112 | S | S, F | 0.3 | 1 |
| 113 | S | S, A, L, P, T | 0.4 | 3 |

TABLE 10
Amino acid sequences of the isolated VHH for emerin. Genetically different clones identified by DNA fingerprinting that showed binding to
the recombinant emerin were sequenced. Sequences are compared to the best performing clone in diverse immunological techniques, VHH EME7E.

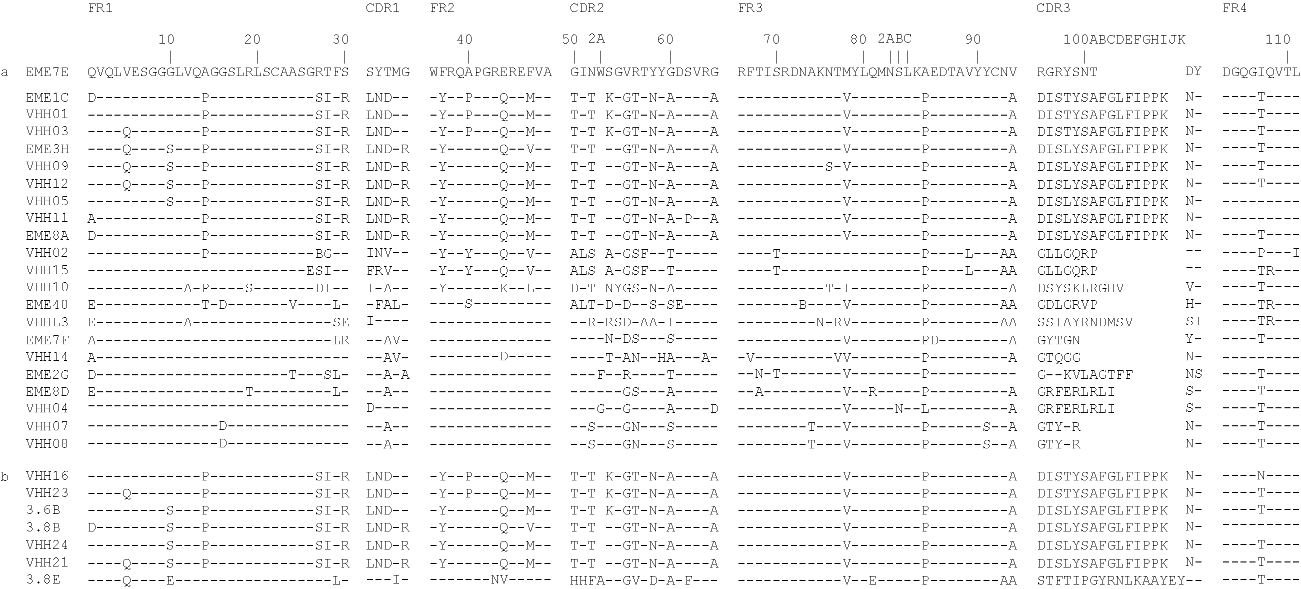

a VHH that were obtained by selection using 1^{st} round capturing and 2^{nd} round biopanning (n = 95).
b VHH that were obtained by selection using 1^{st} round biopanning and 2^{nd} round capturing (n = 95). The sequences are numbered according to Kabat et al. [31] Annotation includes assignment of framework regions (FR) and complementarity determining regions (CDR).

TABLE 11
VHH against PABPN1 from capturing/panning selections

| | | |
|---|---|---|
| 3F5 | BIAcore affinity measurements: | 5 nM |
| 3A9 | BIAcore affinity measurements: | 8 nM |
| 3E9 | BIAcore affinity measurements: | 14 nM |

VHH against PABPN1 selected by epitope-masking against
native PABPN1.

| | | |
|---|---|---|
| 08 | BIAcore affinity measurements: | 57 nM |
| 18 | BIAcore affinity measurements: | 7 nM |
| 29 | BIAcore affinity measurements: | 11 nM |

TABLE 12
BBB1
1 10 20 30 40
EVQ LQA SGG G LVQ AGG SLR L SCA ASG FKI T HYT MGW FRQ A
41 50 a 60 70 80
PGK ERE FVS R ITW GGD NTF Y SNS VKG RFT I SRD NAK NTV YL
81 a bc 90 100 abc d e 110 120
QMN SLK PED T ADY YCA AGS T STA TPL RVD Y WGK GTQ VTV S S
BBB2
1 10 20 30 40
EVQ LQA SGG G LVQ AGG SLR L SCS ASV RTF S IYT MGW FRQ A
41 50 a 60 70 80
PGK ERE FVA G INR SGD VTK Y ADF VKG RFS I SRD HAK NMV YL
81 90 100 a b c d ef g h 113 120
QMN SLK PED T ALY YCA ATW A YDT VGA LTS G YNF WGQ GTQ V TVS S

Two preferred amino acid sequences of VHH domains which can translocate via Blood Brain Barrier to the brain.

The underlined amino acids represent the CDRs. Substitution of one or two amino acids of the CDR's by the amino acid indicated in my previous table may have also the property to pass the Blood Brain Barrier as well. Substitutions of the Frame work residues to improve the functional and biophysical properties of the VHH domains are desired. However the substitutions should be restricted to those mentioned to amino acid at any position as given in the Entropy Variability tables 6-9.

TABLE 13

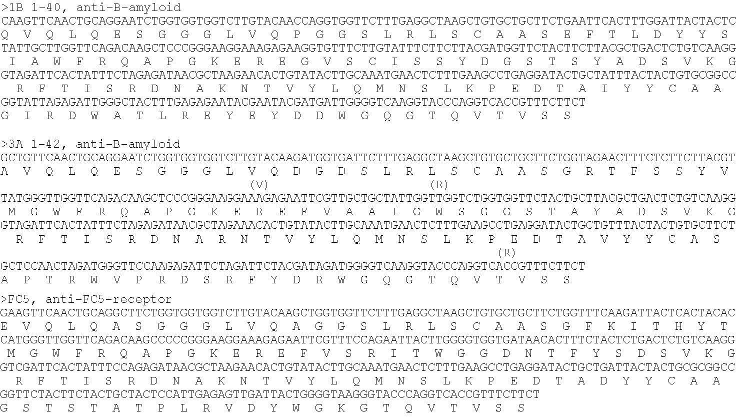

TABLE 14

| VHH 07 | QVQLQESGGGLVQPGGSLRLSCAASRFTLDNYAVGWFRQAAGKEREAVSCSSSGDGRTYYADSVKGRFTISRDNAKDTVYLQMNSLKPEDTAV |
|---|---|
| VHH 24 | QVQLQESGGGLVQPGGSLRLSCAASRFTLDNYAVGWFRQAAGKEREAVSCSSSGDGRTYYADSVKGRFTISRDNAKDTVYLQMNSLKPEDTAV |
| VHH 15 | QVQLQESGGGAVQPGGSLRLSCAASRFTLDNYAVGWFRQAPGKEREAVSCSSSSDGRTYYADSVKGRFTISRDNAKDTVYLQMNSLKPEDTAV |
| VHH 33 | QVQLQESGGGAVQPGGSLRLSCAASRFTLDNYAVGWFRQAPGKEREAVSCSSSSDGRTYYADSVKGRFTISRDNAKDTVYLQMNSLKPEDTAV |
| VHH 31 | QVQLQESGGGAVQPGGSLRLSCAASRFTLDNYAVGWFRQAPGKEREAVSCSSSSDGRTYYADSVKGRFTISRDNAKDTVYLQMNSLKPEDTAV |
| VHH 01 | QVQLQESGGGLVQPGGSLRLSCAASRFTLDNYAVGWFRQAPGKEREAVSCSSSSDGPTYYADSVKGRFTISRDNAKDTVYLQMNSLKPEDTAV |
| VHH 04 | QVQLQESGGGLVQPGGSLRLSCAASRFTLDYYAVGWFRQAPGKEREAVSCSSSSDGRTYYADSVKGRFTISRDNAKDTVYLQMNSLKPEDTAV |
| VHH 19 | QVQLQESGGGLVQPGGSLRLSCAASRFTLDYYAVGWFRQAPGKEREAVSCSSSSDGHTYYADSVKGRFTISRDNAKDTYVLQMNSLKPEDTAV |
| VHH 21 | AVQLVESGGGLVQAGGSLRLSCAASGFTFDYYAVGWFRQAPGKEREAVSCSSSSDGRTYYADSVKGRFTISRDNAKDTVYLQMNSLKPEDTAV |
| VHH 05 | AVQLVESGGGLVQAGGSLRLSCAASGFTFDDYAIGWFRQAPGKEREGISCISKSDGNTNYADSVKGRFTISSDNAKNTVFLQMNSLKPEDTAV |
| VHH 23 | AVQLVESGGDLVQPGGSLRLSCTASGFTFNTYGMSWVRQAPGKGLVWVSSISSGG-VPMYADSVKGRFTISRDNTKNTLYLQMNSLKPEDTAV |
| VHH 34 | QVQLQESGGGLVQPGGSLRLSCAASGFTFSTYGMSWVRQAPGKGLRWVSSISSGG-VPMYASSVKGRFTISRDNAKNTLYLQMNSLKPEDTAV |
| VHH 26 | QVQLQESGGGLVQPGGSLRLSCAASGFTFSTYGMSWVRQAPGKGVEWVSSINSGGVVPMYAASVKGRFTISRDNANNTLTLQMNSLKPEDTAV |
| 3.E9 | QVQLQESGGGLVQPGGSLRLSCVASGFTFSDNAMSWVRRAPGKGLEWVSAINRAGDSARYADSVKGRFTISRDNAKNTLYLQMNSLKPEDTAV |
| VHH 08 | EVQLVESGGGLVQPGGSLRLSCVASGRTSRISRMAWFRQVPGNERELVATMS-SSGITSYAGSVKGRFTISRDNAKNTVDLQMNSLKPEDTAV |
| VHH 18 | AVQLVESGGGLVQAGGSLRLSCAASGSIVSLATMGWYRQAPGNQRELVATMS-SSGITSYAGSVKGRFTYSRDNAKNTVDLQMNSLKPEDTAV |
| VHH 09 | QVQLQDSGGGLVQPGGSLRLSCAASGSIFSFSTMGWYRQAPGKQRELVAAIT-RTGTTNYADSVKGRFTISRDNTKNTVYLQMNSLKPEDTSA |
| VHH 20 | QVQLQESGGGLVQPGGSLRLSCAASGSIFSFSTMGWYRQAPGKQRELVAAIT-RTGTTNYADSVKGRFTISRDNTKNTVYLQMNSLKPEDTSA |
| VHH 32 | EVQLVESGGGLVQPGGSLRLSCAASGSIFRINTMAWYRQAPGKQRELVATIT-NGGNTNYADSVKGRFTISRDNAKNTVYLQMNSLKPEDTAV |
| VHH 30 | EVQLVESGGGLVQPGGSLRLSCAGSLDIFSVYVMGWYRQVPGGQRDLVATIT-KEGMPDYADSVKGRFTISRDNAENTVYLQMNSLKPEDTGV |
| VHH 28 | QVQLQESGGGLVQAGGSLRLSCAASGLAFSRTAMAWFRQAPGKEREFVAAIAWSAGNTLYEESVKGRFTISRDNAKNTVYLQMNNLKPEDTAV |
| VHH 29 | QVQLVESGGGLVQAGDSLRLSCAASGRTFSSYVMGWFRQAPGKEREFVAAVTGGGISTYYADSVKGRFTISRDNAKNTVYLQMNSLTPEDTAV |
| 3.F5 | EVQLVESGGGLVQAGGSLRLSCAASGRTFSGYGMGWFRQAPGKEREFVAAISWRGGNTYYADSVKGRFTISRDNAKNTVWLQMNSLKPEDTAV |
| 3.A9 | QVQLVESGGDLVQAGGSLRLSCAASGHTFDSYGMGWFRQRPGKGREFVAAITMIGGSTHYADSAKGRFTISRDNTKNTISLQMNSLKPEDTAV |
| VHH 14 | QVQLQESGGGLVQAGGSLRLSCTASGHTLSSYAMGWFRQAPGKEREFVAAIS-WSDITYYEDSVKGRFTISRDNAKNTVYLQMNSLNPEDTAV |
| VHH 12 | QVQLQESGGGLVQAGGSLRLSCTVSGFTVDAYAMGWRRQTPGKGHEVVACISSPDGITTYADSVKGRFTISRDSVQNTVSLQMDSLKPEDTAV |
| VHH 07 | YYCATGF-----FVRSCSSPD---SYDYWGQGTQVIVSS |
| VHH 24 | YYCATGF-----FVRSCSSPD---SYDYWGQGTQVIVSS |
| VHH 15 | YYCATGF-----FVRSCRSPD---SYDYWGQGTQVIVSS |
| VHH 33 | YYCATGF-----FVRSCRSPD---SYDYWGQGTQVIVSS |
| VHH 31 | YYCATGF-----FVRSCRSPD---SYDYWGQGTQVIVSS |
| VHH 01 | YYCATGF-----FVRSCSSPD---SYDYWGQGTQVIVSS |
| VHH 04 | YYCATGF-----FVRSCSSPF---SYDYWGQGTQVIVSS |
| VHH 19 | YYCATGF-----FVRSCSSPD---SYDYWGQGTQVIVSS |
| VHH 21 | YYCATGF-----FVRSCSSPD---SYDYWGQGTRVTVSS |
| VHH 05 | YYCAVGV-----LG-SCPIMVYL-LYDDWGQGTRVTVSS |
| VHH 23 | YYCVKGY-----YTGSF---------LPPGQGTRVTVSS |
| VHH 34 | YYCVKGY-----YTGDF---------LPPGQGTQVAVSS |
| VHH 26 | YYCVKGY-----YTGN----------PPLGQGTQVTVSS |
| 3.E9 | YYCTNG--------GN-----------YRGQGTQVTVSA |
| VHH 08 | YYCK--------YSSRWN--------IYWGQGTQVTVSS |
| VHH 18 | YYCK--------YSSRWN--------IYWGQGTLVTVSS |
| VHH 09 | YYCK-------LTHPVYL---------VWGQGTQVTVSS |
| VHH 20 | YYCK-------LTHPVYL---------VWGQGTQVTVSS |
| VHH 32 | YYCKASIGSTRIRDTYYYR-------DYWGQGIQVTVSS |
| VHH 30 | YYCALDR----FYGSVLRGS-----PDYWGQGTQVTVSS |
| VHH 28 | YYCAAX-----RGGSYYSWDGSLTDFGSWGQGTXFTVSS |
| VHH 29 | YYCY---------ARRLN---------SWGQGTQVTISS |
| 3.F5 | YYCSGFVRTRDDPSRIRN---------YWGQGTQVTVST |
| 3.A9 | YYCHAFSR-----SRFEG---------YWGQGIQVTVSS |
| VHH 14 | YYCAAAPVFAPLTAQHIRLIS---MYEYWGQGTQVTVSS |
| VHH 12 | YVCAAHS-DYDCFSVEYG---------YWGQGTQVTVSS |

1. A heavy chain variable domain antibody (VHH) comprising at least a CDR1, CDR2 or CDR3 sequence as depicted in table 2, table 5.3, table 10, table 13 or table 14.

2. A VHH according to claim 1, comprising a sequence as depicted in table 2, table 5.3, table 10, table 13 or table 14 or a derivative thereof.

3. A VHH according to claim 1, comprising a sequence as depicted in table 2, table 5.3, table 10, table 13 or table 14, comprising a hallmark amino acid residue selected from the amino acids depicted for the corresponding position in table 3, preferably in the combination as depicted in table 5.2.

4. A VHH according to claim 1, comprising a sequence as depicted in table 2, table 5.3, table 10, table 13 or table 14, comprising an amino acid residue selected from the amino acids depicted for the corresponding position in table 6 for framework 1, table 7 for framework 2, table 8 for framework 3 and/or table 9 for framework 4.

5. A VHH according to claim 3, wherein said amino acid residue of table 3, table 6, table 7, table 8 or table 9 replaces the corresponding amino acid of table 2, table 5.3, table 10, table 13 or table 14.

6. A VHH according to claim 2, comprising an amino acid residue depicted for camelid VHHs in any of table 6-9.

7. A VHH according to claim 2, comprising between 1 and 5 amino acid substitutions compared to the sequence as depicted in table 2, table 5.3, table 10, table 13 or table 14.

8. A VHH according to claim 1, that is specific for PABPN1, beta-amyloid or emerin.

9. A VHH specific for PABPN1, beta-amyloid or emerin capable of blocking the binding of a VHH according to claim 1, to its target.

10. A VHH according to claim 1, comprising a signal sequence for directing the VHH to a specific location in a cell.

11. A VHH according to claim 10, wherein said signal sequence directs said VHH to the nucleus, the endoplasmic reticulum and/or the exterior of a cell.

12. A VHH according to claim 10, wherein said VHH is provided with said signal sequence.

13. A VHH according to claim 1, that is humanized or de-immunized.

14. A VHH according to claim 1, comprising the CDR1, CDR2 and CDR3 sequence of a VHH depicted in table 2, table 5.3, table 10, table 13 or table 14.

15. A nucleic acid encoding a VHH according to claim 1.

16. A vector, preferably an expression vector comprising a nucleic acid according to claim 15.

17. A recombinant and/or isolated cell provided with a nucleic acid according to claim 15.

18. A recombinant and/or isolated cell comprising a VHH according to claim 1.

19. A recombinant and/isolated cell according to claim 18, provided with a VHH.

20. A recombinant and/or isolated cell provided with a vector according to claim 16.

21. An isolated and/or recombinant gene delivery vehicle comprising a nucleic acid according to claim 15.

22. A method for producing a VHH comprising providing a cell with a nucleic acid according to claim 15 and culturing said cell to allow production of said VHH.

23. A method for identifying a region on a protein that is involved with the aggregation of said protein comprising contacting said protein with a VHH according to claim 1.

24. A method for selecting a compound from a collection of compounds said method comprising
providing a first and a second member of a specific binding pair,
contacting said first member of said specific binding pair with said compound, and
determining whether said compound inhibits binding of said binding pair,
wherein said first member comprises a protein that is associated with the formation of aggregates in a disease that is accompanied by the formation of said aggregates, or wherein said first member is a functional part, derivative and/or analogue of said protein, and wherein said second member comprises a VHH according to claim 1.

25. A method according to claim 24, further comprising determining whether said compound at least in part prevents the formation of said aggregates.

26. A method according to claim 24, further comprising determining whether said compound at least in part dissolves said aggregates.

27. A method for selecting an antigen specific VHH carrier from a display library comprising a plurality of VHH carriers said method comprising at least two successive rounds of antigen binding directed selection of VHH carriers and at least one round of function directed screening of VHH and/or VHH carriers, wherein in one round of selection VHH carriers are selected from said library through contacting VHH carriers with directionally immobilized antigen, wherein in another round of selection, antigen specific VHH carriers are selected by contacting VHH carriers with passively immobilized antigen and wherein said at least one round of function directed screening comprises testing two or more VHH or VHH carriers for the property to at least in part prevent aggregation of protein comprising said antigen and/or for the property to at least in part dissolve aggregates comprising protein comprising said antigen.

28. A method according to claim 27, wherein in one round of selection a subset of VHH carriers is selected from said library through contacting said library with directionally immobilized antigen and wherein in a subsequent round of selection said antigen specific VHH carrier is selected from said subset by contacting said subset or a part thereof with passively immobilized antigen.

29. A method according to claim 27, wherein effectiveness of selection of at least one round of selection is verified by contacting a sample of selected VHH carriers or VHH produced therefrom with a preparation of antigen and determining binding of said VHH.

30. A method according to claim 27, wherein said directionally immobilized antigen is immobilized on a solid surface by means of a binding body that is specific for an epitope on said antigen.

31. A method according to claim 27, wherein said antigen comprises a protein or a part comprising at least 10 consecutive amino acids thereof encoded by a primate gene.

32. A method according to claim 31, wherein said primate gene is a human gene.

33. A method according to claim 31, wherein a protein encoded by said gene is associated with a disease in humans.

34. A method according to claim 33, wherein said disease is associated with accumulation of aggregates comprising said protein or a mutant thereof.

35. A method according to claim 31, wherein said gene is a gene of table 1.

36. A method according to claim 31, wherein said gene encodes a product with an extension of a naturally occurring amino acid repeat.

37. A method according to claim 36, wherein said extension comprises an extension of an Alanine, a Glutamine or a Histidine repeat.

38. A method according to claim 35, wherein said gene is a gene is associated with a Poly GIn or a Poly Ala disease depicted in table 1.

39. A method according to claim 35, wherein said gene is amyloid β, Tau or α-synuclein.

40. A method according to claim 36, wherein said gene is PABPN1, ARX, ACTA1, HOXD 13, RUNX2, SOX3, HOXA, FOXL2 or IT15.

41. A method according to claim 34, wherein said disease is associated with aggregates comprising a mutant of said protein.

42. A method according to claim 41, wherein at least one round of selection comprising contacting VHH carriers with antigen of a protein encoded by a normal primate gene.

43. A method for selecting an antigen specific VHH carrier from a display library comprising a plurality of VHH carriers said method comprising selecting said antigen specific VHH carrier from said display library by means of at least two successive rounds of antigen binding directed selection of VHH carriers and at least one round of function directed screening of VHH and/or VHH carriers,
wherein said antigen is an antigen of a protein encoded by a primate gene;
wherein a mutated form of said gene in humans is associated with accumulation of aggregates in humans;
wherein said antigen is an antigen of a protein encoded by the normal primate gene; and
wherein said at least one round of function directed screening comprises testing two or more VHH or VHH carriers for the property to at least in part prevent aggregation of protein comprising said antigen and/or for the property to at least in part dissolve aggregates comprising protein comprising said antigen.

44. A method according to claim 43, wherein said gene is a human gene.

45. A method according to claim 43, wherein said antigen comprises said protein.

46. A method according to claim 43, wherein said antigen specific VHH carrier is selected by a method according to any one of claims 27-27-42.

47. A method according to claim 27, wherein at least one epitope on said antigen is masked prior to contacting VHH with said antigen in a selection round.

48. A method according to claim 47, wherein said epitope is masked through binding of a VHH specific for said antigen.

49. A method according to claim 47, wherein two or more epitopes an said antigen are masked.

50. A method according to claim 47, wherein at least one epitope not involved in aggregation of said protein is masked.

51. A method according to claim 47, wherein at least one of said masked epitopes is an immunodominant epitope.

52. A method according to claim 27, wherein at least one selection round comprises a proteinaceous complex comprising said antigen.

53. A method according to claim 52, wherein antigen in said complex comprises protein in a natural conformation.

54. A method according to claim 27, further comprising producing said antigen specific VHH.

55. A method according to claim 27, further comprising producing a selected antigen specific VHH.

56. A method according to claim 55, further comprising determining whether said VHH is capable of at least reducing the formation of aggregates comprising said protein.

57. A method according to claim 55, further comprising determining whether said VHH is capable of at least decreasing the size of formed aggregates comprising said protein.


SEQUENCE LISTING (128 sequences)

SEQ ID NO: 1 - DNA, 30 bp, Artificial
  Primer EMERINFBAM
cgcggatccatggacaactacgcagatctt

SEQ ID NO: 2 - DNA, 32 bp, Artificial
  Primer EMERINXHO
ccgccctcgaggtccagggagctcctggaggc

SEQ ID NO: 3 - DNA, 30 bp, Artificial
  Primer ACT1
cgcggatcctgcgacgaagacgagaccacc

SEQ ID NO: 4 - DNA, 31 bp, Artificial
  Primer ACT2
cgcaagcttggaagcatttgcggtggacgat

SEQ ID NO: 5 - DNA, 30 bp, Artificial
  Primer TP1
cgcggatccgacgccatcaagaagaagatg

SEQ ID NO: 6 - DNA, 31 bp, Artificial
  Primer TP2
cgcaagcttgcatggaagtcatatcgttgag

SEQ ID NO: 7 - DNA, 17 bp, Artificial
  Primer M13Rev
caggaaacagctatgac

SEQ ID NO: 8 - DNA, 21 bp, Artificial
  Primer MPE25
tttctgtatggggttttgcta

SEQ ID NO: 9 - protein, 9 aa, Artificial
  CDR start
CAASGRTFS

SEQ ID NO: 10 - protein, 5 aa, Artificial
  CDR2 start
KEREF

SEQ ID NO: 11 - protein, 6 aa, Artificial
  Extension
HHHHHH

SEQ ID NO: 12 - protein, 9 aa, Artificial
  Extension
HHAAMAAHH

SEQ ID NO: 13 - protein, 123 aa, Artificial
  Sequence 3.F5
EVQLVESGGGLVQAGGSLRLSCAASGRTFSGYGMGWFRQAPGKEREFVAAISWRGGNTYYADSVKGRFTISRDNAKNTVWLQMNSLKPEDTAVYYCSGFVRTRDDPSRIRNYWGQGTQVTVST

SEQ ID NO: 14 - protein, 118 aa, Artificial
  Sequence 3.A9
QVQLVESGGDLVQAGGSLRLSCAASGHTFDSYGMGWFRQRPGKGREFVAAITMIGGSTHYADSAKGRFTISRDNTKNTISLQMNSLKPEDTAVYYCHAFSRSRFEGYWGQGIQVTVSS

SEQ ID NO: 15 - protein, 113 aa, Artificial
  Sequence 3.E9
QVQLQESGGGLVQPGGSLRLSCVASGFTFSDNAMSWVRRAPGKGLEWVSAINRAGDSARYADSVKGRFTISRDNAKNTLYLQMNSLKPEDTAVYYCTNGGNYRGQGTQVTVSA

SEQ ID NO: 16 - protein, 115 aa, Artificial
  Sequence #08
EVQLVESGGGLVQPGGSLRLSCVASGRTSRISRMAWFRQVPGNERELVATMSSSGITSYAGSVKGRFTISRDNAKNTVDLQMNSLKPEDTAVYYCKYSSRWNIYWGQGTQVTVSS

SEQ ID NO: 17 - protein, 115 aa, Artificial
  Sequence #18
AVQLVESGGGLVQAGGSLRLSCAASGSIVSLATMGWYRQAPGNQRELVATMSSSGITSYAGSVKGRFTISRDNAKNTVDLQMNSLKPEDTAVYYCKYSSRWNIYWGQGTLVTVSS

SEQ ID NO: 18 - protein, 114 aa, Artificial
  Sequence #29
QVQLVESGGGLVQAGDSLRLSCAASGRTFSSYVMGWFRQAPGKEREFVAAVTGGGISTYYADSVKGRFTISRDNAKNTVYLQMNSLTPEDTAVYYCYARRLNSWGQGTQVTISS

SEQ ID NO: 19 - protein, 4 aa, Artificial
  KERE sequence
KERE

SEQ ID NO: 20 - protein, 4 aa, Artificial
  KQRE sequence
KQRE

SEQ ID NO: 21 - protein, 4 aa, Artificial
  GLEW sequence
GLEW

SEQ ID NO: 22 - protein, 5 aa, Artificial
  KEREL sequence
KEREL

SEQ ID NO: 23 - protein, 5 aa, Artificial
  KEREF sequence
KEREF

SEQ ID NO: 24 - protein, 5 aa, Artificial
  KQREL sequence
KQREL

SEQ ID NO: 25 - protein, 5 aa, Artificial
  KQREF sequence
KQREF

SEQ ID NO: 26 - protein, 5 aa, Artificial
  KEREG sequence
KEREG

SEQ ID NO: 27 - protein, 4 aa, Artificial
  TERE sequence
TERE

SEQ ID NO: 28 - protein, 5 aa, Artificial
  TEREL sequence
TEREL

SEQ ID NO: 29 - protein, 4 aa, Artificial
  KECE sequence
KECE

SEQ ID NO: 30 - protein, 5 aa, Artificial
  KECEL sequence
KECEL

SEQ ID NO: 31 - protein, 5 aa, Artificial
  KECER sequence
KECER

SEQ ID NO: 32 - protein, 4 aa, Artificial
  RERE sequence
RERE

SEQ ID NO: 33 - protein, 5 aa, Artificial
  REREG sequence
REREG

SEQ ID NO: 34 - protein, 4 aa, Artificial
  QERE sequence
QERE

SEQ ID NO: 35 - protein, 5 aa, Artificial
  QEREG sequence
QEREG

SEQ ID NO: 36 - protein, 4 aa, Artificial
  KGRE sequence
KGRE

SEQ ID NO: 37 - protein, 5 aa, Artificial
  KGREG sequence
KGREG

SEQ ID NO: 38 - protein, 4 aa, Artificial
  KDRE sequence
KDRE

SEQ ID NO: 39 - protein, 5 aa, Artificial
  KDREV sequence
KDREV

SEQ ID NO: 40 - protein, 5 aa, Artificial
  DECKL sequence
DECKL

SEQ ID NO: 41 - protein, 5 aa, Artificial
  NVCEL sequence
NVCEL

SEQ ID NO: 42 - protein, 4 aa, Artificial
  GVEW sequence
GVEW

SEQ ID NO: 43 - protein, 4 aa, Artificial
  EPEW sequence
EPEW

SEQ ID NO: 44 - protein, 4 aa, Artificial
  GLER sequence
GLER

SEQ ID NO: 45 - protein, 4 aa, Artificial
  DQEW sequence
DQEW

SEQ ID NO: 46 - protein, 4 aa, Artificial
  DLEW sequence
DLEW

SEQ ID NO: 47 - protein, 4 aa, Artificial
  GIEW sequence
GIEW

SEQ ID NO: 48 - protein, 4 aa, Artificial
  ELEW sequence
ELEW

SEQ ID NO: 49 - protein, 4 aa, Artificial
  GPEW sequence
GPEW

SEQ ID NO: 50 - protein, 4 aa, Artificial
  EWLP sequence
EWLP

SEQ ID NO: 51 - protein, 4 aa, Artificial
  GPER sequence
GPER

SEQ ID NO: 52 - protein, 124 aa, Artificial
  Amyloid 42 VHH sequence 1B
QVQLQESGGGLVQAGGSLRLSCAASGRTFSSYVMGWFRQAPGKEREFVAAIGWSGGSTAYADSVKGRFTISRDDARNTVYLQMNSLKPEDTAVYYCASAPTRWVPRDSRFYDRWGQGTQVTVSS

SEQ ID NO: 53 - protein, 126 aa, Artificial
  Amyloid 42 VHH sequence 1D
QVQLQDSGGGLVQAGGSLRLSCAASGRTFSNYAMGWFRQAPEKEREFVAAINGSGGSTYYADSVKGRFTISRDDAKNTVYLQMNSLKPEDTAVYYCASKLYGSGTPRDTGDKYSNWGQGTQVTVSS

SEQ ID NO: 54 - protein, 124 aa, Artificial
  Amyloid 42 VHH sequence 2A
QVQLQDSGGGLVQAGGSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTQVTVSS

SEQ ID NO: 55 - protein, 115 aa, Artificial
  Amyloid 42 VHH sequence 2B
QVKLEESGGGLVQPGGSLRLSCAASGRTRTIRAMAWFRQALGKQREFIARITVGGTNYADSVKDRFTISRDDAKNTVYLQMNSLKPEDTAVYYCYSKTWGDRNYWGQGTQVTVPS

SEQ ID NO: 56 - protein, 124 aa, Artificial
  Amyloid 42 VHH sequence 2F
QVQLQESGGGLVQAGGSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTQVTVSS

SEQ ID NO: 57 - protein, 124 aa, Artificial
  Amyloid 42 VHH sequence 3A
AVQLVESGGGLVRDGGSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTQVTVSS

SEQ ID NO: 58 - protein, 123 aa, Artificial
  Amyloid 42 VHH sequence 3H
QVQLQESGGGLVQAGGSLRLSCTASGRTFSSWAMAWFRQAPGKEREFLAASWSGGNTAYANSVKGRFTISRDNAKNTVYLQMNSLKPEDTAVYYCASTGTRWVPRDSRFYDRWGQGTRVTVSS

SEQ ID NO: 59 - protein, 119 aa, Artificial
  Amyloid 42 VHH sequence 4C
QVQLQESGGGLVQAGGSLRLSCAASGRTFSRYAMGWFRQAPGKEREFVARIRWSGGSTYYADSVKGRFTISRDNAKNTVYLQMNSLKPEDTAVYYCAAGGTSGLSFDYWGQGTQVTVSS

SEQ ID NO: 60 - protein, 124 aa, Artificial
  Amyloid 42 VHH sequence 8F
QVQLQDSGGGLVQAGGSLRLSCAASGRTFSSLVMGWFRQASGKEREFVAAIGWSGGSTAYADSVKGRFTISRDNARNTVYLQMNSLKPEDTAVYYCASAPTRWVPRDSRFYDRWGQGTQVTVSS

SEQ ID NO: 61 - protein, 124 aa, Artificial
  Amyloid 42 VHH sequence 11D
AVQLVESGGGLVQAGGSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTQVTVSS

SEQ ID NO: 62 - protein, 118 aa, Artificial
  Amino acid sequence EME7E of the isolated VHH for emerin
QVQLVESGGGLVQAGGSLRLSCAASGRTFSSYTMGWFRQAPGKEREFVAGINWSGVRTYYGDSVKGRFTISRDNAKNTMYLQMNSLKAEDTAVYYCNVRGRYSNTDYWGQGIQVTVSS

SEQ ID NO: 63 - protein, 125 aa, Artificial
  Amino acid sequence EME1C of the isolated VHH for emerin
DVQLVESGGGLVQPGGSLRLSCAASGSIFRLNDMGWYRQPPGKQREMVATITKGGTTNYADSVKARFTISRDNAKNTVYLQMNSLKPEDTAVYYCNADISTYSAFGLFTPPKNYWGQGTQVTVSS

SEQ ID NO: 64 - protein, 125 aa, Artificial
  Amino acid sequence VHH01 of the isolated VHH for emerin
QVQLVESGGGLVQPGGSLRLSCAASGSIFRLNDMGWYRQPPGKQREMVATITKGGTTNYADSVKARFTISRDNAKNTVYLQMNSLKPEDTAVYYCNADISTYSAFGLFTPPKNYWGQGTQVTVSS

SEQ ID NO: 65 - protein, 125 aa, Artificial
  Amino acid sequence VHH03 / VHH23 of the isolated VHH for emerin
QVQLQESGGGLVQPGGSLRLSCAASGSIFRLNDMGWYRQPPGKQREMVATITKGGTTNYADSVKARFTISRDNAKNTVYLQMNSLKPEDTAVYYCNADISTYSAFGLFTPPKNYWGQGTQVTVSS

SEQ ID NO: 66 - protein, 125 aa, Artificial
  Amino acid sequence EME3H of the isolated VHH for emerin
QVQLQESGGSLVQPGGSLRLSCAASGSIFRLNDMRWYRQAPGKQREVVATITSGGTTNYADSVKARFTISRDNAKNTVYLQMNSLKPEDTAVYYCNADISLYSAFGLFSPPKNYWGQGTQVTVSS

SEQ ID NO: 67 - protein, 125 aa, Artificial
  Amino acid sequence VHH09 of the isolated VHH for emerin
QVQLQESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGTQVTVSS

SEQ ID NO: 68 - protein, 125 aa, Artificial
  Amino acid sequence VHH12 of the isolated VHH for emerin
QVQLQESGGSLVQPGGSLRLSCAASGSIFRLNDMRWYRQAPGKQREMVATITSGGTTNYADSVKARFTISRDNAKNTVYLQMNSLKPEDTAVYYCNADISLYSAFGLFSPPKNYWGQGTQVTVSS

SEQ ID NO: 69 - protein, 125 aa, Artificial
  Amino acid sequence VHH05 of the isolated VHH for emerin
QVQLVESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGIQVTVSS

SEQ ID NO: 70 - protein, 125 aa, Artificial
  Amino acid sequence VHH11 of the isolated VHH for emerin
AVQLVESGGSLVQPGGGLRLSCAASGSIFRLNDMRWYRQAPGKQREMVATITSGGTTNYADPVKARFTISRDNAKSTVYLQMNSLKPEDTAVYYCNADISLYSAFGLFSPPKNYWGQGIQVTVSS

SEQ ID NO: 71 - protein, 125 aa, Artificial
  Amino acid sequence EME8A of the isolated VHH for emerin
DVQLVESGGSLVQPGGGLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGTQVTVPS

SEQ ID NO: 72 - protein, 117 aa, Artificial
  Amino acid sequence VHH02 of the isolated VHH for emerin
QVQLVESGGGLVQPGGSLRLSCAASGSGFSINVMGWYRQYPGKQREVVAALSAGGSFYYTDSVKGRFTITRDNAKNTMYLQMNSLKPEDTALYYCAAGLLGQRPDYWGQGTQVTISS

SEQ ID NO: 73 - protein, 117 aa, Artificial
  Amino acid sequence VHH15 of the isolated VHH for emerin
QVQLVESGGGLVQAGGSLRLSCAASESIFSFKVMGWYRQYPGKQREVVAALSAGGSFYYTDSVKGRFTITRDNAKNTMYLQMNSLKPEDTALYYCAAGLLGQRPDYWGQGTRVTVSS

SEQ ID NO: 74 - protein, 120 aa, Artificial
  Amino acid sequence VHH10 of the isolated VHH for emerin
QVQLVESGGGLAQPGGSLSLSCAASGDIFSIYAMGWYRQAPGKKRELVADITNYGSTNYADSVKGRFTISRDNAKTTIYLQMNSLKPEDTAVYYCNADSYSKLRGWVVYWGQGTQVTVST

SEQ ID NO: 75 - protein, 118 aa, Artificial
  Amino acid sequence EME4B of the isolated VHH for emerin
EVQLVESGGGLVQTGDSLRLSCAVSGRTLSSFALGWFRQSPGKEREFVAALTWDGDRTSYSESVKGRFTISRDHAKNTVYLQMNSLKPEDTAVYYCAAGDLGRVPHYWGQGTRVTVSS

SEQ ID NO: 76 - protein, 122 aa, Artificial
  Amino acid sequence VHH13 of the isolated VHH for emerin
EVQLVESGGGLAQAGGSLRLSCAASGRTSEIYTMGWFRQAPGKEREFVAGIRWRSDRAAYEDSVKGRFTISRDNANNRVYLQMNSLKPEDTAVYYCAASSIAYRNDMSVSIWGQGTRVTVSS

SEQ ID NO: 77 - protein, 116 aa, Artificial
  Amino acid sequence EME7F of the isolated VHH for emerin
AVQLVESGGGLVQAGGSLRLSCAASGRTLRSYAVGWFRQAPGKEREFVAGINWNGDSTYYSDSVKGRFTISRDNAKNTVYLQMNSLKPDDTAVYYCNAGYIGNYYWGQGTQVTVSS

SEQ ID NO: 78 - protein, 116 aa, Artificial
  Amino acid sequence VHH14 of the isolated VHH for emerin
AVQLVESGGGLVQAGGSLRLSCAASGRTFSSYAVGWFRQAPGKDREFVAGINWTGANTYHADSVAGRVTISRDNAKNVVYLQMNSLKPEDTAVYYCNAGTQGGWYWGQGIQVTVSS

SEQ ID NO: 79 - protein, 122 aa, Artificial
  Amino acid sequence EME2G of the isolated VHH for emerin
DVQLVESGGGLVQAGGSLRLSCATSGRSLSSYAMAWFRQAPGKEREFVAGINFSGRRTYYTDSVKGRFNITRDNAKNTVYLQMNSLKPEDTAVYYCNVGGRKVLAGTFFNDWGQGTQVTVST

SEQ ID NO: 80 - protein, 120 aa, Artificial
  Amino acid sequence EME8D of the isolated VHH for emerin
EVQLVESGGGLVQAGGSLTLSCAASGRTLSSYAMGWFRQAPGKEREFVAGINWSGGSTYYADSVKGRFAISRDNAKNTVYLRMNSLKPEDTAVYYCNAGRFERLRLISYWGQGTQVTVST

SEQ ID NO: 81 - protein, 120 aa, Artificial
  Amino acid sequence VHH04 of the isolated VHH for emerin
QVQLVESGGGLVQAGGSLRLSCAASGRTFSDYTMGWFRQAPGKEREFVAGINGSGGRTYYADSVKDRFTISRDNAKNTVYLQMNNLKLEDTAVYYCNAGRFERLRLISYWGQGTQVTVPS

SEQ ID NO: 82 - protein, 116 aa, Artificial
  Amino acid sequence VHH07 / VHH08 of the isolated VHH for emerin
QVQLVESGGGLVQAGDSLRLSCAASGRTFSSYAMGWFRQAPGKEREFVAGISWSGGNTYYSDSVKGRFTISRDNTKNTVYLQMNSLKPEDTAVYSCNAGTYYKNYWGQGTQVTVSS

SEQ ID NO: 83 - protein, 125 aa, Artificial
  Amino acid sequence VHH16 of the isolated VHH for emerin
QVQLVESGGGLVQPGGSLRLSCAASGSIFRLNDMGWYRQPPGKQREMVATITKGGTTNYADSVKARFTISRDNAKNTVYLQMNSLKPEDTAVYYCNADISTYSAFGLFTPPKNYWGQGIQVTVSS

SEQ ID NO: 84 - protein, 125 aa, Artificial
  Amino acid sequence 3.6B of the isolated VHH for emerin
QVQLVESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGTQVTVST

SEQ ID NO: 85 - protein, 125 aa, Artificial
  Amino acid sequence 3.8B of the isolated VHH for emerin
DVQLVESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGIQVTVSS

SEQ ID NO: 86 - protein, 125 aa, Artificial
  Amino acid sequence VHH24 of the isolated VHH for emerin
QVQLVESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGTQVTVSS

SEQ ID NO: 87 - protein, 125 aa, Artificial
  Amino acid sequence VHH21 of the isolated VHH for emerin
QVQLQESGGSLVQPGGSLRLSCAASGSIFRLNDMRWYRQAPGKQREMVATITSGGTTNYADSVKARFTISRDNAKSTVYLQMNSLKPEDTAVYYCNADISLYSAFGLFSPPKNYWGQGIQVTVSS

SEQ ID NO: 88 - protein, 128 aa, Artificial
  Amino acid sequence 3.8E of the isolated VHH for emerin
QVQLQESGGELVQAGGSLRLSCAASGRTLSSYTIGWFRQAPGNVREFVAHHFASGGVTDYADFVKGRFTISRDNAKNTVYLEMNSLKPEDTAVYYCAASTFTIPGYRNLKAAYEYDYWGQGTQVTVSS

SEQ ID NO: 89 - protein, 122 aa, Artificial
  Amino acid sequence of VHH domains which can translocate via Blood Brain Barrier to the brain
EVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 90 - protein, 125 aa, Artificial
  Amino acid sequence of VHH domains which can translocate via Blood Brain Barrier to the brain
EVQLQASGGGLVQAGGSLRLSCSASVRTFSIYTMGWFRQAPGKEREFVAGINRSGDVTKYADFVKGRFSISRDHAKNMVYLQMNSLKPEDTALYYCAATWAYDTVGALTSGYNFWGQGTQVTVSS

SEQ ID NO: 91 - DNA, 372 bp, Artificial
  1B 1-40, anti-B-amyloid
caagttcaactgcaggaatctggtggtggtcttgtacaaccaggtggttctttgaggctaagctgtgctgcttctgaattcactttggattactactctattgcttggttcagacaagctcccgggaaggaaagagaaggtgtttcttgtatttcttcttacgatggttctacttcttacgctgactctgtcaagggtagattcactatttctagagataacgctaagaacactgtatacttgcaaatgaactctttgaagcctgaggatactgctatttactactgtgcggccggtattagagattgggctactttgagagaatacgaatacgatgattggggtcaaggtacccaggtcaccgtttcttct

SEQ ID NO: 92 - protein, 124 aa, Artificial
  Synthetic Construct
QVQLQESGGGLVQPGGSLRLSCAASEFTLDYYSIAWFRQAPGKEREGVSCISSYDGSTSYADSVKGRFTISRDNAKNTVYLQMNSLKPEDTAIYYCAAGIRDWATLREYEYDDWGQGTQVTVSS

SEQ ID NO: 93 - DNA, 372 bp, Artificial
  3A 1-42, anti-B-amyloid
gctgttcaactgcaggaatctggtggtggtcttgtacaagatggtgattctttgaggctaagctgtgctgcttctggtagaactttctcttcttacgttatgggttggttcagacaagctcccgggaaggaaagagaattcgttgctgctattggttggtctggtggttctactgcttacgctgactctgtcaagggtagattcactatttctagagataacgctagaaacactgtatacttgcaaatgaactctttgaagcctgaggatactgctgtttactactgtgcttctgctccaactagatgggttccaagagattctagattctacgatagatggggtcaaggtacccaggtcaccgtttcttct

SEQ ID NO: 94 - protein, 124 aa, Artificial
  Synthetic Construct
AVQLQESGGGLVQDGDSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTQVTVSS

SEQ ID NO: 95 - DNA, 360 bp, Artificial
  FC5, anti-FC5-receptor
gaagttcaactgcaggcttctggtggtggtcttgtacaagctggtggttctttgaggctaagctgtgctgcttctggtttcaagattactcactacaccatgggttggttcagacaagcccccgggaaggaaagagaattcgtttccagaattacttggggtggtgataacactttctactctgactctgtcaagggtcgattcactatttccagagataacgctaagaacactgtatacttgcaaatgaactctttgaagcctgaggatactgctgattactactgcgcggccggttctacttctactgctactccattgagagttgattactggggtaagggtacccaggtctcttct

SEQ ID NO: 96 - protein, 120 aa, Artificial
  Synthetic Construct
EVQLQASGGGLVQAGGSLRLSCAASGFKITHYTMGWFRQAPGKEREFVSRITWGGDNTFYSDSVKGRFTISRDNAKNTVYLQMNSLKPEDTADYYCAAGSTSTATPLRVDYWGKGTQVSS

SEQ ID NO: 97 - protein, 124 aa, Artificial
  VHH07
QVQLQESGGGLVQPGGSLRLSCAASRFTLDNYAVGWFRQAAGKEREAVSCSSSGDGRTYYADSVKGRFTISRDNAKDTVYLQMNSLKPEDTAVYYCATGFFVRSCSSPDSYDYWGQGTQVIVSS

SEQ ID NO: 98 - protein, 124 aa, Artificial; identical to SEQ ID NO: 97
  VHH24

SEQ ID NO: 99 - protein, 124 aa, Artificial
  VHH15
QVQLQESGGGAVQPGGSLRLSCAASRFTLDNYAVGWFRQAPGKEREAVSCSSSSDGRTYYADSVKGRFTISRDNAKDTVYLQMNSLKPEDTAVYYCATGFFVRSCRSPDSYDYWGQGTQVIVSS

SEQ ID NO: 100 - protein, 124 aa, Artificial; identical to SEQ ID NO: 99
  VHH33

SEQ ID NO: 101 - protein, 124 aa, Artificial; identical to SEQ ID NO: 99
  VHH31

SEQ ID NO: 102 - protein, 124 aa, Artificial
  VHH01
QVQLQESGGGLVQPGGSLRLSCAASRFTLDNYAVGWFRQAPGKEREAVSCSSSSDGPTYYADSVKGRFTISRDNAKDTVYLQMNSLKPEDTAVYYCATGFFVRSCSSPDSYDYWGQGTQVIVSS

SEQ ID NO: 103 - protein, 124 aa, Artificial
  VHH04
QVQLQESGGGLVQPGGSLRLSCAASRFTLDYYAVGWFRQAPGKEREAVSCSSSSDGRTYYADSVKGRFTISRDNAKDTVYLQMNSLKPEDTAVYYCATGFFVRSCSSPDSYDYWGQGTQVIVSS

SEQ ID NO: 104 - protein, 124 aa, Artificial
  VHH19
QVQLQESGGGLVQPGGSLRLSCAASRFTLDYYAVGWFRQAPGKEREAVSCSSSSDGHTYYADSVKGRFTISRDNAKDTVYLQMNSLKPEDTAVYYCATGFFVRSCSSPDSYDYWGQGTQVIVSS

SEQ ID NO: 105 - protein, 124 aa, Artificial
  VHH21
AVQLVESGGGLVQAGGSLRLSCAASGFTFDYYAVGWFRQAPGKEREAVSCSSSSDGRTYYADSVKGRFTISRDNAKDTVYLQMNSLKPEDTAVYYCATGFFVRSCSSPDSYDYWGQGTRVTVSS

SEQ ID NO: 106 - protein, 125 aa, Artificial
  VHH05
AVQLVESGGGLVQAGGSLRLSCAASGFTFDDYAIGWFRQAPGKEREGISCISKSDGNTNYADSVKGRFTISSDNAKNTVFLQMNSLKPEDTAVYYCAVGVLGSCPIMVYLLYDDWGQGTRVTVSS

SEQ ID NO: 107 - protein, 117 aa, Artificial
  VHH23
AVQLVESGGDLVQPGGSLRLSCTASGFTFNTYGMSWVRQAPGKGLVWVSSISSGGVPMYADSVKGRFTISRDNTKNTLYLQMNSLKPEDTAVYYCVKGYYTGSFLPPGQGTRVTVSS

SEQ ID NO: 108 - protein, 117 aa, Artificial
  VHH34
QVQLQESGGGLVQPGGSLRLSCAASGFTFSTYGMSWVRQAPGKGLRWVSSISSGGVPMYASSVKGRFTISRDNAKNTLYLQMNSLKPEDTAVYYCVKGYYTGDFLPPGQGTQVAVSS

SEQ ID NO: 109 - protein, 117 aa, Artificial
  VHH26
QVQLQESGGGLVQPGGSLRLSCAASGFTFSTYGMSWVRQAPGKGVEWVSSINSGGVVPMYAASVKGRFTISRDNANNTLTLQMNSLKPEDTAVYYCVKGYYTGNPPLGQGTQVTVSS

SEQ ID NO: 110 - protein, 113 aa, Artificial; identical to SEQ ID NO: 15
  3.E9

SEQ ID NO: 111 - protein, 115 aa, Artificial; identical to SEQ ID NO: 16
  VHH08

SEQ ID NO: 112 - protein, 115 aa, Artificial; identical to SEQ ID NO: 17
  VHH18

SEQ ID NO: 113 - protein, 115 aa, Artificial
  VHH09
QVQLQDSGGGLVQPGGSLRLSCAASGSIFSFSTMGWYRQAPGKQRELVAAITRTGTTNYADSVKGRFTISRDNTKNTVYLQMNSLKPEDTSAYYCKLTHPVYLVWGQGTQVTVSS

SEQ ID NO: 114 - protein, 115 aa, Artificial
  VHH20
QVQLQESGGGLVQPGGSLRLSCAASGSIFSFSTMGWYRQAPGKQRELVAAITRTGTTNYADSVKGRFTISRDNTKNTVYLQMNSLKPEDTSAYYCKLTHPVYLVWGQGTQVTVSS

SEQ ID NO: 115 - protein, 124 aa, Artificial
  VHH32
EVQLVESGGGLVQPGGSLRLSCAASGSIFRINTMAWYRQAPGKQRELVATITNGGNTNYADSVKGRFTISRDNAKNTVYLQMNSLKPEDTAVYYCKAEIGSTRIRDTYYYRDYWGQGIQVTVSS

SEQ ID NO: 116 - protein, 122 aa, Artificial
  VHH30
EVQLVESGGGLVQPGGSLRLSCAGSLDIFSVYVMGWYRQVPGGQRDLVATITKEGMPDYADSVKGRFTISRDNAENTVYLQMNSLKPEDTGVYYCALDRFYGSVLRGSPDYWGQGTQVTVSS

SEQ ID NO: 117 - protein, 127 aa, Artificial
  VHH28
QVQLQESGGGLVQAGGSLRLSCAASGLAFSRTAMAWFRQAPGKEREFVAAIAWSAGNTLYEESVKGRFTISRDNAKNTVYLQMNNLKPEDTAVYYCAAXRGGSYYSWDGSLTDFGSWGQGTXFTVSS

SEQ ID NO: 118 - protein, 114 aa, Artificial; identical to SEQ ID NO: 18
  VHH29

SEQ ID NO: 119 - protein, 123 aa, Artificial; identical to SEQ ID NO: 13
  3.F5

SEQ ID NO: 120 - protein, 118 aa, Artificial; identical to SEQ ID NO: 14
  3.A9

SEQ ID NO: 121 - protein, 128 aa, Artificial
  VHH14
QVQLQESGGGLVQAGGSLRLSCTASGHTLSSYAMGWFRQAPGKEREFVAAISWSDITYYEDSVKGRFTISRDNAKNTVYLQMNSLNPEDTAVYYCAAAPVFAPLTAQHIRLISMYEYWGQGTQVTVSS

SEQ ID NO: 122 - protein, 122 aa, Artificial
  VHH12
QVQLQESGGGLVQAGGSLRLSCTVSGFTVDAYAMGWFRQTPGKGHEVVACISSPDGITTYADSVKGRFTISRDSVQNTVSLQMDSLKPEDTAVYVCAAHSDYDCFSVEYGYWGQGTQVTVSS

SEQ ID NO: 123 - protein, 18 aa, Artificial
  PABPNI
VREMEEEAEKLKELQNEV

SEQ ID NO: 124 - protein, 143 aa, Artificial
  8B
MAAVQLVESGGGLVQAGGSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTRVTVSSYTDIEMNRLGKHHHHHH

SEQ ID NO: 125 - protein, 143 aa, Artificial
  3A
MAAVQLVESGGGLVRDGDSLRLSCAASGRTFSSYVMGWFRQAPGKEREFVAAIGWSGGSTAYADSVKGRFTISRDNARNTVYLQMNSLKPEDTAVYYCASAPTRWVPRDSRFYDRWGQGTRVTVSSYTDIEMNRLGKHHHHHH

SEQ ID NO: 126 - protein, 143 aa, Artificial
  1B
MAQVQLQESGGGLVQPGGSLRLSCAASEFTLDYYSIAWFRQAPGKEREGVSCISSYDGSTSYADSVKGRFTISRDNAKNTVYLQMNSLKPEDTAIYYCAAGIRDWATLREYEYDDWGQGTQVTVSSYTDIEMNRLGKHHHHHH

SEQ ID NO: 127 - protein, 141 aa, Artificial
  11G
MAQVQLQESGGGLVQPGGSLRLSCAASGSIFSINTMAWYRQAPGKERDLVAAIISSGSTNYADSVKGRFTISRDNTKNTVYLQMNSLKLEDTAVYYCNAAIRRSVIDDAWGAYWGQGTQVTVSSYTDIEMNRLGKHHHHHH

SEQ ID NO: 128 - protein, 139 aa, Artificial
  4D
MAQVQLQESGGGLVQAGGSLRLSCAASGRTFSSYAMGWFRQAPGKEREFVATIRWNGDYADSVRDRFTISRDDAKNTVFLQMNSLKPEDTAIYYCAARLGPRTSQAALYRYWGQGTQVTVSSYTDIEMNRLGKHHHHHH